SEQUENCE LISTING (16 sequences)

SEQ ID NO: 1 - DNA, 3888 bp, Clostridium botulinum
atgccattcgtcaacaagcaattcaactacaaagacccagtcaacggcgtcgacatcgcatacatcaagattccgaacgccggtcaaatgcagccggttaaggcttttaagatccacaacaagatttgggttatcccggagcgtgacaccttcacgaacccggaagaaggcgatctgaacccgccaccggaagcgaagcaagtccctgtcagctactacgattcgacgtacctgagcacggataacgaaaaagataactacctgaaaggtgtgaccaagctgttcgaacgtatctacagcacggatctgggtcgcatgctgctgactagcattgttcgcggtatcccgttctggggtggtagcacgattgacaccgaactgaaggttatcgacactaactgcattaacgttattcaaccggatggtagctatcgtagcgaagagctgaatctggtcatcattggcccgagcgcagacattatccaattcgagtgcaagagctttggtcacgaggttctgaatctgacccgcaatggctatggtagcacccagtacattcgtttttcgccggattttaccttcggctttgaagagagcctggaggttgataccaatccgttgctgggtgcgggcaaattcgctaccgatccggctgtcacgctggcccatgaactgatccacgcaggccaccgcctgtacggcattgccatcaacccaaaccgtgtgttcaaggttaatacgaatgcatactacgagatgagcggcctggaagtcagcttcgaagaactgcgcaccttcggtggccatgacgctaaattcattgacagcttgcaagagaatgagttccgtctgtactactataacaaattcaaagacattgcaagcacgttgaacaaggccaaaagcatcgttggtactaccgcgtcgttgcagtatatgaagaatgtgtttaaagagaagtacctgctgtccgaggatacctccggcaagtttagcgttgataagctgaagtttgacaaactgtacaagatgctgaccgagatttacaccgaggacaactttgtgaaattcttcaaagtgttgaatcgtaaaacctatctgaattttgacaaagcggttttcaagattaacatcgtgccgaaggtgaactacaccatctatgacggttttaacctgcgtaacaccaacctggcggcgaactttaacggtcagaatacggaaatcaacaacatgaatttcacgaagttgaagaacttcacgggtctgttcgagttctataagctgctgtgcgtgcgcggtatcatcaccagcaaaaccaaaagcctggacaaaggctacaacaaggcgctgaatgacctgtgcattaaggtaaacaattgggatctgttcttttcgccatccgaagataattttaccaacgacctgaacaagggtgaagaaatcaccagcgatacgaatattgaagcagcggaagagaatatcagcctggatctgatccagcagtactatctgacctttaacttcgacaatgaaccggagaacattagcattgagaatctgagcagcgacattatcggtcagctggaactgatgccgaatatcgaacgtttcccgaacggcaaaaagtacgagctggacaagtacactatgttccattacctgcgtgcacaggagtttgaacacggtaaaagccgtatcgcgctgaccaacagcgttaacgaggccctgctgaacccgagccgtgtctataccttcttcagcagcgactatgttaagaaagtgaacaaagccactgaggccgcgatgttcctgggctgggtggaacagctggtatatgacttcacggacgagacgagcgaagtgagcactaccgacaaaattgctgatattaccatcattatcccgtatattggtccggcactgaacattggcaacatgctgtacaaagacgattttgtgggtgccctgatcttctccggtgccgtgattctgctggagttcattccggagattgcgatcccggtgttgggtaccttcgcgctggtgtcctacatcgcgaataaggttctgacggttcagaccatcgataacgcgctgtcgaaacgtaatgaaaaatgggacgaggtttacaaatacattgttacgaattggctggcgaaagtcaatacccagatcgacctgatccgtaagaaaatgaaagaggcgctggagaatcaggcggaggccaccaaagcaattatcaactaccaatacaaccagtacacggaagaagagaagaataacattaacttcaatatcgatgatttgagcagcaagctgaatgaatctatcaacaaagcgatgatcaatatcaacaagtttttgaatcagtgtagcgtttcgtacctgatgaatagcatgattccgtatggcgtcaaacgtctggaggacttcgacgccagcctgaaagatgcgttgctgaaatacatttacgacaatcgtggtacgctgattggccaagttgaccgcttgaaagacaaagttaacaataccctgagcaccgacatcccatttcaactgagcaagtatgttgataatcaacgtctgttgagcactttcaccgagtatatcaaaaacatcatcaatactagcattctgaacctgcgttacgagagcaatcatctgattgatctgagccgttatgcaagcaagatcaacatcggtagcaaggtcaattttgacccgatcgataagaaccagatccagctgtttaatctggaatcgagcaaaattgaggttatcctgaaaaacgccattgtctacaactccatgtacgagaatttctccaccagcttctggattcgcatcccgaaatacttcaacagcattagcctgaacaacgagtatactatcatcaactgtatggagaacaacagcggttggaaggtgtctctgaactatggtgagatcatttggaccttgcaggacacccaagagatcaagcagcgcgtcgtgttcaagtactctcaaatgatcaacatttccgattacattaatcgttggatcttcgtgaccattacgaataaccgtctgaataacagcaagatttacatcaatggtcgcttgatcgatcagaaaccgattagcaacctgggtaatatccacgcaagcaacaacattatgttcaaattggacggttgccgcgatacccatcgttatatctggatcaagtatttcaacctgtttgataaagaactgaatgagaaggagatcaaagatttgtatgacaaccaatctaacagcggcattttgaaggacttctggggcgattatctgcaatacgataagccgtactatatgctgaacctgtatgatccgaacaaatatgtggatgtcaataatgtgggtattcgtggttacatgtatttgaagggtccgcgtggcagcgttatgacgaccaacatttacctgaactctagcctgtaccgtggtacgaaattcatcattaagaaatatgccagcggcaacaaagataacattgtgcgtaataacgatcgtgtctacatcaacgtggtcgtgaagaataaagagtaccgtctggcgaccaacgcttcgcaggcgggtgttgagaaaattctgagcgcgttggagatccctgatgtcggtaatctgagccaagtcgtggttatgaagagcaagaacgaccagggtatcactaacaagtgcaagatgaacctgcaagacaacaatggtaacgacatcggctttattggtttccaccagttcaacaatattgctaaactggtagcgagcaattggtacaatcgtcagattgagcgcagcagccgtactttgggctgtagctgggagtttatcccggtcgatgatggttggggcgaacgtccgctg

SEQ ID NO: 2 - protein, 1296 aa, Clostridium botulinum
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIINTSILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDQGITNKCKMNLQDNNGNDIGFIGFHQFNNIAKLVASNWYNRQIERSSRTLGCSWEFIPVDDGWGERPL

SEQ ID NO: 3 - DNA, 3888 bp, artificial
  Modified BoNT/A "Cat-A"
atgccattcgtcaacaagcaattcaactacaaagacccagtcaacggcgtcgacatcgcatacatcaagattccgaacgccggtcaaatgcagccggttaaggcttttaagatccacaacaagatttgggttatcccggagcgtgacaccttcacgaacccggaagaaggcgatctgaacccgccaccggaagcgaagcaagtccctgtcagctactacgattcgacgtacctgagcacggataacgaaaaagataactacctgaaaggtgtgaccaagctgttcgaacgtatctacagcacggatctgggtcgcatgctgctgactagcattgttcgcggtatcccgttctggggtggtagcacgattgacaccgaactgaaggttatcgacactaactgcattaacgttattcaaccggatggtagctatcgtagcgaagagctgaatctggtcatcattggcccgagcgcagacattatccaattcgagtgcaagagctttggtcacgaggttctgaatctgacccgcaatggctatggtagcacccagtacattcgtttttcgccggattttaccttcggctttgaagagagcctggaggttgataccaatccgttgctgggtgcgggcaaattcgctaccgatccggctgtcacgctggcccatgaactgatccacgcaggccaccgcctgtacggcattgccatcaacccaaaccgtgtgttcaaggttaatacgaatgcatactacgagatgagcggcctggaagtcagcttcgaagaactgcgcaccttcggtggccatgacgctaaattcattgacagcttgcaagagaatgagttccgtctgtactactataacaaattcaaagacattgcaagcacgttgaacaaggccaaaagcatcgttggtactaccgcgtcgttgcagtatatgaagaatgtgtttaaagagaagtacctgctgtccgaggatacctccggcaagtttagcgttgataagctgaagtttgacaaactgtacaagatgctgaccgagatttacaccgaggacaactttgtgaaattcttcaaagtgttgaatcgtaaaacctatctgaattttgacaaagcggttttcaagattaacatcgtgccgaaggtgaactacaccatctatgacggttttaacctgcgtaacaccaacctggcggcgaactttaacggtcagaatacggaaatcaacaacatgaatttcacgaagttgaagaacttcacgggtctgttcgagttctataagctgctgtgcgtgcgcggtatcatcaccagcaaaaccaaaagcctggacaaaggctacaacaaggcgctgaatgacctgtgcattaaggtaaacaattgggatctgttcttttcgccatccgaagataattttaccaacgacctgaacaagggtgaagaaatcaccagcgatacgaatattgaagcagcggaagagaatatcagcctggatctgatccagcagtactatctgacctttaacttcgacaatgaaccggagaacattagcattgagaatctgagcagcgacattatcggtcagctggaactgatgccgaatatcgaacgtttcccgaacggcaaaaagtacgagctggacaagtacactatgttccattacctgcgtgcacaggagtttgaacacggtaaaagccgtatcgcgctgaccaacagcgttaacgaggccctgctgaacccgagccgtgtctataccttcttcagcagcgactatgttaagaaagtgaacaaagccactgaggccgcgatgttcctgggctgggtggaacagctggtatatgacttcacggacgagacgagcgaagtgagcactaccgacaaaattgctgatattaccatcattatcccgtatattggtccggcactgaacattggcaacatgctgtacaaagacgattttgtgggtgccctgatcttctccggtgccgtgattctgctggagttcattccggagattgcgatcccggtgttgggtaccttcgcgctggtgtcctacatcgcgaataaggttctgacggttcagaccatcgataacgcgctgtcgaaacgtaatgaaaaatgggacgaggtttacaaatacattgttacgaattggctggcgaaagtcaatacccagatcgacctgatccgtaagaaaatgaaagaggcgctggagaatcaggcggaggccaccaaagcaattatcaactaccaatacaaccagtacacggaagaagagaagaataacattaacttcaatatcgatgatttgagcagcaagctgaatgaatctatcaacaaagcgatgatcaatatcaacaagtttttgaatcagtgtagcgtttcgtacctgatgaatagcatgattccgtatggcgtcaaacgtctggaggacttcgacgccagcctgaaagatgcgttgctgaaatacatttacgacaatcgtggtacgctgattggccaagttgaccgcttgaaagacaaagttaacaataccctgagcaccgacatcccatttcaactgagcaagtatgttgataatcaacgtctgttgagcactttcaccgagtatatcaaaaacatcatcaatactagcattctgaacctgcgttacgagagcaagcatctgattgatctgagccgttatgctagcaagatcaacatcggtagcaaggtcaattttgacccgatcgataagaaccagatccagctgtttaatctggaatcgagcaaaattgaggttatcctgaaaaaggccattgtctacaactccatgtacgagaatttctccaccagcttctggattcgcatcccgaaatacttcaacaagattagcctgaacaacgagtatactatcatcaactgtatggagaacaacagcggttggaaggtgtctctgaactatggtgagatcatttggaccttgcaggacaccaaagagatcaagcagcgcgtcgtgttcaagtactctcaaatgatcaacatttccgattacattaatcgttggatcttcgtgaccattacgaataaccgtctgaataagagcaagatttacatcaatggtcgcttgatcgatcagaaaccgattagcaacctgggtaatatccacgcaagcaacaagattatgttcaaattggacggttgccgcgatacccatcgttatatctggatcaagtatttcaacctgtttgataaagaactgaatgagaaggagatcaaagatttgtatgacaaccaatctaacagcggcattttgaaggacttctggggcgattatctgcaatacgataagccgtactatatgctgaacctgtatgatccgaacaaatatgtggatgtcaataatgtgggtattcgtggttacatgtatttgaagggtccgcgtggcagcgttatgacgaccaacatttacctgaactctagcctgtaccgtggtacgaaattcatcattaagaaatatgccagcggcaacaaagataacattgtgcgtaataacgatcgtgtctacatcaacgtggtcgtgaagaataaagagtaccgtctggcgaccaacgcttcgcaggcgggtgttgagaaaattctgagcgcgttggagatccctgatgtcggtaatctgagccaagtcgtggttatgaagagcaagaacgacaagggtatcactaacaagtgcaagatgaacctgcaagacaacaatggtaacgacatcggctttattggtttccaccagttcaacaatattgctaaactggtagcgagcaattggtacaatcgtcagattgagcgcagcagccgtactttgggctgtagctgggagtttatcccggtcgatgatggttggggcgaacgtccgctg

SEQ ID NO: 4 - protein, 1296 aa, Artificial
  Modified BoNT/A "Cat-A"
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIINTSILNLRYESKHLIDLSRYASKINIGSKVNFDPIDKNQIQLFNLESSKIEVILKKAIVYNSMYENFSTSFWIRIPKYFNKISLNNEYTIINCMENNSGWKVSLNYGEIIWTLQDTKEIKQRVVFKYSQMINISDYINRWIFVTITNNRLNKSKIYINGRLIDQKPISNLGNIHASNKIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDKGITNKCKMNLQDNNGNDIGFIGFHQFNNIAKLVASNWYNRQIERSSRTLGCSWEFIPVDDGWGERPL

SEQ ID NO: 5 - DNA, 3888 bp, Artificial
  Modified BoNT/A "Cat-B"
atgccattcgtcaacaagcaattcaactacaaagacccagtcaacggcgtcgacatcgcatacatcaagattccgaacgccggtcaaatgcagccggttaaggcttttaagatccacaacaagatttgggttatcccggagcgtgacaccttcacgaacccggaagaaggcgatctgaacccgccaccggaagcgaagcaagtccctgtcagctactacgattcgacgtacctgagcacggataacgaaaaagataactacctgaaaggtgtgaccaagctgttcgaacgtatctacagcacggatctgggtcgcatgctgctgactagcattgttcgcggtatcccgttctggggtggtagcacgattgacaccgaactgaaggttatcgacactaactgcattaacgttattcaaccggatggtagctatcgtagcgaagagctgaatctggtcatcattggcccgagcgcagacattatccaattcgagtgcaagagctttggtcacgaggttctgaatctgacccgcaatggctatggtagcacccagtacattcgtttttcgccggattttaccttcggctttgaagagagcctggaggttgataccaatccgttgctgggtgcgggcaaattcgctaccgatccggctgtcacgctggcccatgaactgatccacgcaggccaccgcctgtacggcattgccatcaacccaaaccgtgtgttcaaggttaatacgaatgcatactacgagatgagcggcctggaagtcagcttcgaagaactgcgcaccttcggtggccatgacgctaaattcattgacagcttgcaagagaatgagttccgtctgtactactataacaaattcaaagacattgcaagcacgttgaacaaggccaaaagcatcgttggtactaccgcgtcgttgcagtatatgaagaatgtgtttaaagagaagtacctgctgtccgaggatacctccggcaagtttagcgttgataagctgaagtttgacaaactgtacaagatgctgaccgagatttacaccgaggacaactttgtgaaattcttcaaagtgttgaatcgtaaaacctatctgaattttgacaaagcggttttcaagattaacatcgtgccgaaggtgaactacaccatctatgacggttttaacctgcgtaacaccaacctggcggcgaactttaacggtcagaatacggaaatcaacaacatgaatttcacgaagttgaagaacttcacgggtctgttcgagttctataagctgctgtgcgtgcgcggtatcatcaccagcaaaaccaaaagcctggacaaaggctacaacaaggcgctgaatgacctgtgcattaaggtaaacaattgggatctgttcttttcgccatccgaagataattttaccaacgacctgaacaagggtgaagaaatcaccagcgatacgaatattgaagcagcggaagagaatatcagcctggatctgatccagcagtactatctgacctttaacttcgacaatgaaccggagaacattagcattgagaatctgagcagcgacattatcggtcagctggaactgatgccgaatatcgaacgtttcccgaacggcaaaaagtacgagctggacaagtacactatgttccattacctgcgtgcacaggagtttgaacacggtaaaagccgtatcgcgctgaccaacagcgttaacgaggccctgctgaacccgagccgtgtctataccttcttcagcagcgactatgttaagaaagtgaacaaagccactgaggccgcgatgttcctgggctgggtggaacagctggtatatgacttcacggacgagacgagcgaagtgagcactaccgacaaaattgctgatattaccatcattatcccgtatattggtccggcactgaacattggcaacatgctgtacaaagacgattttgtgggtgccctgatcttctccggtgccgtgattctgctggagttcattccggagattgcgatcccggtgttgggtaccttcgcgctggtgtcctacatcgcgaataaggttctgacggttcagaccatcgataacgcgctgtcgaaacgtaatgaaaaatgggacgaggtttacaaatacattgttacgaattggctggcgaaagtcaatacccagatcgacctgatccgtaagaaaatgaaagaggcgctggagaatcaggcggaggccaccaaagcaattatcaactaccaatacaaccagtacacggaagaagagaagaataacattaacttcaatatcgatgatttgagcagcaagctgaatgaatctatcaacaaagcgatgatcaatatcaacaagtttttgaatcagtgtagcgtttcgtacctgatgaatagcatgattccgtatggcgtcaaacgtctggaggacttcgacgccagcctgaaagatgcgttgctgaaatacatttacgacaatcgtggtacgctgattggccaagttgaccgcttgaaagacaaagttaacaataccctgagcaccgacatcccatttcaactgagcaagtatgttgataatcaacgtctgttgagcactttcaccgagtatatcaaaaacatcatcaatactagcattctgaacctgcgttacgagagcaatcatctgattgatctgagccgttatgctagcaagatcaacatcggtagcaaggtcaattttgacccgatcgataagaaccagatccagctgtttaatctggaatcgagcaaaattgaggttatcctgaaaaaggccattgtctacaactccatgtacgagaatttctccaccagcttctggattcgcatcccgaaatacttcaagaagattagcctgaacaacgagtatactatcatcaactgtatggagaacaacagcggttggaaggtgtctctgaactatggtgagatcatttggaccttgcaggacaccaaagagatcaagcagcgcgtcgtgttcaagtactctcaaatgatcaacatttccgattacattaatcgttggatcttcgtgaccattacgaataaccgtctgaataagagcaagatttacatcaatggtcgcttgatcgatcagaaaccgattagcaacctgggtaatatccacgcaagcaacaagattatgttcaaattggacggttgccgcgatacccatcgttatatctggatcaagtatttcaacctgtttgataaagaactgaatgagaaggagatcaaagatttgtatgacaaccaatctaacagcggcattttgaaggacttctggggcgattatctgcaatacgataagccgtactatatgctgaacctgtatgatccgaacaaatatgtggatgtcaataatgtgggtattcgtggttacatgtatttgaagggtccgcgtggcagcgttatgacgaccaacatttacctgaactctagcctgtaccgtggtacgaaattcatcattaagaaatatgccagcggcaacaaagataacattgtgcgtaataacgatcgtgtctacatcaacgtggtcgtgaagaataaagagtaccgtctggcgaccaacgcttcgcaggcgggtgttgagaaaattctgagcgcgttggagatccctgatgtcggtaatctgagccaagtcgtggttatgaagagcaagaacgacaagggtatcactaacaagtgcaagatgaacctgcaagacaacaatggtaacgacatcggctttattggtttccaccagttcaacaatattgctaaactggtagcgagcaattggtacaatcgtcagattgagcgcagcagccgtactttgggctgtagctgggagtttatcccggtcgatgatggttggggcgaacgtccgctg

SEQ ID NO: 6 - protein, 1296 aa, artificial
  Modified BoNT/A "Cat-B"
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIINTSILNLRYESNHLIDLSRYASKINIGSKVNFDPIDKNQIQLFNLESSKIEVILKKAIVYNSMYENFSTSFWIRIPKYFKKISLNNEYTIINCMENNSGWKVSLNYGEIIWTLQDTKEIKQRVVFKYSQMINISDYINRWIFVTITNNRLNKSKIYINGRLIDQKPISNLGNIHASNKIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDKGITNKCKMNLQDNNGNDIGFIGFHQFNNIAKLVASNWYNRQIERSSRTLGCSWEFIPVDDGWGERPL

SEQ ID NO: 7 - DNA, 3888 bp, artificial
  Modified BoNT/A "Cat-C"
atgccattcgtcaacaagcaattcaactacaaagacccagtcaacggcgtcgacatcgcatacatcaagattccgaacgccggtcaaatgcagccggttaaggcttttaagatccacaacaagatttgggttatcccggagcgtgacaccttcacgaacccggaagaaggcgatctgaacccgccaccggaagcgaagcaagtccctgtcagctactacgattcgacgtacctgagcacggataacgaaaaagataactacctgaaaggtgtgaccaagctgttcgaacgtatctacagcacggatctgggtcgcatgctgctgactagcattgttcgcggtatcccgttctggggtggtagcacgattgacaccgaactgaaggttatcgacactaactgcattaacgttattcaaccggatggtagctatcgtagcgaagagctgaatctggtcatcattggcccgagcgcagacattatccaattcgagtgcaagagctttggtcacgaggttctgaatctgacccgcaatggctatggtagcacccagtacattcgtttttcgccggattttaccttcggctttgaagagagcctggaggttgataccaatccgttgctgggtgcgggcaaattcgctaccgatccggctgtcacgctggcccatgaactgatccacgcaggccaccgcctgtacggcattgccatcaacccaaaccgtgtgttcaaggttaatacgaatgcatactacgagatgagcggcctggaagtcagcttcgaagaactgcgcaccttcggtggccatgacgctaaattcattgacagcttgcaagagaatgagttccgtctgtactactataacaaattcaaagacattgcaagcacgttgaacaaggccaaaagcatcgttggtactaccgcgtcgttgcagtatatgaagaatgtgtttaaagagaagtacctgctgtccgaggatacctccggcaagtttagcgttgataagctgaagtttgacaaactgtacaagatgctgaccgagatttacaccgaggacaactttgtgaaattcttcaaagtgttgaatcgtaaaacctatctgaattttgacaaagcggttttcaagattaacatcgtgccgaaggtgaactacaccatctatgacggttttaacctgcgtaacaccaacctggcggcgaactttaacggtcagaatacggaaatcaacaacatgaatttcacgaagttgaagaacttcacgggtctgttcgagttctataagctgctgtgcgtgcgcggtatcatcaccagcaaaaccaaaagcctggacaaaggctacaacaaggcgctgaatgacctgtgcattaaggtaaacaattgggatctgttcttttcgccatccgaagataattttaccaacgacctgaacaagggtgaagaaatcaccagcgatacgaatattgaagcagcggaagagaatatcagcctggatctgatccagcagtactatctgacctttaacttcgacaatgaaccggagaacattagcattgagaatctgagcagcgacattatcggtcagctggaactgatgccgaatatcgaacgtttcccgaacggcaaaaagtacgagctggacaagtacactatgttccattacctgcgtgcacaggagtttgaacacggtaaaagccgtatcgcgctgaccaacagcgttaacgaggccctgctgaacccgagccgtgtctataccttcttcagcagcgactatgttaagaaagtgaacaaagccactgaggccgcgatgttcctgggctgggtggaacagctggtatatgacttcacggacgagacgagcgaagtgagcactaccgacaaaattgctgatattaccatcattatcccgtatattggtccggcactgaacattggcaacatgctgtacaaagacgattttgtgggtgccctgatcttctccggtgccgtgattctgctggagttcattccggagattgcgatcccggtgttgggtaccttcgcgctggtgtcctacatcgcgaataaggttctgacggttcagaccatcgataacgcgctgtcgaaacgtaatgaaaaatgggacgaggtttacaaatacattgttacgaattggctggcgaaagtcaatacccagatcgacctgatccgtaagaaaatgaaagaggcgctggagaatcaggcggaggccaccaaagcaattatcaactaccaatacaaccagtacacggaagaagagaagaataacattaacttcaatatcgatgatttgagcagcaagctgaatgaatctatcaacaaagcgatgatcaatatcaacaagtttttgaatcagtgtagcgtttcgtacctgatgaatagcatgattccgtatggcgtcaaacgtctggaggacttcgacgccagcctgaaagatgcgttgctgaaatacatttacgacaatcgtggtacgctgattggccaagttgaccgcttgaaagacaaagttaacaataccctgagcaccgacatcccatttcaactgagcaagtatgttgataatcaacgtctgttgagcactttcaccgagtatatcaaaaacatcatcaatactagcattctgaacctgcgttacgagagcaatcatctgattgatctgagccgttatgctagcaagatcaacatcggtagcaaggtcaattttgacccgatcgataagaaccagatccagctgtttaatctggaatcgagcaaaattgaggttatcctgaaaaaggccattgtctacaactccatgtacgagaatttctccaccagcttctggattcgcatcccgaaatacttcaacaagattagcctgaacaacgagtatactatcatcaactgtatggagaacaacagcggttggaaggtgtctctgaactatggtgagatcatttggaccttgcaggacaccaaagagatcaagcagcgcgtcgtgttcaagtactctcaaatgatcaacatttccgattacattaatcgttggatcttcgtgaccattacgaataaccgtctgaagaagagcaagatttacatcaatggtcgcttgatcgatcagaaaccgattagcaacctgggtaatatccacgcaagcaacaagattatgttcaaattggacggttgccgcgatacccatcgttatatctggatcaagtatttcaacctgtttgataaagaactgaatgagaaggagatcaaagatttgtatgacaaccaatctaacagcggcattttgaaggacttctggggcgattatctgcaatacgataagccgtactatatgctgaacctgtatgatccgaacaaatatgtggatgtcaataatgtgggtattcgtggttacatgtatttgaagggtccgcgtggcagcgttatgacgaccaacatttacctgaactctagcctgtaccgtggtacgaaattcatcattaagaaatatgccagcggcaacaaagataacattgtgcgtaataacgatcgtgtctacatcaacgtggtcgtgaagaataaagagtaccgtctggcgaccaacgcttcgcaggcgggtgttgagaaaattctgagcgcgttggagatccctgatgtcggtaatctgagccaagtcgtggttatgaagagcaagaacgacaagggtatcactaacaagtgcaagatgaacctgcaagacaacaatggtaacgacatcggctttattggtttccaccagttcaacaatattgctaaactggtagcgagcaattggtacaatcgtcagattgagcgcagcagccgtactttgggctgtagctgggagtttatcccggtcgatgatggttggggcgaacgtccgctg

SEQ ID NO: 8 - protein, 1296 aa, artificial
  Modified BoNT/A "Cat-C"
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIINTSILNLRYESNHLIDLSRYASKINIGSKVNFDPIDKNQIQLFNLESSKIEVILKKAIVYNSMYENFSTSFWIRIPKYFNKISLNNEYTIINCMENNSGWKVSLNYGEIIWTLQDTKEIKQRVVFKYSQMINISDYINRWIFVTITNNRLKKSKIYINGRLIDQKPISNLGNIHASNKIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDKGITNKCKMNLQDNNGNDIGFIGFHQFNNIAKLVASNWYNRQIERSSRTLGCSWEFIPVDDGWGERPL

SEQ ID NO: 9 - DNA, 3888 bp, artificial
  Modified BoNT/A "Cat-D"
atgccattcgtcaacaagcaattcaactacaaagacccagtcaacggcgtcgacatcgcatacatcaagattccgaacgccggtcaaatgcagccggttaaggcttttaagatccacaacaagatttgggttatcccggagcgtgacaccttcacgaacccggaagaaggcgatctgaacccgccaccggaagcgaagcaagtccctgtcagctactacgattcgacgtacctgagcacggataacgaaaaagataactacctgaaaggtgtgaccaagctgttcgaacgtatctacagcacggatctgggtcgcatgctgctgactagcattgttcgcggtatcccgttctggggtggtagcacgattgacaccgaactgaaggttatcgacactaactgcattaacgttattcaaccggatggtagctatcgtagcgaagagctgaatctggtcatcattggcccgagcgcagacattatccaattcgagtgcaagagctttggtcacgaggttctgaatctgacccgcaatggctatggtagcacccagtacattcgtttttcgccggattttaccttcggctttgaagagagcctggaggttgataccaatccgttgctgggtgcgggcaaattcgctaccgatccggctgtcacgctggcccatgaactgatccacgcaggccaccgcctgtacggcattgccatcaacccaaaccgtgtgttcaaggttaatacgaatgcatactacgagatgagcggcctggaagtcagcttcgaagaactgcgcaccttcggtggccatgacgctaaattcattgacagcttgcaagagaatgagttccgtctgtactactataacaaattcaaagacattgcaagcacgttgaacaaggccaaaagcatcgttggtactaccgcgtcgttgcagtatatgaagaatgtgtttaaagagaagtacctgctgtccgaggatacctccggcaagtttagcgttgataagctgaagtttgacaaactgtacaagatgctgaccgagatttacaccgaggacaactttgtgaaattcttcaaagtgttgaatcgtaaaacctatctgaattttgacaaagcggttttcaagattaacatcgtgccgaaggtgaactacaccatctatgacggttttaacctgcgtaacaccaacctggcggcgaactttaacggtcagaatacggaaatcaacaacatgaatttcacgaagttgaagaacttcacgggtctgttcgagttctataagctgctgtgcgtgcgcggtatcatcaccagcaaaaccaaaagcctggacaaaggctacaacaaggcgctgaatgacctgtgcattaaggtaaacaattgggatctgttcttttcgccatccgaagataattttaccaacgacctgaacaagggtgaagaaatcaccagcgatacgaatattgaagcagcggaagagaatatcagcctggatctgatccagcagtactatctgacctttaacttcgacaatgaaccggagaacattagcattgagaatctgagcagcgacattatcggtcagctggaactgatgccgaatatcgaacgtttcccgaacggcaaaaagtacgagctggacaagtacactatgttccattacctgcgtgcacaggagtttgaacacggtaaaagccgtatcgcgctgaccaacagcgttaacgaggccctgctgaacccgagccgtgtctataccttcttcagcagcgactatgttaagaaagtgaacaaagccactgaggccgcgatgttcctgggctgggtggaacagctggtatatgacttcacggacgagacgagcgaagtgagcactaccgacaaaattgctgatattaccatcattatcccgtatattggtccggcactgaacattggcaacatgctgtacaaagacgattttgtgggtgccctgatcttctccggtgccgtgattctgctggagttcattccggagattgcgatcccggtgttgggtaccttcgcgctggtgtcctacatcgcgaataaggttctgacggttcagaccatcgataacgcgctgtcgaaacgtaatgaaaaatgggacgaggtttacaaatacattgttacgaattggctggcgaaagtcaatacccagatcgacctgatccgtaagaaaatgaaagaggcgctggagaatcaggcggaggccaccaaagcaattatcaactaccaatacaaccagtacacggaagaagagaagaataacattaacttcaatatcgatgatttgagcagcaagctgaatgaatctatcaacaaagcgatgatcaatatcaacaagtttttgaatcagtgtagcgtttcgtacctgatgaatagcatgattccgtatggcgtcaaacgtctggaggacttcgacgccagcctgaaagatgcgttgctgaaatacatttacgacaatcgtggtacgctgattggccaagttgaccgcttgaaagacaaagttaacaataccctgagcaccgacatcccatttcaactgagcaagtatgttgataatcaacgtctgttgagcactttcaccgagtatatcaaaaacatcatcaatactagcattctgaacctgcgttacgagagcaatcatctgattgatctgagccgttatgcaagcaagatcaacatcggtagcaaggtcaattttgacccgatcgataagaaccagatccagctgtttaatctggaatcgagcaaaattgaggttatcctgaaaaacgccattgtctacaactccatgtacgagaatttctccaccagcttctggattcgcatcccgaaatacttcaacagcattagcctgaacaacgagtatactatcatcaactgtatggagaacaacagcggttggaaggtgtctctgaactatggtgagatcatttggaccttgcaggacacccaagagatcaagcagcgcgtcgtgttcaagtactctcaaatgatcaacatttccgattacattaatcgttggatcttcgtgaccattacgaataaccgtctgaataacagcaagatttacatcaatggtcgcttgatcgatcagaaaccgattagcaacctgggtaatatccacgcaagcaacaacattatgttcaaattggacggttgccgcgatacccatcgttatatctggatcaagtatttcaacctgtttgataaagaactgaatgagaaggagatcaaagatttgtatgacaaccaatctaacagcggcattttgaaggacttctggggcgattatctgcaatacgataagccgtactatatgctgaacctgtatgatccgaacaaatatgtggatgtcaataatgtgggtattcgtggttacatgtatttgaagggtccgcgtggcagcgttatgacgaccaacatttacctgaactctagcctgtaccgtggtacgaaattcatcattaagaaatatgccagcggcaacaaagataacattgtgcgtaataacgatcgtgtctacatcaacgtggtcgtgaagcgtaaagagtaccgtctggcgaccaacgcttcgcaggcgggtgttgagaaaattctgagcgcgttggagatccctcgtgtccgtcgtctgagccaagtcgtggttatgaagagcaagaacgaccagggtatcactaacaagtgcaagatgaacctgcaagaccgtcgtggtaacgacatcggctttattggtttccaccagttcaacaatattgctaaactggtagcgagcaattggtacaatcgtcagattgagcgccgtagccgtcgtttgggctgtagctgggagtttatcccggtcgatgatggttggggcgaacgtccgctg

SEQ ID NO: 10 - protein, 1296 aa, artificial
  Modified BoNT/A "Cat-D"
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIINTSILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKRKEYRLATNASQAGVEKILSALEIPRVRRLSQVVVMKSKNDQGITNKCKMNLQDRRGNDIGFIGFHQFNNIAKLVASNWYNRQIERRSRRLGCSWEFIPVDDGWGERPL

SEQ ID NO: 11 - protein, 1315 aa, Artificial Sequence
  Modified BoNT/A "Chimera 1"
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKSEILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEMKLFLAPIYDSDEFYNTIQIKEYDEQPTYSCQLLFKKDEESTDEIGLIGIHRFYESGIVFEEYKDYFCISKWYLKEVKRKPYNLKLGCNWQFIPKDEGWTEHHHHHHHHHH

SEQ ID NO: 12 - protein, 1327 aa, Artificial Sequence
  Modified BoNT/A "Chimera 2"
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIIELGGGGSELSEILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEMKLFLAPIYDSDEFYNTIQIKEYDEQPTYSCQLLFKKDEESTDEIGLIGIHRFYESGIVFEEYKDYFCISKWYLKEVKRKPYNLKLGCNWQFIPKDEGWTEHHHHHHHHHH

SEQ ID NO: 13 - protein, 1314 aa, Artificial Sequence
  Modified BoNT/A "Chimera 3A"
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEMKLFLAPIYDSDEFYNTIQIKEYDEQPTYSCQLLFKKDEESTDEIGLIGIHRFYESGIVFEEYKDYFCISKWYLKEVKRKPYNLKLGCNWQFIPKDEGWTEHHHHHHHHHH

SEQ ID NO: 14 - protein, 1304 aa, Artificial Sequence
  Modified BoNT/A "Chimera 3B"
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEMKLFLAPIYDSDEFYNTIQIKEYDEQPTYSCQLLFKKDEESTDEIGLIGIHRFYESGIVFEEYKDYFCISKWYLKEVKRKPYNLKLGCNWQFIPKDEGWTE

SEQ ID NO: 15 - protein, 1304 aa, Artificial Sequence
  Modified BoNT/A "Chimera 3C"
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEEKLFLAPISDSDEFYNTIQIKEYDEQPTYSCQLLFKKDEESTDEIGLIGIHRFYESGIVFEEYKDYFCISKWYLKEVKRKPYNLKLGCNWQFIPKDEGWTE

SEQ ID NO: 16 - protein, 1291 aa, Clostridium botulinum
MPVTINNFNYNDPIDNNNIIMMEPPFARGTGRYYKAFKITDRIWIIPERYTFGYKPEDFNKSSGIFNRDVCEYYDPDYLNTNDKKNIFLQTMIKLFNRIKSKPLGEKLLEMIINGIPYLGDRRVPLEEFNTNIASVTVNKLISNPGEVERKKGIFANLIIFGPGPVLNENETIDIGIQNHFASREGFGGIMQMKFCPEYVSVFNNVQENKGASIFNRRGYFSDPALILMHELIHVLHGLYGIKVDDLPIVPNEKKFFMQSTDAIQAEELYTFGGQDPSIITPSTDKSIYDKVLQNFRGIVDRLNKVLVCISDPNININIYKNKFKDKYKFVEDSEGKYSIDVESFDKLYKSLMFGFTETNIAENYKIKTRASYFSDSLPPVKIKNLLDNEIYTIEEGFNISDKDMEKEYRGQNKAINKQAYEEISKEHLAVYKIQMCKSVKAPGICIDVDNEDLFFIADKNSFSDDLSKNERIEYNTQSNYIENDFPINELILDTDLISKIELPSENTESLTDFNVDVPVYEKQPAIKKIFTDENTIFQYLYSQTFPLDIRDISLTSSFDDALLFSNKVYSFFSMDYIKTANKVVEAGLFAGWVKQIVNDFVIEANKSNTMDKIADISLIVPYIGLALNVGNETAKGNFENAFEIAGASILLEFIPELLIPVVGAFLLESYIDNKNKIIKTIDNALTKRNEKWSDMYGLIVAQWLSTVNTQFYTIKEGMYKALNYQAQALEEIIKYRYNIYSEKEKSNINIDFNDINSKLNEGINQAIDNINNFINGCSVSYLMKKMIPLAVEKLLDFDNTLKKNLLNYIDENKLYLIGSAEYEKSKVNKYLKTIMPFDLSIYTNDTILIEMFNKYNSEILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEEKLFLAPISDSDEFYNTIQIKEYDEQPTYSCQLLFKKDEESTDEIGLIGIHRFYESGIVFEEYKDYFCISKWYLKEVKRKPYNLKLGCNWQFIPKDEGWTE

(19) United States
(12) Patent Application Publication
(10) Pub. No.: US 2023/0346674 A1
(43) Pub. Date: Nov. 2, 2023

(54) Treatment of Upper Facial Lines

(71) Applicant: IPSEN BIOPHARM LIMITED, Wrexham (GB)

(72) Inventors: Nicolae GRIGORE, Wrexham (GB); Katie RUFFLE, Wrexham (GB); Philippe PICAUT, Wrexham (GB)

(21) Appl. No.: 17/911,503

(22) PCT Filed: Mar. 16, 2021
(86) PCT No.: PCT/GB2021/050659

§ 371 Date: Sep. 14, 2022

(30) Foreign Application Priority Data
Mar. 16, 2020 (GB) 2003813.9

(51) Int. Cl. A61K 9/00; A61K 38/48; A61Q 19/08; A61K 8/66

(52) U.S. Cl. CPC A61K 8/66; A61K 9/0019; A61K 38/4893; A61Q 19/08; A61K 2800/74; A61K 2800/91

(57) ABSTRACT

The present invention relates to a modified BoNT/A for use in treating facial lines, wherein the modified BoNT/A is administered by intramuscular injection at a plurality of sites of the face of the subject, wherein the modified BoNT/A is administered by way of a unit dose of Units to 41 Units of modified BoNT/A per site and wherein 1 Unit is an amount of the modified BoNT/A that corresponds to the calculated median lethal dose (LD₅₀) in mice, wherein the plurality of sites are selected from: up to two sites of a corrugator muscle and one site of a procerus muscle for treating glabellar lines, up to five sites of a frontalis muscle for treating forehead lines; and up to three sites at the external part of an orbicularis oculi muscle for treating lateral canthal lines, wherein the total dose of modified BoNT/A administered during the treatment is up to 574 Units, and wherein the modified BoNT/A comprises a modification at one or more amino acid residue(s) selected from: ASN 886, ASN 905, GLN 915, ASN 918, GLU 920, ASN 930, ASN 954, SER 955, GLN 991, GLU 992, GLN 995, ASN 1006, ASN 1025, ASN 1026, ASN 1032, ASN 1043, ASN 1046, ASN 1052, ASP 15 1058, HIS 1064, ASN 1080, GLU 1081, GLU 1083, ASP 1086, ASN 1188, ASP 1213, GLY 1215, ASN 1216, GLN 1229, ASN 1242, ASN 1243, SER 1274, and THR 1277, wherein the modification is selected from: i. substitution of an acidic surface exposed amino acid residue with a basic amino acid residue; ii. substitution of an acidic surface exposed amino acid residue with an uncharged amino acid residue; iii. substitution of an uncharged surface exposed amino acid residue with a basic amino acid residue; iv. insertion of a basic amino acid residue; and v. deletion of an acidic surface exposed amino acid residue. Also provided are corresponding methods of treatment and uses, as well as unit dosage forms, and kits.

### FIELD OF THE INVENTION

The present invention relates to cosmetic treatment (e.g. for treating hyperfunctional facial lines). In more detail, the invention provides methods of cosmetic treatment comprising administration of longer acting neurotoxins and more particularly, to a method of treating cosmetic irregularities using longer acting botulinum neurotoxins.

### BACKGROUND

Hyperfunctional facial lines are common aesthetic irregularities that can include but are not limited to; glabellar lines, lateral canthal lines, forehead lines and wrinkles, crow’s feet, eyebrow lines, nasolabial folds, lip lines and marionette lines. Upper facial lines can occur in the forehead, glabellar and the lateral orbital areas. Wrinkling may also appear in the glabellar and forehead areas due to the expression of frowning, whilst wrinkling may appear in the lateral canthal areas due to the expression of smiling. Excessively prominent lines in this area are often misinterpreted as fatigue causing subjects considerable distress regarding their appearance. Instead, excessively prominent facial lines appear as a result from the functional pull of the underlying muscles, which eventually creases the skin. In the glabellar complex, these muscles include the corrugator supercilli, the procerus and the depressor supercilli, whilst the orbicularis oculi muscle is responsible for the production of lateral canthal lines.

Over the last few decades, as the aging population continues to grow, there has been an increasing demand for cosmetic procedures to reverse the appearance of advancing age, particularly in the facial area. This increasing preoccupation with physical appearance has led to the development of many different products and procedures, such as surgery, resurfacing of various types and the use of filling agents. Because none of these methods are entirely risk free, continuous research is necessary to provide the safest and most effective methods for treatment of the aging face.

Although there are five factors that interplay in the production of what is known as the aging face, there are primarily two factors (the skin and the underlying muscles) interacting to produce the more significant lines and folds, as opposed to wrinkles. Many therapies have evolved to treat wrinkles and the skin factors of the lines and folds, among them, various types of resurfacing, dermatologic products and injections for soft tissue augmentation.

An alternative cosmetic procedure for treating the appearance of facial lines is the administration of neurotoxin, specifically botulinum neurotoxin, to the underlying muscles of facial skin. Dysport® is a medicinal product containing drug substance BoNT/A haemagglutinin complex (BTX-A-HAC) isolated and purified from Clostridium botulinum type A strain. Several other medicinal BoNT/A products naturally produced by Clostridium botulinum are also on the market.

BoNT/A selectively inhibits the release of acetylcholine from the presynaptic nerve terminals and thus blocks cholinergic transmission at the neuromuscular junction inducing a reduction in the muscle contraction and muscle tone, causing the injected muscles to relax.

Dysport® is approved for the treatment of glabellar lines with a maximum total dose of up to 50 Units to be administered across the corrugator muscles and procerus muscle (see FIG. 1). The clinical effect of Dysport may last up to four months. Repeat dosing in clinical studies demonstrated continued efficacy with up to four repeated administrations. However, Dysport should be administered no more frequently than every three months. Dysport has so far not been FDA approved for the treatment of forehead lines and lateral canthal lines.

To avoid systemic neurological effects, many clostridial toxin based cosmetic treatments utilise direct administration of the clostridial toxin therapeutic to a given target site (such as a target tissue). A problem when administering clostridial toxin-based therapeutics in this fashion is the spread of toxin away from the administration site and into surrounding tissue or systemic circulation. The diffusion of toxin away from the target tissue is believed to be responsible for undesirable side effects that in extreme cases may be life threatening. This can be a particular concern when using clostridial toxin therapeutics (such as BoNT therapeutics) at high doses, concentrations and injection volumes. Adverse effects associated with this problem that have been reported for commercial BoNT/A therapeutics include asthenia, generalised muscle weakness, diplopia, ptosis, dysphagia, dysphonia, dysarthria, urinary incontinence, and breathing difficulties. Swallowing and breathing difficulties can be life threatening and there have been reported deaths related to the spread of toxin effects.

The present invention overcomes one or more of the above-mentioned problems.

### SUMMARY OF THE INVENTION

The present inventors have surprisingly found that a modified BoNT/A finds particular utility in cosmetic treatment of facial lines. The modified BoNT/A may comprise one or more modifications of surface exposed amino acid residues resulting in an increased net positive charge. The increased charge promotes electrostatic interactions between the polypeptide and anionic extracellular components, thereby promoting binding between the polypeptide and cell surface. In turn this increases retention at (reduces diffusion away from) a site of administration and results in an increased duration of action (e.g. 6-9 months). Alternatively, a modified BoNT/A may comprise a BoNT/A light-chain and translocation domain and a BoNT/B receptor binding domain (H_{c} domain), which similarly results in a modified BoNT/A that exhibits increased retention at (reduced diffusion away from) a site of administration and increased duration of action (e.g. 6-9 months). Advantageously, modified BoNT/A has a safety profile that is improved when compared to unmodified BoNT/A (e.g. Dysport®). This improved safety profile may be expressed by the high Safety Ratio described herein for the modified BoNT/A.

Based on the pre-clinical data herein (see Example 6) it has been shown that a higher total amount of modified BoNT/A can be administered to a subject while achieving a similar safety profile to unmodified BoNT/A (e.g. Dysport®) while at such high doses. Thus, modified BoNT/A can be injected at a greater number of muscles/sites in the cosmetic treatment of facial lines before reaching the maximum total dose. This is a significant and advantageous finding and yields an improved cosmetic treatment of facial lines while providing clinicians with a greater range of treatment options. The treatment may be improved in that it provides for longer-lasting treatment (resulting in less frequent administration) when compared to treatment with unmodified BoNT/A (e.g. Dysport®) and/or is capable of being tailored for the subject, for example, enabling the clinician to administer at particular sites according to the subject’s aesthetic requirements. The treatment of the invention is improved compared to conventional treatment regimens.

Moreover, the present invention provides a convenient, safe, and effective single dose unit as well as a total (maximum) dosage that can safely be administered in a single treatment. The present invention also provides a corresponding guide to the number of times at which said dose unit can be administered to a muscle (including the number of injection sites per muscle) without resultant patient toxicity. Treatment in accordance with the present invention is thus much less complicated for the clinician and helps avoid under-dosing and/or overdosing.

### DETAILED DESCRIPTION

In one aspect the invention provides a modified BoNT/A for use in treating facial lines, wherein the modified BoNT/A is administered by intramuscular injection at a plurality of sites of the face of the subject,
wherein the modified BoNT/A is administered by way of a unit dose of 1 Units to 41 Units (2 Units to 41 Units) of modified BoNT/A per site and wherein 1 Unit is an amount of the modified BoNT/A that corresponds to the calculated median lethal dose (LD₅₀) in mice,
wherein the plurality of sites are selected from:
   up to two sites of a corrugator muscle and one site of a procerus muscle for treating glabellar lines,
   up to five sites of a frontalis muscle for treating forehead lines; and
   up to three sites at the external part of a orbicularis oculi muscle for treating lateral canthal lines,
wherein the total dose of modified BoNT/A administered during the treatment is up to 574 Units, and
wherein the modified BoNT/A comprises a modification at one or more amino acid residue(s) selected from: ASN 886, ASN 905, GLN 915, ASN 918, GLU 920, ASN 930, ASN 954, SER 955, GLN 991, GLU 992, GLN 995, ASN 1006, ASN 1025, ASN 1026, ASN 1032, ASN 1043, ASN 1046, ASN 1052, ASP 1058, HIS 1064, ASN 1080, GLU 1081, GLU 1083, ASP 1086, ASN 1188, ASP 1213, GLY 1215, ASN 1216, GLN 1229, ASN 1242, ASN 1243, SER 1274, and THR 1277, wherein the modification is selected from:
   i. substitution of an acidic surface exposed amino acid residue with a basic amino acid residue;
   ii. substitution of an acidic surface exposed amino acid residue with an uncharged amino acid residue;
   iii. substitution of an uncharged surface exposed amino acid residue with a basic amino acid residue;
   iv. insertion of a basic amino acid residue; and
   v. deletion of an acidic surface exposed amino acid residue.

In another related aspect, the invention provides a modified BoNT/A for use in treating facial lines, wherein the modified BoNT/A is administered by intramuscular injection at a plurality of sites of the face of the subject,
wherein the modified BoNT/A is administered by way of a unit dose of 8.4 pg to 344.4 pg of modified BoNT/A per site,
wherein the plurality of sites are selected from:
   up to two sites of a corrugator muscle and one site of a procerus muscle for treating glabellar lines,
   up to five sites of a frontalis muscle for treating forehead lines; and
   up to three sites at the external part of an orbicularis oculi muscle for treating lateral canthal lines,
wherein the total dose of modified BoNT/A administered during the treatment is up to 4821.6 pg, and
wherein the modified BoNT/A comprises a modification at one or more amino acid residue(s) selected from: ASN 886, ASN 905, GLN 915, ASN 918, GLU 920, ASN 930, ASN 954, SER 955, GLN 991, GLU 992, GLN 995, ASN 1006, ASN 1025, ASN 1026, ASN 1032, ASN 1043, ASN 1046, ASN 1052, ASP 1058, HIS 1064, ASN 1080, GLU 1081, GLU 1083, ASP 1086, ASN 1188, ASP 1213, GLY 1215, ASN 1216, GLN 1229, ASN 1242, ASN 1243, SER 1274, and THR 1277, wherein the modification is selected from:
   i. substitution of an acidic surface exposed amino acid residue with a basic amino acid residue;
   ii. substitution of an acidic surface exposed amino acid residue with an uncharged amino acid residue;
   iii. substitution of an uncharged surface exposed amino acid residue with a basic amino acid residue;
   iv. insertion of a basic amino acid residue; and
   v. deletion of an acidic surface exposed amino acid residue.

In one aspect the invention provides A modified BoNT/A for use in treating facial lines, wherein the modified BoNT/A is administered by intramuscular injection at a plurality of sites of the face of the subject,
wherein the modified BoNT/A is administered by way of a unit dose of 2 Units to 41 Units of modified BoNT/A per site and wherein 1 Unit is an amount of the modified BoNT/A that corresponds to the calculated median lethal dose (LD₅₀) in mice,
wherein the plurality of sites are selected from:
   up to two sites of a corrugator muscle and one site of a procerus muscle for treating glabellar lines,
   up to five sites of a frontalis muscle for treating forehead lines; and
   up to three sites at the external part of a orbicularis oculi muscle for treating lateral canthal lines,
wherein the total dose of modified BoNT/A administered during the treatment is up to 574 Units, and
wherein the modified BoNT/A comprises a BoNT/A light-chain and translocation domain, and a BoNT/B receptor binding domain (H_{c} domain).

In a related aspect the invention provides a method for treating facial lines, the method comprising administering a modified botulinum neurotoxin A (BoNT/A) by intramuscular injection at a plurality of sites of the face of the subject,
wherein the modified BoNT/A is administered by way of a unit dose of 1 Units to 41 Units (2 Units to 41 Units) of modified BoNT/A per site and wherein 1 Unit is an amount of the modified BoNT/A that corresponds to the calculated median lethal dose (LD₅₀) in mice,
wherein the plurality of sites are selected from:
   up to two sites of a corrugator muscle and one site of a procerus muscle for treating glabellar lines,
   up to five sites of a frontalis muscle for treating forehead lines; and
   up to three sites at the external part of a orbicularis oculi muscle for treating lateral canthal lines,
wherein the total dose of modified BoNT/A administered during the treatment is up to 574 Units, and
wherein the modified BoNT/A comprises a modification at one or more amino acid residue(s) selected from: ASN 886, ASN 905, GLN 915, ASN 918, GLU 920, ASN 930, ASN 954, SER 955, GLN 991, GLU 992, GLN 995, ASN 1006, ASN 1025, ASN 1026, ASN 1032, ASN 1043, ASN 1046, ASN 1052, ASP 1058, HIS 1064, ASN 1080, GLU 1081, GLU 1083, ASP 1086, ASN 1188, ASP 1213, GLY 1215, ASN 1216, GLN 1229, ASN 1242, ASN 1243, SER 1274, and THR 1277, wherein the modification is selected from:
   i. substitution of an acidic surface exposed amino acid residue with a basic amino acid residue;
   ii. substitution of an acidic surface exposed amino acid residue with an uncharged amino acid residue;
   iii. substitution of an uncharged surface exposed amino acid residue with a basic amino acid residue;
   iv. insertion of a basic amino acid residue; and
   v. deletion of an acidic surface exposed amino acid residue.

In a related aspect the invention provides a method for treating facial lines, the method comprising administering a modified botulinum neurotoxin A (BoNT/A) by intramuscular injection at a plurality of sites of the face of the subject,
wherein the modified BoNT/A is administered by way of a unit dose of 8.4 pg to 344.4 pg of modified BoNT/A per site,
wherein the plurality of sites are selected from:
   up to two sites of a corrugator muscle and one site of a procerus muscle for treating glabellar lines,
   up to five sites of a frontalis muscle for treating forehead lines; and
   up to three sites at the external part of a orbicularis oculi muscle for treating lateral canthal lines,
wherein the total dose of modified BoNT/A administered during the treatment is up to 574 Units, and
wherein the modified BoNT/A comprises a modification at one or more amino acid residue(s) selected from: ASN 886, ASN 905, GLN 915, ASN 918, GLU 920, ASN 930, ASN 954, SER 955, GLN 991, GLU 992, GLN 995, ASN 1006, ASN 1025, ASN 1026, ASN 1032, ASN 1043, ASN 1046, ASN 1052, ASP 1058, HIS 1064, ASN 1080, GLU 1081, GLU 1083, ASP 1086, ASN 1188, ASP 1213, GLY 1215, ASN 1216, GLN 1229, ASN 1242, ASN 1243, SER 1274, and THR 1277, wherein the modification is selected from:
   i. substitution of an acidic surface exposed amino acid residue with a basic amino acid residue;
   ii. substitution of an acidic surface exposed amino acid residue with an uncharged amino acid residue;
   iii. substitution of an uncharged surface exposed amino acid residue with a basic amino acid residue;
   iv. insertion of a basic amino acid residue; and
   v. deletion of an acidic surface exposed amino acid residue.

In a related aspect, the invention provides a method for treating facial lines, the method comprising administering a modified botulinum neurotoxin A (BoNT/A) by intramuscular injection at a plurality of sites of the face of the subject,
wherein the modified BoNT/A is administered by way of a unit dose of 2 Units to 41 Units of modified BoNT/A per site and wherein 1 Unit is an amount of the modified BoNT/A that corresponds to the calculated median lethal dose (LD₅₀) in mice,
wherein the plurality of sites are selected from:
   up to two sites of a corrugator muscle and one site of a procerus muscle for treating glabellar lines,
   up to five sites of a frontalis muscle for treating forehead lines; and
   up to three sites at the external part of a orbicularis oculi muscle for treating lateral canthal lines,
wherein the total dose of modified BoNT/A administered during the treatment is up to 574 Units, and
wherein the modified BoNT/A comprises a BoNT/A light-chain and translocation domain, and a BoNT/B receptor binding domain (H_{c} domain).

In another related aspect, the invention provides use of a modified botulinum neurotoxin A (BoNT/A) in the manufacture of a medicament for treating facial lines, wherein the modified BoNT/A is administered by intramuscular injection at a plurality of sites of the face of the subject,
wherein the modified BoNT/A is administered by way of a unit dose of 1 Units to 41 Units (2 Units to 41 Units) of modified BoNT/A per site and wherein 1 Unit is an amount of the modified BoNT/A that corresponds to the calculated median lethal dose (LD₅₀) in mice,
wherein the plurality of sites are selected from:
   up to two sites of a corrugator muscle and one site of a procerus muscle for treating glabellar lines,
   up to five sites of a frontalis muscle for treating forehead lines; and
   up to three sites at the external part of a orbicularis oculi muscle for treating lateral canthal lines,
wherein the total dose of modified BoNT/A administered during the treatment is up to 574 Units, and
wherein the modified BoNT/A comprises a modification at one or more amino acid residue(s) selected from: ASN 886, ASN 905, GLN 915, ASN 918, GLU 920, ASN 930, ASN 954, SER 955, GLN 991, GLU 992, GLN 995, ASN 1006, ASN 1025, ASN 1026, ASN 1032, ASN 1043, ASN 1046, ASN 1052, ASP 1058, HIS 1064, ASN 1080, GLU 1081, GLU 1083, ASP 1086, ASN 1188, ASP 1213, GLY 1215, ASN 1216, GLN 1229, ASN 1242, ASN 1243, SER 1274, and THR 1277, wherein the modification is selected from:
   i. substitution of an acidic surface exposed amino acid residue with a basic amino acid residue;
   ii. substitution of an acidic surface exposed amino acid residue with an uncharged amino acid residue;
   iii. substitution of an uncharged surface exposed amino acid residue with a basic amino acid residue;
   iv. insertion of a basic amino acid residue; and
   v. deletion of an acidic surface exposed amino acid residue.

In another related aspect, the invention provides use of a modified botulinum neurotoxin A (BoNT/A) in the manufacture of a medicament for treating facial lines, wherein the modified BoNT/A is administered by intramuscular injection at a plurality of sites of the face of the subject,
wherein the modified BoNT/A is administered by way of a unit dose of 8.4 pg to 344.4 pg of modified BoNT/A per site,
wherein the plurality of sites are selected from:
   up to two sites of a corrugator muscle and one site of a procerus muscle for treating glabellar lines,
   up to five sites of a frontalis muscle for treating forehead lines; and
   up to three sites at the external part of a orbicularis oculi muscle for treating lateral canthal lines,
wherein the total dose of modified BoNT/A administered during the treatment is up to 574 Units, and
wherein the modified BoNT/A comprises a modification at one or more amino acid residue(s) selected from: ASN 886, ASN 905, GLN 915, ASN 918, GLU 920, ASN 930, ASN 954, SER 955, GLN 991, GLU 992, GLN 995, ASN 1006, ASN 1025, ASN 1026, ASN 1032, ASN 1043, ASN 1046, ASN 1052, ASP 1058, HIS 1064, ASN 1080, GLU 1081, GLU 1083, ASP 1086, ASN 1188, ASP 1213, GLY 1215, ASN 1216, GLN 1229, ASN 1242, ASN 1243, SER 1274, and THR 1277, wherein the modification is selected from:
   i. substitution of an acidic surface exposed amino acid residue with a basic amino acid residue;
   ii. substitution of an acidic surface exposed amino acid residue with an uncharged amino acid residue;
   iii. substitution of an uncharged surface exposed amino acid residue with a basic amino acid residue;
   iv. insertion of a basic amino acid residue; and
   v. deletion of an acidic surface exposed amino acid residue.

In another related aspect, the invention provides use of a modified botulinum neurotoxin A (BoNT/A) in the manufacture of a medicament for treating facial lines, wherein the modified BoNT/A is administered by intramuscular injection at a plurality of sites of the face of the subject,
wherein the modified BoNT/A is administered by way of a unit dose of 2 Units to 41 Units of modified BoNT/A per site and wherein 1 Unit is an amount of the modified BoNT/A that corresponds to the calculated median lethal dose (LD₅₀) in mice,
wherein the plurality of sites are selected from:
   up to two sites of a corrugator muscle and one site of a procerus muscle for treating glabellar lines,
   up to five sites of a frontalis muscle for treating forehead lines; and
   up to three sites at the external part of a orbicularis oculi muscle for treating lateral canthal lines,
wherein the total dose of modified BoNT/A administered during the treatment is up to 574 Units, and
wherein the modified BoNT/A comprises a BoNT/A light-chain and translocation domain, and a BoNT/B receptor binding domain (H_{c} domain).

In one aspect, the invention provides a modified BoNT/A for use in treating facial lines, wherein the modified BoNT/A is administered by intramuscular injection at a plurality of sites of the face of the subject,
wherein the modified BoNT/A is administered by way of a unit dose of 0.5 Units to 73 Units of modified BoNT/A per site and wherein 1 Unit is an amount of the modified BoNT/A that corresponds to the calculated median lethal dose (LD₅₀) in mice,
wherein the plurality of sites are selected from:
   up to two sites of a corrugator muscle and one site of a procerus muscle for treating glabellar lines,
   up to five sites of a frontalis muscle for treating forehead lines; and
   up to three sites at the external part of an orbicularis oculi muscle for treating lateral canthal lines,
wherein the total dose of modified BoNT/A administered during the treatment is up to 1019 Units, and
wherein the modified BoNT/A comprises a BoNT/A light-chain and translocation domain, and a BoNT/B receptor binding domain (H_{c} domain).

In another aspect, the invention provides a modified BoNT/A for use in treating facial lines, wherein the modified BoNT/A is administered by intramuscular injection at a plurality of sites of the face of the subject,
wherein the modified BoNT/A is administered by way of a unit dose of 12 pg to 1754 pg of modified BoNT/A per site,
wherein the plurality of sites are selected from:
   up to two sites of a corrugator muscle and one site of a procerus muscle for treating glabellar lines,
   up to five sites of a frontalis muscle for treating forehead lines; and
   up to three sites at the external part of an orbicularis oculi muscle for treating lateral canthal lines,
wherein the total dose of modified BoNT/A administered during the treatment is up to 24,500 pg, and
wherein the modified BoNT/A comprises a BoNT/A light-chain and translocation domain, and a BoNT/B receptor binding domain (H_{c} domain).

In a related aspect, the invention provides a method for treating facial lines, the method comprising administering a modified botulinum neurotoxin A (BoNT/A) by intramuscular injection at a plurality of sites of the face of the subject,
wherein the modified BoNT/A is administered by way of a unit dose of 0.5 Units to 73 Units of modified BoNT/A per site and wherein 1 Unit is an amount of the modified BoNT/A that corresponds to the calculated median lethal dose (LD₅₀) in mice,
wherein the plurality of sites are selected from:
   up to two sites of a corrugator muscle and one site of a procerus muscle for treating glabellar lines,
   up to five sites of a frontalis muscle for treating forehead lines; and
   up to three sites at the external part of an orbicularis oculi muscle for treating lateral canthal lines,
wherein the total dose of modified BoNT/A administered during the treatment is up to 1019 Units, and
wherein the modified BoNT/A comprises a BoNT/A light-chain and translocation domain, and a BoNT/B receptor binding domain (H_{c} domain).

In a related aspect, the invention provides a method for treating facial lines, the method comprising administering a modified botulinum neurotoxin A (BoNT/A) by intramuscular injection at a plurality of sites of the face of the subject,
wherein the modified BoNT/A is administered by way of a unit dose of 12 pg to 1754 pg of modified BoNT/A per site,
wherein the plurality of sites are selected from:
   up to two sites of a corrugator muscle and one site of a procerus muscle for treating glabellar lines,
   up to five sites of a frontalis muscle for treating forehead lines; and
   up to three sites at the external part of an orbicularis oculi muscle for treating lateral canthal lines,
wherein the total dose of modified BoNT/A administered during the treatment is up to 24,500 pg, and
wherein the modified BoNT/A comprises a BoNT/A light-chain and translocation domain, and a BoNT/B receptor binding domain (H_{c} domain).

In another related aspect, the invention provides use of a modified botulinum neurotoxin A (BoNT/A) in the manufacture of a medicament for treating facial lines, wherein the modified BoNT/A is administered by intramuscular injection at a plurality of sites of the face of the subject,
wherein the modified BoNT/A is administered by way of a unit dose of 0.5 Units to 73 Units of modified BoNT/A per site and wherein 1 Unit is an amount of the modified BoNT/A that corresponds to the calculated median lethal dose (LD₅₀) in mice,
wherein the plurality of sites are selected from:
   up to two sites of a corrugator muscle and one site of a procerus muscle for treating glabellar lines,
   up to five sites of a frontalis muscle for treating forehead lines; and
   up to three sites at the external part of an orbicularis oculi muscle for treating lateral canthal lines,
wherein the total dose of modified BoNT/A administered during the treatment is up to 1019 Units, and
wherein the modified BoNT/A comprises a BoNT/A light-chain and translocation domain, and a BoNT/B receptor binding domain (H_{c} domain).

In another related aspect, the invention provides use of a modified botulinum neurotoxin A (BoNT/A) in the manufacture of a medicament for treating facial lines, wherein the modified BoNT/A is administered by intramuscular injection at a plurality of sites of the face of the subject,
wherein the modified BoNT/A is administered by way of a unit dose of 12 pg to 1754 pg of modified BoNT/A per site,
wherein the plurality of sites are selected from:
   up to two sites of a corrugator muscle and one site of a procerus muscle for treating glabellar lines,
   up to five sites of a frontalis muscle for treating forehead lines; and
   up to three sites at the external part of an orbicularis oculi muscle for treating lateral canthal lines,
wherein the total dose of modified BoNT/A administered during the treatment is up to 24,500 pg, and
wherein the modified BoNT/A comprises a BoNT/A light-chain and translocation domain, and a BoNT/B receptor binding domain (H_{c} domain).

Reference to “muscle” means an affected muscle that contributes (e.g. via excess tightness, tension, or tone) to an underlying aesthetic condition that the present invention addresses.

A first group of muscles is defined by a procerus muscle, a second group of muscles is defined by a corrugator muscle, a third group of muscles is defined by an orbicularis oculi muscle, and a fourth group of muscles is defined by a frontalis muscle.

The plurality of sites may be on the same muscle group (eg. right and left corrugator muscle, right and left orbicularis oculi muscle). Similarly, the plurality of sites may be on the same muscle (eg. right corrugator muscle or left corrugator muscle; or right orbicularis oculi muscle or left orbicularis oculi muscle). Similarly, the plurality of sites may be on a combination of said muscle and muscle groups. Thus, a personalised treatment regimen may be provided.

A modified BoNT/A for use in the present invention may be either a modified BoNT/A comprising a modification at one or more amino acid residue(s) selected from: ASN 886, ASN 905, GLN 915, ASN 918, GLU 920, ASN 930, ASN 954, SER 955, GLN 991, GLU 992, GLN 995, ASN 1006, ASN 1025, ASN 1026, ASN 1032, ASN 1043, ASN 1046, ASN 1052, ASP 1058, HIS 1064, ASN 1080, GLU 1081, GLU 1083, ASP 1086, ASN 1188, ASP 1213, GLY 1215, ASN 1216, GLN 1229, ASN 1242, ASN 1243, SER 1274, and THR 1277, wherein the modification is selected from: substitution of an acidic surface exposed amino acid residue with a basic amino acid residue; substitution of an acidic surface exposed amino acid residue with an uncharged amino acid residue; substitution of an uncharged surface exposed amino acid residue with a basic amino acid residue; insertion of a basic amino acid residue; and deletion of an acidic surface exposed amino acid residue or a modified BoNT/A comprising a BoNT/A light-chain and translocation domain, and a BoNT/B receptor binding domain (H_{c} domain).

Preferably, a modified BoNT/A for use in the present invention is a modified BoNT/A comprising a modification at one or more amino acid residue(s) selected from: ASN 886, ASN 905, GLN 915, ASN 918, GLU 920, ASN 930, ASN 954, SER 955, GLN 991, GLU 992, GLN 995, ASN 1006, ASN 1025, ASN 1026, ASN 1032, ASN 1043, ASN 1046, ASN 1052, ASP 1058, HIS 1064, ASN 1080, GLU 1081, GLU 1083, ASP 1086, ASN 1188, ASP 1213, GLY 1215, ASN 1216, GLN 1229, ASN 1242, ASN 1243, SER 1274, and THR 1277, wherein the modification is selected from: substitution of an acidic surface exposed amino acid residue with a basic amino acid residue; substitution of an acidic surface exposed amino acid residue with an uncharged amino acid residue; substitution of an uncharged surface exposed amino acid residue with a basic amino acid residue; insertion of a basic amino acid residue; and deletion of an acidic surface exposed amino acid residue.

Potency of a modified BoNT/A for use according to the invention is preferably determined by a mouse LD₅₀ assay according to standard techniques. In said assay, 1 Unit is defined as an amount of the modified BoNT/A that corresponds to the calculated median lethal dose (LD₅₀) in mice. Preferably, the calculated median lethal intraperitoneal dose in mice. An amount of a modified BoNT/A that corresponds to 1 Unit in said assay may be at least 1 pg, 2 pg, 3 pg, 4 pg, 5 pg, 6 pg, 7 pg, 8 pg or 9 pg. An amount of a modified BoNT/A that corresponds to 1 Unit in said assay may be ≤45 pg, ≤40 pg, ≤30 pg, ≤25 pg, ≤20 pg, ≤19 pg, ≤18 pg, ≤17 pg, ≤16 pg, ≤15 pg, ≤14 pg, ≤13 pg, ≤12 pg, ≤11 pg, ≤10 pg, ≤9 pg, ≤8 pg, ≤7 pg or ≤6 pg.

Where a modified BoNT/A for use in the invention is a modified BoNT/A comprising a modification at one or more amino acid residue(s) selected from: ASN 886, ASN 905, GLN 915, ASN 918, GLU 920, ASN 930, ASN 954, SER 955, GLN 991, GLU 992, GLN 995, ASN 1006, ASN 1025, ASN 1026, ASN 1032, ASN 1043, ASN 1046, ASN 1052, ASP 1058, HIS 1064, ASN 1080, GLU 1081, GLU 1083, ASP 1086, ASN 1188, ASP 1213, GLY 1215, ASN 1216, GLN 1229, ASN 1242, ASN 1243, SER 1274, and THR 1277, wherein the modification is selected from: substitution of an acidic surface exposed amino acid residue with a basic amino acid residue; substitution of an acidic surface exposed amino acid residue with an uncharged amino acid residue; substitution of an uncharged surface exposed amino acid residue with a basic amino acid residue; insertion of a basic amino acid residue; and deletion of an acidic surface exposed amino acid residue, an amount of a modified BoNT/A that corresponds to 1 Unit in said assay may be 1-15 pg, such as 5-10 pg. Preferably, an amount of a modified BoNT/A that corresponds to 1 Unit in said assay may be 8-9 pg, more preferably 8.4 pg.

Where a modified BoNT/A for use in the invention is modified BoNT/A comprising a BoNT/A light-chain and translocation domain, and a BoNT/B receptor binding domain (H_{c} domain), an amount of a modified BoNT/A that corresponds to 1 Unit in said assay may be 15-35 pg, such as 20-30 pg. Preferably, an amount of a modified BoNT/A that corresponds to 1 Unit in said assay may be 23-25 pg, more preferably 24.0 pg.

The term “up to” when used in reference to a value (e.g. up to 574 Units) means up to and including the value recited. Thus, as an example, reference to administering “up to 574 Units” of modified BoNT/A encompasses administration of 574 Units of modified BoNT/A as well as administration of less than 574 Units of modified BoNT/A.

A dose of modified BoNT/A is administered by intramuscular injection at a plurality of sites. A single unit dose is administered per site. The term “a single unit dose is administered” means substantially all of a single unit dose is administered. Preferably at most only a residual amount (e.g. up to 1%, 0.1% or 0.01%) of the unit dose may remain in a vial in which the modified BoNT/A has been reconstituted. However, preferably all of a single unit dose is administered. Depending on the muscle group, a single unit dose is typically administered to a muscle selected from the first group (described herein), a 2x unit is typically administered to a muscle selected from the second group (described herein), a 3x unit is typically administered to a muscle selected from the third group (described herein), and a 5x unit is typically administered to a muscle selected from the fourth group (described herein).

The unit dose can be expressed in terms of Units of modified BoNT/A.

The unit dose may be 2 Units to 41 Units of modified BoNT/A. An upper limit of the unit dose range may be 40, 35, 30, 25, 20, 15 or 10. Units of modified BoNT/A, preferably the upper limit is 35 Units. A lower limit of the unit dose range may be 5, 10, 15, 20, 25, 30 or 35. Preferably, the unit dose of modified BoNT/A is 12 Units to 35 Units, 12 Units to 24 Units, more preferably 12 to 18 Units.

Alternatively or additionally, a unit dose may be expressed in terms of an amount of modified BoNT/A. Thus, in one aspect the invention provides a modified botulinum neurotoxin A (BoNT/A) for use in treating facial lines, wherein the modified BoNT/A is administered by intramuscular injection at a plurality of sites of the face of the subject,
wherein the modified BoNT/A is administered by way of a unit dose of 18 pg to 350 pg of modified BoNT/A per site,
wherein the plurality of sites are selected from:
   up to two sites of a corrugator muscle and one site of a procerus muscle for treating glabellar lines,
   up to five sites of a frontalis muscle for treating forehead lines; and
   up to three sites at the external part of a orbicularis oculi muscle for treating lateral canthal lines,
wherein the total dose of modified BoNT/A administered during the treatment is up to 4850 pg, and
wherein the modified BoNT/A comprises a modification at one or more amino acid residue(s) selected from: ASN 886, ASN 905, GLN 915, ASN 918, GLU 920, ASN 930, ASN 954, SER 955, GLN 991, GLU 992, GLN 995, ASN 1006, ASN 1025, ASN 1026, ASN 1032, ASN 1043, ASN 1046, ASN 1052, ASP 1058, HIS 1064, ASN 1080, GLU 1081, GLU 1083, ASP 1086, ASN 1188, ASP 1213, GLY 1215, ASN 1216, GLN 1229, ASN 1242, ASN 1243, SER 1274, and THR 1277, wherein the modification is selected from:
   i. substitution of an acidic surface exposed amino acid residue with a basic amino acid residue;
   ii. substitution of an acidic surface exposed amino acid residue with an uncharged amino acid residue;
   iii. substitution of an uncharged surface exposed amino acid residue with a basic amino acid residue;
   iv. insertion of a basic amino acid residue; and
   v. deletion of an acidic surface exposed amino acid residue.

In another aspect, the invention provides a modified BoNT/A for use in treating facial lines, wherein the modified BoNT/A is administered by intramuscular injection at a plurality of sites of the face of the subject,
wherein the modified BoNT/A is administered by way of a unit dose of 18 pg to 350 pg of modified BoNT/A per site,
wherein the plurality of sites are selected from:
   up to two sites of a corrugator muscle and one site of a procerus muscle for treating glabellar lines,
   up to five sites of a frontalis muscle for treating forehead lines; and
   up to three sites at the external part of a orbicularis oculi muscle for treating lateral canthal lines,
wherein the total dose of modified BoNT/A administered during the treatment is up to 4850 pg, and
wherein the modified BoNT/A comprises a BoNT/A light-chain and translocation domain, and a BoNT/B receptor binding domain (H_{c} domain).

Corresponding uses (in the manufacture of a medicament) and methods of treatment are also provided.

The unit dose may be 18 pg to 350 pg of modified BoNT/A. An upper limit of the unit dose range may be 325, 300, 275, 250, 225, 200, 175, 150, 125, 100 or 50 pg of modified BoNT/A, preferably the upper limit is 300 pg. A lower limit of the unit dose range may be 20, 25, 30, 35, 40, 45, 50, 60, 65, 70, 75, 80, 85, 90, 100, 125, 150, 175, 200, 250 or 300 pg of modified BoNT/A, preferably the lower limit is 20 pg. The unit dose of modified BoNT/A is selected from: 18 pg to 350 pg, 20 pg to 300 pg, most preferably 100 pg to 150 pg. Preferably, the unit dose of modified BoNT/A is 20 pg to 300 pg of modified BoNT/A, e.g. 50 pg to 250 pg.

Most preferably a unit dose of modified BoNT/A is 80 to 180 pg, such as 100 pg to 150 pg.

The unit dose of modified BoNT/A may be 2 Units to 41 Units. An upper limit of the unit dose range may be 35, 30, 25, 20, 15, 10, or 5 Units of modified BoNT/A, preferably the upper limit is 35 Units. A lower limit of the unit dose range may be 0.5, 0.6, 0.7, 0.8, 0.9, 1, 1.1., 1.2, 1.3, 1.4, 1.5, 1.6, 1.7, 1.8, 1.9 or 2 Units of modified BoNT/A, preferably the lower limit is 2 Units. Preferably, the unit dose of modified BoNT/A is 2 Units to 35 Units of modified BoNT/A, e.g. 15 Units to 25 Units.

The unit dose of modified BoNT/A is selected from: 2 Units to 35 Units, 6 to 35 Units, 12 Units to 35 Units, 12 Units to 24 Units, most preferably 12 Units to 18 Units.

Most preferably a unit dose of modified BoNT/A is 5 to 25 Units, such as 12 Units to 18 Units.

The unit dose may be 8.4 pg to 350 pg of modified BoNT/A. An upper limit of the unit dose range may be 325, 300, 275, 250, 225, 200, 175, 150, 125, 100 or 50 pg of modified BoNT/A, preferably the upper limit is 300 pg. A lower limit of the unit dose range may be 5, 10, 20, 25, 30, 35, 40, 45, 50, 60, 65, 70, 75, 80, 85, 90, 100, 125, 150, 175, 200, 250 or 300 pg of modified BoNT/A, preferably the lower limit is 10 pg. Preferably, the unit dose of modified BoNT/A is 10 pg to 300 pg of modified BoNT/A, more preferably 10 pg to 200 pg, e.g. 50 pg to 250 pg.

The unit dose of modified BoNT/A is selected from: 8.4 pg to 350 pg, 10 pg to 300 pg, most preferably 60 pg to 120 pg.

Most preferably a unit dose of modified BoNT/A is 80 to 180 pg, such as 60 pg to 120 pg.

The unit dose of modified BoNT/A may be 1 Units to 41 Units. An upper limit of the unit dose range may be 35, 30, 25, 20, 15, 10, or 5 Units of modified BoNT/A, preferably the upper limit is 35 Units. A lower limit of the unit dose range may be 0.1, 0.2, 0.3, 0.4, 0.5, 0.6, 0.7, 0.8, 0.9, or 1 Unit of modified BoNT/A, preferably the lower limit is 1 Unit. Preferably, the unit dose of modified BoNT/A is 1 to 35 Units of modified BoNT/A, e.g. 10 to 20 Units.

The unit dose of modified BoNT/A is selected from: 1 Units to 35 Units, 3.5 to 35 Units, 7 Units to 35 Units, 7 Units to 24 Units, most preferably 7 Units to 14 Units.

Most preferably a unit dose of modified BoNT/A is 5 Units to 20 Units, such as 7 Units to 14 Units.

The unit dose may be 0.5 Units to 73 Units of modified BoNT/A. An upper limit of the unit dose range may be 70, 65, 60, 55, 50, 45, 40, 35, 30, 25, 20, 15, 10 or 5 Units of modified BoNT/A, preferably the upper limit is 62 Units. A lower limit of the unit dose range may be 0.5, 0.6, 0.7, 0.8, 0.9, 1, 1.5, 2, 2.5, 3, 3.5, 4, 4.5, 5, 5.5, 6, 6.5, 7, 7.5, 8, 8.5 or 9 Units of modified BoNT/A, preferably the lower limit is 0.8 Units. Preferably, the unit dose of modified BoNT/A is 0.8 Units to 62 Units of modified BoNT/A, e.g. 20 to 40 Units.

Most preferably a unit dose of modified BoNT/A is 5 Units to 25 Units, such as 10 Units to 21 Units.

The unit dose may be 12 pg to 1754 pg of modified BoNT/A. An upper limit of the unit dose range may be 1700, 1600, 1500, 1400, 1300, 1200, 1100, 1000, 800, 600, 400, 200, 100 or 50 pg of modified BoNT/A, preferably the upper limit is 1000 pg. A lower limit of the unit dose range may be 5, 10, 20, 25, 30, 35, 40, 45, 50, 60, 65, 70, 75, 80, 85, 90, 100, 125, 150, 175, 200, 250 or 300 pg of modified BoNT/A, preferably the lower limit is 20 pg. Preferably, the unit dose of modified BoNT/A is 20 pg to 1500 pg, more preferably 20 pg to 1000 pg of modified BoNT/A, e.g. 500 pg to 750 pg.

Most preferably a unit dose of modified BoNT/A is 200 to 750 pg, such as 250 pg to 500 pg.

These unit dosages may be particular relevant where the modified BoNT/A comprises a BoNT/A light-chain and translocation domain, and a BoNT/B receptor binding domain (H_{c} domain).

A unit dose of modified BoNT/A may also be expressed in both Units and amounts (pg) simultaneously.

When treating facial lines modified BoNT/A may be administered to a plurality of sites selected from: up to two sites of a corrugator muscle and one site of a procerus muscle for treating glabellar lines, up to five sites of a frontalis muscle for treating forehead lines; and up to three sites at the external part of a orbicularis oculi muscle for treating lateral canthal lines.

A total dose administered when carrying out the treatment regimen of the present invention may be up to 574 Units. In other words, the total amount of modified BoNT/A administered at a given treatment session may be up to 574 Units. The total dose may be up to 570, 560, 550, 540, 530, 520, 510, 500, 490, 480, 470, 460, 450 or 440 Units. Preferably, the total dose may be up to 288 Units of modified BoNT/A.

A total dose administered when carrying out the treatment regimen of the present invention may be up to 4850, 4500, 4000, 3550, 3000, 2750, 2500 pg, preferably up to 2400 pg.

A total dose administered when carrying out the treatment regimen for glabellar lines may be up to 1500, 1400, 1300, 1200, 1100, 1000, 800, 600, 400, 200, 100, or 50, preferably up to 1000 pg.

A total dose administered when carrying out the treatment regimen for forehead lines may be up to 1500, 1400, 1300, 1200, 1100, 1000, 800, 600, 400, 200, 100, or 50, preferably up to 1000 pg.

A total dose administered when carrying out the treatment regimen for lateral canthal lines may be up to 1800, 1700, 1600, 1500, 1400, 1300, 1200, 1100, 1000, 800, 600, 400, 200, 80, 60, 40 or 20, preferably up to 1200 pg.

A total dose administered when carrying out the treatment regimen of the present invention may be up to 1019 Units. In other words, the total amount of modified BoNT/A administered at a given treatment session may be up to 1019 Units. The total dose may be up to 1000, 950, 900, 850, 800, 750, 700, 650, 600, 550, 500, 450, 400 or 400 Units. Preferably, the total dose may be up to 333 Units of modified BoNT/A.

A total dose administered when carrying out the treatment regimen of the present invention may be up to 24,500, 24,000, 23,500, 23,000, 22,500, 22,000 pg, preferably up to 8,000 pg. These total dosages may be particular relevant where the modified BoNT/A comprises a BoNT/A light-chain and translocation domain, and a BoNT/B receptor binding domain (H_{c} domain).

A total dose administered when carrying out the treatment regimen for glabellar lines may be up to 5000, 4500, 4000, 3500, 3000, 2500, 2000, 1500, 1000, 500, 400, 300, 200, 100, or 50, preferably up to 5000 pg.

A total dose administered when carrying out the treatment regimen for forehead lines may be up to 5000, 4500, 4000, 3500, 3000, 2500, 2000, 1500, 1000, 500, 400, 300, 200, 100, or 50, preferably up to 5000 pg.

A total dose administered when carrying out the treatment regimen for lateral canthal lines may be up to 6000, 5500, 4500, 4000, 3500, 3000, 2500, 2000, 1000, 500, 400, 300, 200, 100, or 50, preferably up to 6000 pg.

These total dosages may be particular relevant where the modified BoNT/A comprises a BoNT/A light-chain and translocation domain, and a BoNT/B receptor binding domain (H_{c} domain).

The skilled person will take into consideration when a subject has recently had (or is subsequently having) additional treatment with a clostridial neurotoxin (e.g. BoNT), e.g. as part of a cosmetic treatment or treatment for a different indication. Using techniques routine in the art, the skilled person will adapt the present treatment regimen accordingly.

Administration to the plurality of muscles in accordance with the present invention preferably occurs in the same treatment session.

Treatment may be repeated at an appropriate time period following administration of modified BoNT/A. Given that the duration of action is approximately twice that of unmodified BoNT/A (e.g. Dysport®) there are suitably longer periods between subsequent administrations than when a subject is treated with unmodified BoNT/A (e.g. Dysport®). A subject may be re-administered a modified BoNT/A in accordance with the present invention at least 18, 20, 25 or 30 weeks following a previous administration. For example, a subject may be re-administered a modified BoNT/A in accordance with the present invention at least 18-45 weeks, preferably 20-35 weeks following a previous administration.

A “subject” as used herein may be a mammal, such as a human or other mammal. Preferably “subject” means a human subject.

The term “treat” or “treating” as used herein encompasses prophylactic treatment (e.g. to prevent onset of a disorder) as well as corrective treatment (treatment of a subject already suffering from a disorder). Preferably “treat” or “treating” as used herein means corrective treatment. The term “treat” or “treating” as used herein refers to the disorder and/or a symptom thereof.

Suitable modified BoNT/A polypeptides (and nucleotide sequences encoding the same, where present) are described in WO 2015/004461 A1 and WO 2017/191315, both of which are incorporated herein by reference in their entirety.

BoNT/A is one example of a clostridial neurotoxin produced by bacteria in the genus Clostridia. Other examples of such clostridial neurotoxins include those produced by C. tetani (TeNT) and by C. botulinum (BoNT) serotypes B-G, as well as those produced by C. baratii and C. butyricum. Said neurotoxins are highly potent and specific and can poison neurons and other cells to which they are delivered. Among the clostridial toxins are some of the most potent toxins known. By way of example, botulinum neurotoxins have median lethal dose (LD₅₀) values for mice ranging from 0.5 to 5 ng/kg, depending on the serotype. Both tetanus and botulinum toxins act by inhibiting the function of affected neurons, specifically the release of neurotransmitters. While botulinum toxin acts at the neuromuscular junction and inhibits cholinergic transmission in the peripheral nervous system, tetanus toxin acts in the central nervous system.

In nature, clostridial neurotoxins (including BoNT/A) are synthesised as a single-chain polypeptide that is modified post-translationally by a proteolytic cleavage event to form two polypeptide chains joined together by a disulphide bond. Cleavage occurs at a specific cleavage site, often referred to as the activation site, that is located between the cysteine residues that provide the inter-chain disulphide bond. It is this di-chain form that is the active form of the toxin. The two chains are termed the heavy chain (H-chain), which has a molecular mass of approximately 100 kDa, and the light chain (L-chain), which has a molecular mass of approximately 50 kDa. The H-chain comprises an N-terminal translocation component (H_{N} domain) and a C-terminal targeting component (H_{C} domain). The cleavage site is located between the L-chain and the translocation domain components. Following binding of the H_{C} domain to its target neuron and internalisation of the bound toxin into the cell via an endosome, the H_{N} domain translocates the L-chain across the endosomal membrane and into the cytosol, and the L-chain provides a protease function (also known as a non-cytotoxic protease).

Non-cytotoxic proteases act by proteolytically cleaving intracellular transport proteins known as SNARE proteins (e.g. SNAP-25, VAMP, or Syntaxin) - see Gerald K (2002) “Cell and Molecular Biology” (4th edition) John Wiley & Sons, Inc. The acronym SNARE derives from the term Soluble NSF Attachment Receptor, where NSF means N-ethylmaleimide-Sensitive Factor. SNARE proteins are integral to intracellular vesicle fusion, and thus to secretion of molecules via vesicle transport from a cell. The protease function is a zinc-dependent endopeptidase activity and exhibits a high substrate specificity for SNARE proteins. Accordingly, once delivered to a desired target cell, the non-cytotoxic protease is capable of inhibiting cellular secretion from the target cell. The L-chain proteases of clostridial toxins are non-cytotoxic proteases that cleave SNARE proteins.

The term “H_{C} domain” as used herein means a functionally distinct region of a neurotoxin heavy chain with a molecular weight of approximately 50 kDa that enables the binding of the neurotoxin to a receptor located on the surface of the target cell. The H_{C} domain consists of two structurally distinct subdomains, the “H_{CN} subdomain” (N-terminal part of the H_{C} domain) and the “H_{CC} subdomain” (C-terminal part of the H_{C} domain), each of which has a molecular weight of approximately 25 kDa.

The term “LH_{N} domain” as used herein means a neurotoxin that is devoid of the H_{C} domain and consists of an endopeptidase domain (“L” or “light chain”) and the domain responsible for translocation of the endopeptidase into the cytoplasm (H_{N} domain of the heavy chain).

As discussed above, clostridial toxins are formed from two polypeptide chains, the heavy chain (H-chain), which has a molecular mass of approximately 100 kDa, and the light chain (L-chain), which has a molecular mass of approximately 50 kDa. The H-chain comprises a C-terminal targeting component (receptor binding domain or H_{C} domain) and an N-terminal translocation component (H_{N} domain).

Examples of light chain reference sequences include:
Botulinum type A neurotoxin: amino acid residues 1-448
Botulinum type B neurotoxin: amino acid residues 1-440

The above-identified reference sequences should be considered a guide, as slight variations may occur according to sub-serotypes. By way of example, US 2007/0166332 (hereby incorporated by reference in its entirety) cites slightly different clostridial sequences:
Botulinum type A neurotoxin: amino acid residues M1-K448
Botulinum type B neurotoxin: amino acid residues M1-K441

A preferred modified BoNT/A is one that comprises a modification at one or more amino acid residue(s) selected from: ASN 886, ASN 905, GLN 915, ASN 918, GLU 920, ASN 930, ASN 954, SER 955, GLN 991, GLU 992, GLN 995, ASN 1006, ASN 1025, ASN 1026, ASN 1032, ASN 1043, ASN 1046, ASN 1052, ASP 1058, HIS 1064, ASN 1080, GLU 1081, GLU 1083, ASP 1086, ASN 1188, ASP 1213, GLY 1215, ASN 1216, GLN 1229, ASN 1242, ASN 1243, SER 1274, and THR 1277. Such modified BoNT/A demonstrates a reduction in, or absence of, side effects compared to the use of known BoNT/A. The increased tissue retention properties of the modified BoNT/A of the invention also provides increased potency and/or duration of action and can allow for reduced dosages to be used compared to known clostridial toxin therapeutics (or increased dosages without any additional adverse effects), thus providing further advantages.

The modification may be a modification when compared to unmodified BoNT/A shown as SEQ ID NO: 2, wherein the amino acid residue numbering is determined by alignment with SEQ ID NO: 2. As the presence of a methionine residue at position 1 of SEQ ID NO: 2 (as well as the SEQ ID NOs corresponding to modified BoNT/A polypeptides described herein) is optional, the skilled person will take the presence/absence of the methionine residue into account when determining amino acid residue numbering. For example, where SEQ ID NO: 2 includes a methionine, the position numbering will be as defined above (e.g. ASN 886 will be ASN 886 of SEQ ID NO: 2). Alternatively, where the methionine is absent from SEQ ID NO: 2 the amino acid residue numbering should be modified by -1 (e.g. ASN 886 will be ASN 885 of SEQ ID NO: 2). Similar considerations apply when the methionine at position 1 of the other polypeptide sequences described herein is present/absent, and the skilled person will readily determine the correct amino acid residue numbering using techniques routine in the art.

The amino acid residue(s) indicated for modification are surface exposed amino acid residue(s).

A modified BoNT/A may comprise a modification at one or more amino acid residue(s) selected from: ASN 886, ASN 930, ASN 954, SER 955, GLN 991, ASN 1025, ASN 1026, ASN 1052, ASN 1188, ASP 1213, GLY 1215, ASN 1216, GLN 1229, ASN 1242, ASN 1243, SER 1274 and THR 1277. The modified BoNT/A may be encoded by a nucleic acid sequence having at least 70% sequence identity to a nucleic acid sequence selected from SEQ ID NOs: 3, 5, 7, and 9. For example, a nucleic acid sequence having at least 80%, 90%, 95% or 99.9% sequence identity to a nucleic acid sequence selected from SEQ ID NOs: 3, 5, 7, and 9. Preferably, a modified BoNT/A for use in the invention may be encoded by a nucleic acid comprising (or consisting of) SEQ ID NO: 3, 5, 7 or 9. The modified BoNT/A may comprise a polypeptide sequence having at least 70% sequence identity to a polypeptide sequence selected from SEQ ID NOs: 4, 6, 8, and 10. For example, a polypeptide sequence having at least 80%, 90%, 95% or 99.9% sequence identity to a polypeptide sequence selected from SEQ ID NOs: 4, 6, 8, and 10. Preferably, a modified BoNT/A for use in the invention may comprise (more preferably consist of) a polypeptide sequence selected from SEQ ID NOs: 4, 6, 8, and 10.

The term “one or more amino acid residue(s)” when used in the context of modified BoNT/A preferably means at least 2, 3, 4, 5, 6 or 7 of the indicated amino acid residue(s). Thus, a modified BoNT/A may comprise at least 2, 3, 4, 5, 6 or 7 (preferably 7) modifications at the indicated amino acid residue(s). A modified BoNT/A may comprise 1-30, 3-20, or 5-10 amino acid modifications. More preferably, the term “one or more amino acid residue(s)” when used in the context of modified BoNT/A means all of the indicated amino acid residue(s).

Preferably, beyond the one or more amino acid modification(s) at the indicated amino acid residue(s), the modified BoNT/A does not contain any further amino acid modifications when compared to SEQ ID NO: 2.

Most preferably, a modified BoNT/A comprises (more preferably consists of) a modification at one or more amino acid residue(s) selected from: ASN 886, ASN 930, SER 955, GLN 991, ASN 1026, ASN 1052, and GLN 1229. The modified BoNT/A may be encoded by a nucleic acid sequence having at least 70% sequence identity to SEQ ID NO: 3. For example, a nucleic acid sequence having at least 80%, 90%, 95% or 99.9% sequence identity to SEQ ID NO: 3. Preferably, a modified BoNT/A for use in the invention may be encoded by a nucleic acid comprising (or consisting of) SEQ ID NO: 3. The modified BoNT/A may comprise a polypeptide sequence having at least 70% sequence identity to SEQ ID NO: 4. For example, a polypeptide sequence having at least 80%, 90%, 95% or 99.9% sequence identity to SEQ ID NO: 4. Preferably, a modified BoNT/A for use in the invention may comprise (more preferably consist of) SEQ ID NO: 4.

The modification may be selected from:
i. substitution of an acidic surface exposed amino acid residue with a basic amino acid residue;
ii. substitution of an acidic surface exposed amino acid residue with an uncharged amino acid residue;
iii. substitution of an uncharged surface exposed amino acid residue with a basic amino acid residue;
iv. insertion of a basic amino acid residue; and
v. deletion of an acidic surface exposed amino acid residue.

A modification as indicated above results in a modified BoNT/A that has an increased positive surface charge and increased isoelectric point when compared to the corresponding unmodified BoNT/A.

The isoelectric point (pl) is a specific property of a given protein. As is well known in the art, proteins are made from a specific sequence of amino acids (also referred to when in a protein as amino acid residues). Each amino acid of the standard set of twenty has a different side chain (or R group), meaning that each amino acid residue in a protein displays different chemical properties such as charge and hydrophobicity. These properties may be influenced by the surrounding chemical environment, such as the temperature and pH. The overall chemical characteristics of a protein will depend on the sum of these various factors.

Certain amino acid residues (detailed below) possess ionisable side chains that may display an electric charge depending on the surrounding pH. Whether such a side chain is charged or not at a given pH depends on the pKa of the relevant ionisable moiety, wherein pKa is the negative logarithm of the acid dissociation constant (Ka) for a specified proton from a conjugate base.

For example, acidic residues such as aspartic acid and glutamic acid have side chain carboxylic acid groups with pKa values of approximately 4.1 (precise pKa values may depend on temperature, ionic strength and the microenvironment of the ionisable group).

Thus, these side chains exhibit a negative charge at a pH of 7.4 (often referred to as “physiological pH”). At low pH values, these side chains will become protonated and lose their charge.

Conversely, basic residues such as lysine and arginine have nitrogen-containing side chain groups with pKa values of approximately 10-12. These side chains therefore exhibit a positive charge at a pH of 7.4. These side chains will become de-protonated and lose their charge at high pH values.

The overall (net) charge of a protein molecule therefore depends on the number of acidic and basic residues present in the protein (and their degree of surface exposure) and on the surrounding pH. Changing the surrounding pH changes the overall charge on the protein. Accordingly, for every protein there is a given pH at which the number of positive and negative charges is equal and the protein displays no overall net charge. This point is known as the isoelectric point (pl). The isoelectric point is a standard concept in protein biochemistry with which the skilled person would be familiar.

The isoelectric point (pl) is therefore defined as the pH value at which a protein displays a net charge of zero. An increase in pl means that a higher pH value is required for the protein to display a net charge of zero. Thus, an increase in pl represents an increase in the net positive charge of a protein at a given pH. Conversely, a decrease in pl means that a lower pH value is required for the protein to display a net charge of zero. Thus, a decrease in pl represents a decrease in the net positive charge of a protein at a given pH.

Methods of determining the pl of a protein are known in the art and would be familiar to a skilled person. By way of example, the pl of a protein can be calculated from the average pKa values of each amino acid present in the protein (“calculated pl”). Such calculations can be performed using computer programs known in the art, such as the Compute pl/MW Tool from ExPASy (https://web.expasy.org/compute_pi/), which is the preferred method for calculating pl in accordance with the present invention. Comparisons of pl values between different molecules should be made using the same calculation technique/program.

Where appropriate, the calculated pl of a protein can be confirmed experimentally using the technique of isoelectric focusing (“observed pl”). This technique uses electrophoresis to separate proteins according to their pl. Isoelectric focusing is typically performed using a gel that has an immobilised pH gradient. When an electric field is applied, the protein migrates through the pH gradient until it reaches the pH at which it has zero net charge, this point being the pl of the protein. Results provided by isoelectric focusing are typically relatively low-resolution in nature, and thus the present inventors believe that results provided by calculated pl (as described above) are more appropriate to use.

Throughout the present specification, “pl” means “calculated pl” unless otherwise stated.

The pl of a protein may be increased or decreased by altering the number of basic and/or acidic groups displayed on its surface. This can be achieved by modifying one or more amino acids of the protein. For example, an increase in pl may be provided by reducing the number of acidic residues, or by increasing the number of basic residues.

A modified BoNT/A of the invention may have a pl value that is at least 0.2, 0.4, 0.5 or 1 pl units higher than that of an unmodified BoNT/A (e.g. SEQ ID NO: 2). Preferably, a modified BoNT/A may have a pl of at least 6.6, e.g. at least 6.8.

The properties of the 20 standard amino acids are indicated in the table below:

| | | | |
|---|---|---|---|
| Amino Acid | | | Side Chain |
| Aspartic acid | Asp | D | Charged (acidic) |
| Glutamic acid | Glu | E | Charged (acidic) |
| Arginine | Arg | R | Charged (basic) |
| Lysine | Lys | K | Charged (basic) |
| Histidine | His | H | Uncharged (polar) |
| Asparagine | Asn | N | Uncharged (polar) |
| Glutamine | Gln | Q | Uncharged (polar) |
| Serine | Ser | S | Uncharged (polar) |
| Threonine | Thr | T | Uncharged (polar) |
| Tyrosine | Tyr | Y | Uncharged (polar) |
| Methionine | Met | M | Uncharged (polar) |
| Tryptophan | Trp | W | Uncharged (polar) |
| Cysteine | Cys | C | Uncharged (polar) |
| Alanine | Ala | A | Uncharged (hydrophobic) |
| Glycine | Gly | G | Uncharged (hydrophobic) |
| Valine | Val | V | Uncharged (hydrophobic) |
| Leucine | Leu | L | Uncharged (hydrophobic) |
| Isoleucine | Ile | I | Uncharged (hydrophobic) |
| Proline | Pro | P | Uncharged (hydrophobic) |
| Phenylalanine | Phe | F | Uncharged (hydrophobic) |

The following amino acids are considered charged amino acids: aspartic acid (negative), glutamic acid (negative), arginine (positive), and lysine (positive).

At a pH of 7.4, the side chains of aspartic acid (pKa 3.1) and glutamic acid (pKa 4.1) have a negative charge, while the side chains of arginine (pKa 12.5) and lysine (pKa 10.8) have a positive charge. Aspartic acid and glutamic acid are referred to as acidic amino acid residues. Arginine and lysine are referred to as basic amino acid residues.

The following amino acids are considered uncharged, polar (meaning they can participate in hydrogen bonding) amino acids: asparagine, glutamine, histidine, serine, threonine, tyrosine, cysteine, methionine, and tryptophan.

The following amino acids are considered uncharged, hydrophobic amino acids: alanine, valine, leucine, isoleucine, phenylalanine, proline, and glycine.

In an amino acid insertion, an additional amino acid residue (one that is not normally present) is incorporated into the BoNT/A polypeptide sequence, thus increasing the total number of amino acid residues in said sequence. In an amino acid deletion, an amino acid residue is removed from the clostridial toxin amino acid sequence, thus reducing the total number of amino acid residues in said sequence.

Preferably, the modification is a substitution, which advantageously maintains the same number of amino acid residues in the modified BoNT/A. In an amino acid substitution, an amino acid residue that forms part of the BoNT/A polypeptide sequence is replaced with a different amino acid residue. The replacement amino acid residue may be one of the 20 standard amino acids, as described above. Alternatively, the replacement amino acid in an amino acid substitution may be a non-standard amino acid (an amino acid that is not part of the standard set of 20 described above). By way of example, the replacement amino acid may be a basic non-standard amino acid, e.g. L-Ornithine, L-2-amino-3-guanidinopropionic acid, or D-isomers of Lysine, Arginine and Ornithine). Methods for introducing non-standard amino acids into proteins are known in the art and include recombinant protein synthesis using E. coli auxotrophic expression hosts.

In one embodiment, the substitution is selected from: substitution of an acidic amino acid residue with a basic amino acid residue, substitution of an acidic amino acid residue with an uncharged amino acid residue, and substitution of an uncharged amino acid residue with a basic amino acid residue. In one embodiment, wherein the substitution is a substitution of an acidic amino acid residue with an uncharged amino acid residue, the acidic amino acid residue is replaced with its corresponding uncharged amide amino acid residue (i.e. aspartic acid is replaced with asparagine, and glutamic acid is replaced with glutamine).

Preferably, the basic amino acid residue is a lysine residue or an arginine residue. In other words, the substitution is substitution with lysine or arginine. Most preferably, the modification is substitution with lysine.

Following modification in accordance with the invention, the modified BoNT/A is capable of binding to the target cell receptors that unmodified BoNT/A (e.g. SEQ ID NO: 2) binds.

A modified BoNT/A for use in the invention may comprise a BoNT/A light-chain and translocation domain (a BoNT/A LH_{N} domain), and a BoNT/B H_{C} domain. The BoNT/A LH_{N} domain is covalently linked to the BoNT/B H_{C} domain. Said modified BoNT/A is also referred to herein as “BoNT/AB” or a “BoNT/AB chimera”.

The C-terminal amino acid residue of the LH_{N} domain may correspond to the first amino acid residue of the 3₁₀ helix separating the LH_{N} and H_{C} domains of BoNT/A, and the N-terminal amino acid residue of the H_{C} domain may correspond to the second amino acid residue of the 3₁₀ helix separating the LH_{N} and H_{C} domains in BoNT/B.

An example of a BoNT/B polypeptide sequence is provided as SEQ ID NO: 16 (UniProt accession number B1INP5).

Reference herein to the “first amino acid residue of the 3₁₀ helix separating the LH_{N} and H_{C} domains of BoNT/A” means the N-terminal residue of the 3₁₀ helix separating the LH_{N} and H_{C} domains.

Reference herein to the “second amino acid residue of the 3₁₀ helix separating the LH_{N} and H_{C} domains of BoNT/B” means the amino acid residue following the N-terminal residue of the 3₁₀ helix separating the LH_{N} and H_{C} domains.

A “3₁₀ helix” is a type of secondary structure found in proteins and polypeptides, along with α-helices, β-sheets and reverse turns. The amino acids in a 3₁₀ helix are arranged in a right-handed helical structure where each full turn is completed by three residues and ten atoms that separate the intramolecular hydrogen bond between them. Each amino acid corresponds to a 120° turn in the helix (i.e., the helix has three residues per turn), and a translation of 2.0 Å (= 0.2 nm) along the helical axis, and has 10 atoms in the ring formed by making the hydrogen bond. Most importantly, the N-H group of an amino acid forms a hydrogen bond with the C = O group of the amino acid three residues earlier; this repeated i + 3 → i hydrogen bonding defines a 3₁₀ helix. A 3₁₀ helix is a standard concept in structural biology with which the skilled person is familiar.

This 3₁₀ helix corresponds to four residues which form the actual helix and two cap (or transitional) residues, one at each end of these four residues. The term “3₁₀ helix separating the LH_{N} and H_{C} domains” as used herein consists of those 6 residues.

Through carrying out structural analyses and sequence alignments, a 3₁₀ helix separating the LH_{N} and H_{C} domains was identified. This 3₁₀ helix is surrounded by an α-helix at its N-terminus (i.e. at the C-terminal part of the LH_{N} domain) and by a β-strand at its C-terminus (i.e. at the N-terminal part of the H_{C} domain). The first (N-terminal) residue (cap or transitional residue) of the 3₁₀ helix also corresponds to the C-terminal residue of this α-helix.

The 3₁₀ helix separating the LH_{N} and H_{C} domains can be for example determined from publically available crystal structures of botulinum neurotoxins, for example 3BTA (http://www.rcsb.org/pdb/explore/explore.do?structureld=3BTA) and 1EPW (http://www.rcsb.org/pdb/explore/explore.do?structureld=1EPW) for botulinum neurotoxins A1 and B1 respectively.

In silico modelling and alignment tools which are publically available can also be used to determine the location of the 3₁₀ helix separating the LH_{N} and H_{C} domains in other neurotoxins, for example the homology modelling servers LOOPP (Learning, Observing and Outputting Protein Patterns, http://loopp.org), PHYRE (Protein Homology/analogY Recognition Engine, http://www.sbg.bio.ic.ac.uk/phyre2/) and Rosetta (https://www.rosettacommons.org/), the protein superposition server SuperPose (http://wishart.biology.ualberta.ca/superpose/), the alignment program Clustal Omega (http://www.clustal.org/omega/), and a number of other tools/services listed at the Internet Resources for Molecular and Cell Biologists (http://molbiol-tools.ca/). In particular that the region around the “H_{N}/H_{CN}” junction is structurally highly conserved which renders it an ideal region to superimpose different serotypes.

For example, the following methodology was used to determine the sequence of this 3₁₀ helix in other neurotoxins:
1. The structural homology modelling tool LOOP (http://loopp.org) was used to obtain a predicted structure of other BoNT serotypes based on the BoNT/A1 crystal structure (3BTA.pdb);
2. The structural (pdb) files thus obtained were edited to include only the N-terminal end of the H_{CN} domain and about 80 residues before it (which are part of the H_{N} domain), thereby retaining the “H_{N}/H_{CN}” region which is structurally highly conserved;
3. The protein superposition server SuperPose (http://wishart.biology.ualberta.ca/superpose/) was used to superpose each serotype onto the 3BTA.pdb structure;
4. The superposed pdb files were inspected to locate the 3₁₀ helix at the start of the H_{C} domain of BoNT/A1, and corresponding residues in the other serotype were then identified.
5. The other BoNT serotype sequences were aligned with Clustal Omega in order to check that corresponding residues were correct.

Examples of LH_{N}, H_{C} and 3₁₀ helix domains determined by this method are presented below:

| Neurotoxin | Accession Number (Plus Sequence Version after Decimal) | LH_{N} | H_{C} | 3₁₀ helix |
|---|---|---|---|---|
| BoNT/A1 (SEQ ID NO: 2) | A5HZZ9.1 | 1-872 | 873-1296 | ⁸⁷²NIINTS⁸⁷⁷ |
| BoNT/A2 | X73423.3 | 1-872 | 873-1296 | ⁸⁷²NIVNTS⁸⁷⁷ |
| BoNT/A3 | DQ185900.1 (aka Q3LRX9.1) | 1-872 | 873-1292 | ⁸⁷²NIVNTS⁸⁷⁷ |
| BoNT/A4 | EU341307.1 (aka Q3LRX8.1) | 1-872 | 873-1296 | ⁸⁷²NITNAS⁸⁷⁷ |
| BoNT/A5 | EU679004.1 (aka C1IPK2.1) | 1-872 | 873-1296 | ⁸⁷²NIINTS⁸⁷⁷ |
| BoNT/A6 | FJ981696.1 | 1-872 | 873-1296 | ⁸⁷²NIINTS⁸⁷⁷ |
| BoNT/A7 | JQ954969.1 (aka K4LN57.1) | 1-872 | 873-1296 | ⁸⁷²NIINTS⁸⁷⁷ |
| BoNT/A8 | KM233166.1 | 1-872 | 873-1297 | ⁸⁷²NITNTS⁸⁷⁷ |
| BoNT/B1 (SEQ ID NO: 16) | B1lNP5.1 | 1-859 | 860-1291 | ⁸⁵⁹EILNNI⁸⁶⁴ |
| BoNT/B2 | AB084152.1 (aka Q8GR96.1) | 1-859 | 860-1291 | ⁸⁵⁹EILNNI⁸⁶⁴ |
| BoNT/B3 | EF028400.1 (aka A2I2S2.1 ) | 1-859 | 860-1291 | ⁸⁵⁹EILNNI⁸⁶⁴ |
| BoNT/B4 | EF051570.1 (aka A2I2W0.1) | 1-859 | 860-1291 | ⁸⁵⁹EILNNI⁸⁶⁴ |
| BoNT/B5 | EF033130.1 (aka A2I2U6.1) | 1-859 | 860-1291 | ⁸⁵⁹DILNNI⁸⁶⁴ |
| BoNT/B6 | AB302852.1 (aka A8R089.1) | 1-859 | 860-1291 | ⁸⁵⁹EILNNI⁸⁶⁴ |
| BoNT/B7 | JQ354985.1 (aka H9CNK9.1) | 1-859 | 860-1291 | ⁸⁵⁹EILNNI⁸⁶⁴ |
| BoNT/B8 | JQ964806.1 (aka I6Z8G9.1) | 1-859 | 860-1292 | ⁸⁵⁹EILNNI⁸⁶⁴ |

Using structural analysis and sequence alignments, it was found that the β-strand following the 3₁₀ helix separating the LH_{N} and H_{C} domains is a conserved structure in all botulinum and tetanus neurotoxins and starts at the 8^{th} residue when starting from the first residue of the 3₁₀ helix separating the LH_{N} and H_{C} domains (e.g., at residue 879 for BoNT/A1).

A BoNT/AB chimera may comprise an LH_{N} domain from BoNT/A covalently linked to a H_{C} domain from BoNT/B,
wherein the C-terminal amino acid residue of the LH_{N} domain corresponds to the eighth amino acid residue N-terminally to the β-strand located at the beginning (N-term) of the H_{C} domain of BoNT/A, and
wherein the N-terminal amino acid residue of the H_{C} domain corresponds to the seventh amino acid residue N-terminally to the β-strand located at the beginning (N-term) of the H_{C} domain of BoNT/B.

A BoNT/AB chimera may comprise an LH_{N} domain from BoNT/A covalently linked to a H_{C} domain from BoNT/B,
wherein the C-terminal amino acid residue of the LH_{N} domain corresponds to the C-terminal amino acid residue of the α-helix located at the end (C-term) of LH_{N} domain of BoNT/A, and
wherein the N-terminal amino acid residue of the H_{C} domain corresponds to the amino acid residue immediately C-terminal to the C-terminal amino acid residue of the α-helix located at the end (C-term) of LH_{N} domain of BoNT/B.

The rationale of the design process of the BoNT/AB chimera is to try to ensure that the secondary structure was not compromised and thereby minimise any changes to the tertiary structure and to the function of each domain. Without wishing to be bound by theory, it is hypothesized that by not disrupting the four central amino acid residues of the 3₁₀ helix in the BoNT/AB chimera ensures an optimal conformation for the chimeric neurotoxin, thereby allowing for the chimeric neurotoxin to exert its functions to their full capacity.

In fact, surprisingly, retaining solely the first amino acid residue of the 3₁₀ helix of the BoNT/A and the second amino acid residue of the 3₁₀ helix onwards of BoNT/B not only allows the production of soluble and functional BoNT/AB chimera, but further leads to improved properties over other BoNT/AB chimeras, in particular an increased potency, an increased Safety Ratio and/or a longer duration of action (as well as increased Safety Ratio and/or duration of action when compared to unmodified BoNT/A).

The LH_{N} domain from BoNT/A may correspond to amino acid residues 1 to 872 of SEQ ID NO: 2, or a polypeptide sequence having at least 70% sequence identity thereto. The LH_{N} domain from BoNT/A may correspond to amino acid residues 1 to 872 of SEQ ID NO: 2, or a polypeptide sequence having at least 80%, 90% or 95% sequence identity thereto. Preferably, the LH_{N} domain from BoNT/A corresponds to amino acid residues 1 to 872 of SEQ ID NO: 2.

The H_{C} domain from BoNT/B may correspond to amino acid residues 860 to 1291 of SEQ ID NO: 16, or a polypeptide sequence having at least 70% sequence identity thereto. The H_{C} domain from BoNT/B may correspond to amino acid residues 860 to 1291 of SEQ ID NO: 16, or a polypeptide sequence having at least 80%, 90% or 95% sequence identity thereto. Preferably, the H_{C} domain from BoNT/B corresponds to amino acid residues 860 to 1291 of SEQ ID NO: 16.

Preferably, the BoNT/AB chimera comprises a BoNT/A1 LH_{N} domain and a BoNT/B1 H_{C} domain. More preferably, the LH_{N} domain corresponds to amino acid residues 1 to 872 of BoNT/A1 (SEQ ID NO: 2) and the H_{C} domain corresponds to amino acid residues 860 to 1291 of BoNT/B1 (SEQ ID NO: 16).

Preferably, a BoNT/B H_{C} domain further comprises at least one amino acid residue substitution, addition or deletion in the H_{CC} subdomain which has the effect of increasing the binding affinity of BoNT/B neurotoxin for human Syt II as compared to the natural BoNT/B sequence. Suitable amino acid residue substitution, addition or deletion in the BoNT/B H_{CC} subdomain have been disclosed in WO 2013/180799 and in WO 2016/154534 (both herein incorporated by reference).

Suitable amino acid residue substitution, addition or deletion in the BoNT/B H_{CC} subdomain include substitution mutations selected from the group consisting of: V1118M; Y1183M; E1191M; E1191I; E1191Q; E1191T; S1199Y; S1199F; S1199L; S1201V; E1191C, E1191V, E1191L, E1191Y, S1199W, S1199E, S1199H, W1178Y, W1178Q, W1178A, W1178S, Y1183C, Y1183P and combinations thereof.

Suitable amino acid residue substitution, addition or deletion in the BoNT/B H_{CC} subdomain further include combinations of two substitution mutations selected from the group consisting of: E1191M and S1199L, E1191M and S1199Y, E1191M and S1199F, E1191Q and S1199L, E1191Q and S1199Y, E1191Q and S1199F, E1191M and S1199W, E1191M and W1178Q, E1191C and S1199W, E1191C and S1199Y, E1191C and W1178Q, E1191Q and S1199W, E1191V and S1199W, E1191V and S1199Y, or E1191V and W1178Q.

Suitable amino acid residue substitution, addition or deletion in the BoNT/B H_{CC} subdomain also include a combination of three substitution mutations which are E1191M, S1199W and W1178Q.

Preferably, the suitable amino acid residue substitution, addition or deletion in the BoNT/B H_{CC} subdomain includes a combination of two substitution mutations which are E1191M and S1199Y. Such modifications are present in BoNT/AB chimeras SEQ ID NO: 13 and SEQ ID NO: 14.

The modification may be a modification when compared to unmodified BoNT/B shown as SEQ ID NO: 16, wherein the amino acid residue numbering is determined by alignment with SEQ ID NO: 16. As the presence of a methionine residue at position 1 of SEQ ID NO: 16 (as well as the SEQ ID NOs corresponding to modified BoNT/A polypeptides described herein) is optional, the skilled person will take the presence/absence of the methionine residue into account when determining amino acid residue numbering. For example, where SEQ ID NO: 16 includes a methionine, the position numbering will be as defined above (e.g. E1191 will be E1191 of SEQ ID NO: 16). Alternatively, where the methionine is absent from SEQ ID NO: 16 the amino acid residue numbering should be modified by -1 (e.g. E1191 will be E1190 of SEQ ID NO: 16). Similar considerations apply when the methionine at position 1 of the other polypeptide sequences described herein is present/absent, and the skilled person will readily determine the correct amino acid residue numbering using techniques routine in the art.

A modified BoNT/A for use in the invention may comprise a polypeptide sequence having at least 70% sequence identity to a polypeptide sequence selected from SEQ ID NOs: 11-15. For example, a polypeptide sequence having at least 80%, 90%, 95% or 99.9% sequence identity to a polypeptide sequence selected from SEQ ID NOs: 11-15. Preferably, a modified BoNT/A for use in the invention may comprise (more preferably consist of) a polypeptide sequence selected from SEQ ID NOs: 11-15.

When a modified BoNT/A is a BoNT/AB chimera, it is preferred that the modified BoNT/A comprises a polypeptide sequence having at least 70% sequence identity to SEQ ID NO: 14. For example, a polypeptide sequence having at least 80%, 90%, 95% or 99.9% sequence identity to SEQ ID NO: 14. Preferably, a modified BoNT/A for use in the invention may comprise (more preferably consist of) SEQ ID NO: 14.

Methods for modifying proteins by substitution, insertion or deletion of amino acid residues are known in the art. By way of example, amino acid modifications may be introduced by modification of a DNA sequence encoding a BoNT/A (e.g. encoding unmodified BoNT/A). This can be achieved using standard molecular cloning techniques, for example by site-directed mutagenesis where short strands of DNA (oligonucleotides) coding for the desired amino acid(s) are used to replace the original coding sequence using a polymerase enzyme, or by inserting/deleting parts of the gene with various enzymes (e.g., ligases and restriction endonucleases). Alternatively, a modified gene sequence can be chemically synthesised.

As discussed above, a modified BoNT/A described herein has increased tissue retention properties that also provide increased potency and/or duration of action and can allow for increased dosages without any additional negative effects. One way in which these advantageous properties (which represent an increase in the therapeutic index) may be defined is in terms of the Safety Ratio of the modified BoNT/A. In this regard, undesired effects of a clostridial toxin (caused by diffusion of the toxin away from the site of administration) can be assessed experimentally by measuring percentage bodyweight loss in a relevant animal model (e.g. a mouse, where loss of bodyweight is detected within seven days of administration). Conversely, desired on-target effects of a clostridial toxin can be assessed experimentally by Digital Abduction Score (DAS) assay, a measurement of muscle paralysis. The DAS assay may be performed by injection of 20 µl of clostridial toxin, formulated in Gelatin Phosphate Buffer, into the mouse gastrocnemius/soleus complex, followed by assessment of Digital Abduction Score using the method of Aoki (Aoki KR, Toxicon 39: 1815-1820; 2001). In the DAS assay, mice are suspended briefly by the tail in order to elicit a characteristic startle response in which the mouse extends its hind limbs and abducts its hind digits. Following clostridial toxin injection, the varying degrees of digit abduction are scored on a five-point scale (0=normal to 4=maximal reduction in digit abduction and leg extension).

The Safety Ratio of a modified BoNT/A of the invention (or unmodified BoNT/A for comparison) may then be expressed as the ratio between the amount of toxin required for a 10% drop in a bodyweight (measured at peak effect within the first seven days after dosing in a mouse) and the amount of toxin required for a DAS score of 2. High Safety Ratio scores are therefore desired and indicate a toxin that is able to effectively paralyse a target muscle with little undesired off-target effects. A modified BoNT/A of the present invention has a Safety Ratio that is higher than the Safety Ratio of an equivalent unmodified (native) BoNT/A.

Thus, in one embodiment, a modified BoNT/A of the present invention has a Safety Ratio that is greater than 7 (for example, at least 8, 9, 10, 15, 20, 25, 30, 35, 40, 45 or 50), wherein Safety Ratio is calculated as: dose of toxin required for -10% bodyweight change (pg/mouse) divided by DAS ED₅₀ (pg/mouse) [ED₅₀ = dose required to produce a DAS score of 2].

In one embodiment, a modified BoNT/A of the present invention has a Safety Ratio of at least 10. In one embodiment, a modified BoNT/A of the present invention has a Safety Ratio of at least 15.

Preferably, where a modified BoNT/A is one comprising one or more amino acid residue(s) selected from: ASN 886, ASN 930, ASN 954, SER 955, GLN 991, ASN 1025, ASN 1026, ASN 1052, ASN 1188, ASP 1213, GLY 1215, ASN 1216, GLN 1229, ASN 1242, ASN 1243, SER 1274 and THR 1277 as described herein, said modified BoNT/A has a Safety Ratio of at least 20, more preferably at least 22 (e.g. 23-25).

Preferably, where a modified BoNT/A is one that comprises a BoNT/A light-chain and translocation domain, and a BoNT/B H_{C} domain, the modified BoNT/A has a Safety Ratio of at least 10, more preferably at least 12 (e.g. 14-15).

In use, the modified BoNT/A of the invention is in a di-chain form.

The modified BoNT/A is preferably in a non-complexed form (i.e. free from complexing proteins that are present in naturally occurring BoNT/A). Examples of such complexing proteins include a neurotoxin-associated proteins (NAP) and a nontoxic-nonhemagglutinin component (NTNH). It is preferred that the modified BoNT/A is a recombinant modified BoNT/A.

The modified BoNT/A may be produced by a method of producing a single-chain modified BoNT/A having a light chain and a heavy chain, the method comprising expressing a nucleic acid (said nucleic acid being as described above) in a suitable host cell, lysing the host cell to provide a host cell homogenate containing the single-chain modified BoNT/A, and isolating the single-chain modified BoNT/A. The modified BoNT/A may then be activated by a method comprising providing a single-chain modified BoNT/A protein obtainable by the method of producing a single-chain modified BoNT/A as described above, contacting the modified BoNT/A with a protease that cleaves the polypeptide at a recognition site (cleavage site) located between the light chain and heavy chain, thereby converting the polypeptide into a di-chain modified BoNT/A wherein the light chain and heavy chain are joined together by a disulphide bond.

The modified BoNT/A of the invention may be formulated in any suitable manner for administration to a subject, for example as part of a pharmaceutical composition. Thus, in one aspect, the invention provides a pharmaceutical composition comprising a modified BoNT/A of the invention and a pharmaceutically acceptable carrier, excipient, adjuvant, and/or salt.

In one aspect, the invention provides a unit dosage form of modified botulinum neurotoxin A (BoNT/A), the unit dosage form comprising:
a. 1 Units to 41 Units (2 Units to 41 Units) of modified BoNT/A, wherein 1 Unit is an amount of the modified BoNT/A that corresponds to the calculated median lethal dose (LD₅₀) in mice; or
b. 8.4 pg to 350 pg (18 pg to 350 pg) of modified BoNT/A; and
c. optionally a pharmaceutically acceptable carrier, excipient, adjuvant, and/or salt,
   wherein the modified BoNT/A comprises a modification at one or more amino acid residue(s) selected from: ASN 886, ASN 905, GLN 915, ASN 918, GLU 920, ASN 930, ASN 954, SER 955, GLN 991, GLU 992, GLN 995, ASN 1006, ASN 1025, ASN 1026, ASN 1032, ASN 1043, ASN 1046, ASN 1052, ASP 1058, HIS 1064, ASN 1080, GLU 1081, GLU 1083, ASP 1086, ASN 1188, ASP 1213, GLY 1215, ASN 1216, GLN 1229, ASN 1242, ASN 1243, SER 1274, and THR 1277, wherein the modification is selected from:
      i. substitution of an acidic surface exposed amino acid residue with a basic amino acid residue;
      ii. substitution of an acidic surface exposed amino acid residue with an uncharged amino acid residue;
      iii. substitution of an uncharged surface exposed amino acid residue with a basic amino acid residue;
      iv. insertion of a basic amino acid residue; and
      v. deletion of an acidic surface exposed amino acid residue.

In one embodiment, the invention provides a unit dosage form comprising:
a. 1 Unit to 35 Units of modified BoNT/A; or
b. 10 pg to 300 pg of modified BoNT/A.

In one aspect, the invention provides a unit dosage form of modified botulinum neurotoxin A (BoNT/A), the unit dosage form comprising:
a. 2 Units to 41 Units of modified BoNT/A, wherein 1 Unit is an amount of the modified BoNT/A that corresponds to the calculated median lethal dose (LD₅₀) in mice; or
b. 18 pg to 350 pg of modified BoNT/A; and
c. optionally a pharmaceutically acceptable carrier, excipient, adjuvant, and/or salt,
wherein the modified BoNT/A comprises a BoNT/A light-chain and translocation domain, and a BoNT/B receptor binding domain (H_{C} domain).

In one aspect, the invention provides a unit dosage form of modified botulinum neurotoxin A (BoNT/A), the unit dosage form comprising:
a. 0.5 Units to 73 Units of modified BoNT/A, wherein 1 Unit is an amount of the modified BoNT/A that corresponds to the calculated median lethal dose (LD₅₀) in mice; or
b. 12 pg to 1754 pg of modified BoNT/A; and
c. optionally a pharmaceutically acceptable carrier, excipient, adjuvant, and/or salt,
wherein the modified BoNT/A comprises a BoNT/A light-chain and translocation domain, and a BoNT/B receptor binding domain (H_{C} domain).

The invention provides a unit dosage form comprising:
a. 0.8 Units to 62 Units of modified BoNT/A; or
b. 20 pg to 1500 pg of modified BoNT/A.

A unit dosage form may comprise 6 Units to 35 Units, 12 Units to 35 Units, 12 Units to 24 Units, preferably 12 Units to 18 Units of modified BoNT/A.

A unit dosage form may comprise 50 pg to 300 pg, 100 to 300 pg, 100 to 200 pg, preferably 100 to 150 pg of modified BoNT/A.

A kit comprising:
a. the unit dosage form as hereinbefore defined; and
b. instructions for use of the same in treating facial lines; and
c. optionally a diluent.

Embodiments related to the various therapeutic uses of the invention can be applied to the methods, compositions (e.g. unit dosage forms), and kits of the invention and vice versa.

### Sequence Homology

Any of a variety of sequence alignment methods can be used to determine percent identity, including, without limitation, global methods, local methods and hybrid methods, such as, e.g., segment approach methods. Protocols to determine percent identity are routine procedures within the scope of one skilled in the art. Global methods align sequences from the beginning to the end of the molecule and determine the best alignment by adding up scores of individual residue pairs and by imposing gap penalties. Non-limiting methods include, e.g., CLUSTAL W, see, e.g., Julie D. Thompson et al., CLUSTAL W: Improving the Sensitivity of Progressive Multiple Sequence Alignment Through Sequence Weighting, Position- Specific Gap Penalties and Weight Matrix Choice, 22(22) Nucleic Acids Research 4673-4680 (1994); and iterative refinement, see, e.g., Osamu Gotoh, Significant Improvement in Accuracy of Multiple Protein. Sequence Alignments by Iterative Refinement as Assessed by Reference to Structural Alignments, 264(4) J. Mol. Biol. 823-838 (1996). Local methods align sequences by identifying one or more conserved motifs shared by all of the input sequences. Non-limiting methods include, e.g., Match-box, see, e.g., Eric Depiereux and Ernest Feytmans, Match-Box: A Fundamentally New Algorithm for the Simultaneous Alignment of Several Protein Sequences, 8(5) CABIOS 501 -509 (1992); Gibbs sampling, see, e.g., C. E. Lawrence et al., Detecting Subtle Sequence Signals: A Gibbs Sampling Strategy for Multiple Alignment, 262(5131 ) Science 208-214 (1993); Align-M, see, e.g., Ivo Van Walle et al., Align-M - A New Algorithm for Multiple Alignment of Highly Divergent Sequences, 20(9) Bioinformatics:1428-1435 (2004).

Thus, percent sequence identity is determined by conventional methods. See, for example, Altschul et al., Bull. Math. Bio. 48: 603-16, 1986 and Henikoff and Henikoff, Proc. Natl. Acad. Sci. USA 89:10915-19, 1992. Briefly, two amino acid sequences are aligned to optimize the alignment scores using a gap opening penalty of 10, a gap extension penalty of 1, and the “blosum 62” scoring matrix of Henikoff and Henikoff (ibid.) as shown below (amino acids are indicated by the standard one-letter codes).

The “percent sequence identity” between two or more nucleic acid or amino acid sequences is a function of the number of identical positions shared by the sequences. Thus, % identity may be calculated as the number of identical nucleotides / amino acids divided by the total number of nucleotides / amino acids, multiplied by 100. Calculations of % sequence identity may also take into account the number of gaps, and the length of each gap that needs to be introduced to optimize alignment of two or more sequences. Sequence comparisons and the determination of percent identity between two or more sequences can be carried out using specific mathematical algorithms, such as BLAST, which will be familiar to a skilled person.

### Alignment Scores for Determining Sequence Identity

A R N D C Q E G H I L K M F P S T W Y V

$$\begin{matrix}\text{A 4} \\ \text{R} \text{-1} \text{5} \\ \text{N} \text{-2} \text{0} \text{6} \\ \text{D} \text{-2 -2 1 6} \\ \text{C 0 -3 -3 -3 9} \\ \text{Q -1 1 0 0 -3 5} \\ \text{E -1 0 0 2 -4 2 5} \\ \text{G 0 -2 0 -1 -3 -2 -2 6} \\ \text{H -2 0 1 -1 -3 0 0 -2 8} \\ \text{I -1 -3 -3 -3 -1 -3 -3 -4 -3 4} \\ \text{L -1 -2 -3 -4 -1 -2 -3 -4 -3 2 4} \\ \text{K -1 2 0 -1 -3 1 1 -2 -1 -3 -2 5} \\ \text{M -1 -1 -2 -3 -1 0 -2 -3 -2 1 2 -1 5} \\ \text{F -2 -3 -3 -3 -2 -3 -3 -3 -1 0 0 -3 0 6} \\ \text{P -1 -2 -2 -1 -3 -1 -1 -2 -2 -3 -3 -1 -2 -4 7} \\ \text{S 1 -1 1 0 -1 0 0 0 -1 -2 -2 0 -1 -2 -1 4} \\ \text{T 0 -1 0 -1 -1 -1 -1 -2 -2 -1 -1 -1 -1 -2 -1 1 5} \\ \text{W -3 -3 -4 -4 -2 -2 -3 -2 -2 -3 -2 -3 -1 1 -4 -3 -2 11} \\ \text{Y -2 -2 -2 -3 -2 -1 -2 -3 2 -1 -1 -2 -1 3 -3 -2 -2 2 7} \\ \text{V 0 -3 -3 -3 -1 -2 -2 -3 -3 3 1 -2 1 -1 -2 -2 0 -3 -1 4}\end{matrix}$$

The percent identity is then calculated as:

$$\frac{\text{Total number of identical matches}}{\begin{matrix}\left[\text{length of the longer sequence plus the}\right) \\ \text{number of gaps introduced into the longer} \\ \left(\text{sequence in order to align the two sequences}\right]\end{matrix}} \times 100$$

Substantially homologous polypeptides are characterized as having one or more amino acid substitutions, deletions or additions. These changes are preferably of a minor nature, that is conservative amino acid substitutions (see below) and other substitutions that do not significantly affect the folding or activity of the polypeptide; small deletions, typically of one to about 30 amino acids; and small amino- or carboxyl-terminal extensions, such as an amino-terminal methionine residue, a small linker peptide of up to about 20-25 residues, or an affinity tag.

CONSERVATIVE AMINO ACID SUBSTITUTIONS

| Basic: | arginine |
|---|---|
| | lysine |
| | histidine |
| Acidic: | glutamic acid |
| | aspartic acid |
| Polar: | glutamine |
| | asparagine |
| Hydrophobic: | leucine |
| | isoleucine |
| | valine |
| Aromatic: | phenylalanine |
| | tryptophan |
| | tyrosine |
| Small: | glycine |
| | alanine |
| | serine |
| | threonine |
| | methionine |

In addition to the 20 standard amino acids, non-standard amino acids (such as 4-hydroxyproline, 6-N-methyl lysine, 2-aminoisobutyric acid, isovaline and α -methyl serine) may be substituted for amino acid residues of the polypeptides of the present invention. A limited number of non-conservative amino acids, amino acids that are not encoded by the genetic code, and unnatural amino acids may be substituted for polypeptide amino acid residues. The polypeptides of the present invention can also comprise non-naturally occurring amino acid residues.

Non-naturally occurring amino acids include, without limitation, trans-3-methylproline, 2,4-methano-proline, cis-4-hydroxyproline, trans-4-hydroxy-proline, N-methylglycine, allo-threonine, methyl-threonine, hydroxy-ethylcysteine, hydroxyethylhomo-cysteine, nitro-glutamine, homoglutamine, pipecolic acid, tert-leucine, norvaline, 2-azaphenylalanine, 3-azaphenyl-alanine, 4-azaphenyl-alanine, and 4-fluorophenylalanine. Several methods are known in the art for incorporating non-naturally occurring amino acid residues into proteins. For example, an in vitro system can be employed wherein nonsense mutations are suppressed using chemically aminoacylated suppressor tRNAs. Methods for synthesizing amino acids and aminoacylating tRNA are known in the art. Transcription and translation of plasmids containing nonsense mutations is carried out in a cell free system comprising an E. coli S30 extract and commercially available enzymes and other reagents. Proteins are purified by chromatography. See, for example, Robertson et al., J. Am. Chem. Soc. 113:2722, 1991; Ellman et al., Methods Enzymol. 202:301, 1991; Chung et al., Science 259:806-9, 1993; and Chung et al., Proc. Natl. Acad. Sci. USA 90:10145-9, 1993). In a second method, translation is carried out in Xenopus oocytes by microinjection of mutated mRNA and chemically aminoacylated suppressor tRNAs (Turcatti et al., J. Biol. Chem. 271:19991-8, 1996). Within a third method, E. coli cells are cultured in the absence of a natural amino acid that is to be replaced (e.g., phenylalanine) and in the presence of the desired non-naturally occurring amino acid(s) (e.g., 2-azaphenylalanine, 3-azaphenylalanine, 4-azaphenylalanine, or 4-fluorophenylalanine). The non-naturally occurring amino acid is incorporated into the polypeptide in place of its natural counterpart. See, Koide et al., Biochem. 33:7470-6, 1994. Naturally occurring amino acid residues can be converted to non-naturally occurring species by in vitro chemical modification. Chemical modification can be combined with site-directed mutagenesis to further expand the range of substitutions (Wynn and Richards, Protein Sci. 2:395-403, 1993).

A limited number of non-conservative amino acids, amino acids that are not encoded by the genetic code, non-naturally occurring amino acids, and unnatural amino acids may be substituted for amino acid residues of polypeptides of the present invention.

Essential amino acids in the polypeptides of the present invention can be identified according to procedures known in the art, such as site-directed mutagenesis or alanine-scanning mutagenesis (Cunningham and Wells, Science 244: 1081-5, 1989). Sites of biological interaction can also be determined by physical analysis of structure, as determined by such techniques as nuclear magnetic resonance, crystallography, electron diffraction or photoaffinity labeling, in conjunction with mutation of putative contact site amino acids. See, for example, de Vos et al., Science 255:306-12, 1992; Smith et al., J. Mol. Biol. 224:899-904, 1992; Wlodaver et al., FEBS Lett. 309:59-64, 1992. The identities of essential amino acids can also be inferred from analysis of homologies with related components (e.g. the translocation or protease components) of the polypeptides of the present invention.

Multiple amino acid substitutions can be made and tested using known methods of mutagenesis and screening, such as those disclosed by Reidhaar-Olson and Sauer (Science 241:53-7, 1988) or Bowie and Sauer (Proc. Natl. Acad. Sci. USA 86:2152-6, 1989). Briefly, these authors disclose methods for simultaneously randomizing two or more positions in a polypeptide, selecting for functional polypeptide, and then sequencing the mutagenized polypeptides to determine the spectrum of allowable substitutions at each position. Other methods that can be used include phage display (e.g., Lowman et al., Biochem. 30:10832-7, 1991; Ladner et al., U.S. Pat. No. 5,223,409; Huse, WIPO Publication WO 92/06204) and region-directed mutagenesis (Derbyshire et al., Gene 46:145, 1986; Ner et al., DNA 7:127, 1988).

Unless defined otherwise, all technical and scientific terms used herein have the same meaning as commonly understood by one of ordinary skill in the art to which this disclosure belongs. Singleton, et al., DICTIONARY OF MICROBIOLOGY AND MOLECULAR BIOLOGY, 20 ED., John Wiley and Sons, New York (1994), and Hale & Marham, THE HARPER COLLINS DICTIONARY OF BIOLOGY, Harper Perennial, NY (1991) provide the skilled person with a general dictionary of many of the terms used in this disclosure.

This disclosure is not limited by the exemplary methods and materials disclosed herein, and any methods and materials similar or equivalent to those described herein can be used in the practice or testing of embodiments of this disclosure. Numeric ranges are inclusive of the numbers defining the range. Unless otherwise indicated, any nucleic acid sequences are written left to right in 5′ to 3′ orientation; amino acid sequences are written left to right in amino to carboxy orientation, respectively.

The headings provided herein are not limitations of the various aspects or embodiments of this disclosure.

Amino acids are referred to herein using the name of the amino acid, the three letter abbreviation or the single letter abbreviation. The term “protein”, as used herein, includes proteins, polypeptides, and peptides. As used herein, the term “amino acid sequence” is synonymous with the term “polypeptide” and/or the term “protein”. In some instances, the term “amino acid sequence” is synonymous with the term “peptide”. In some instances, the term “amino acid sequence” is synonymous with the term “enzyme”. The terms “protein” and “polypeptide” are used interchangeably herein. In the present disclosure and claims, the conventional one-letter and three-letter codes for amino acid residues may be used. The 3-letter code for amino acids as defined in conformity with the IUPACIUB Joint Commission on Biochemical Nomenclature (JCBN). It is also understood that a polypeptide may be coded for by more than one nucleotide sequence due to the degeneracy of the genetic code.

Other definitions of terms may appear throughout the specification. Before the exemplary embodiments are described in more detail, it is to be understood that this disclosure is not limited to particular embodiments described, and as such may vary. It is also to be understood that the terminology used herein is for the purpose of describing particular embodiments only, and is not intended to be limiting, since the scope of the present disclosure will be defined only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

It must be noted that as used herein and in the appended claims, the singular forms “a”, “an”, and “the” include plural referents unless the context clearly dictates otherwise. Thus, for example, reference to “a botulinum neurotoxin A” includes a plurality of such candidate agents and reference to “the botulinum neurotoxin A” includes reference to one or more clostridial neurotoxins and equivalents thereof known to those skilled in the art, and so forth.

The publications discussed herein are provided solely for their disclosure prior to the filing date of the present application. Nothing herein is to be construed as an admission that such publications constitute prior art to the claims appended hereto.

### BRIEF DESCRIPTION OF THE DRAWINGS

Embodiments of the invention will now be described, by way of example only, with reference to the following Figures and Examples.

FIG. 1 shows the FDA approved dosages of Dysport® for treating facial lines in adults.

FIG. 2 shows the isoelectric focusing (IEF) gel of cationic constructs.

FIG. 3 shows the percentage SNAP-25 cleavage in rat embryonic spinal cord neurons (eSCN) for Cat5v2(K1064H/N954K) (A), Cat5v2(K1064H/N886K) (B) and Cat5v2(K1064H/ N1025K) (C), and summary of pEC50 relative to nBoNT/A1. (A, B, C) Rat embryonic spinal cord neurons were cultured for three weeks and treated with Cat5v4 for 24 h, before Western blotting with SNAP-25 specific antibody. Data is mean ±SEM from three independent experiments in triplicate. (D) Relative potency of Cat5v2(K1064H/N886K), Cat5v2(K1064H/N954K) and Cat5v2(K1064H/ N1025K) to nBoNT/A1 (List Biological Laboratories) in the rat eSCN SNAP-25 cleavage potency assay. Each point corresponds to an individual batch and is a mean of 3 independent pEC50 determinations based on an 8-point concentration response curve (CRC). Each concentration in the CRC was assessed in triplicate. Potency comparisons are made to a mean of List batches, pooled data n=24. Data are mean ±SEM of n=3 batches per Cat5v4.

FIG. 4 shows the potency (t₅₀) of nBoNT/A1 and Cat5v4 in the mouse phrenic nerve hemi-diaphragm assay (mPNHD). Mouse phrenic nerve hemi-diaphragm tissue was incubated with Cat5v4 or native BoNT/A1 as indicated. Diaphragm contractile force was recorded until the contraction was no longer detectable or after 140 minutes. Each point corresponds to independent determinations. The t₅₀ value is the time required to inhibit the contractile force of the mouse hemi-diaphragm by 50%.

FIG. 5 shows SDS-PAGE of purified recombinant BoNT/AB chimera 1, 2 and 3A (SEQ ID NO: 11, 12 and 13 respectively). Lanes are labelled “Marker” (molecular weight marker), “-DTT” (oxidised BoNT/AB chimera sample), and “+DTT” (reduced BoNT/AB chimera sample).

FIG. 6 shows cleavage of SNAP-25 in rat spinal cord neurones by recombinant BoNT/AB chimera 1, 2 and 3A (SEQ ID NO: 11, 12 and 13 respectively). Cultured rat primary spinal cord neurons (SCN) were exposed to various concentrations of recombinant BoNT/AB chimera 1, 2 or 3A for 24 hours, at 37° C. in a humidified atmosphere with 10% CO₂. Cells were then lysed with 1x NuPAGE buffer supplemented with DTT and Benzonase. The samples were transferred to microcentrifuge tubes, heated for 5 min at 90° C. on heat block and stored at -20° C., before analysis of SNAP-25 cleavage by Western blot. SNAP-25 was detected using a polyclonal antibody, that detects both the full length and cleaved forms of SNAP-25 (Sigma #S9684). Anti-rabbit HRP (Sigma #A6154) was used as the secondary antibody.

FIG. 7 shows mouse digit abduction scoring assay. Mice were injected into the gastrocnemius-soleus complex muscles of one hind limb, under short general anaesthesia; muscle weakening was measured on a 0-4 scale using the digit abduction score (DAS). DAS max values were determined for each dose and plotted against dose and the data were fitted to a 4-parameter logistic equation, ED50 and dose leading to DAS 4 (DAS 4 dose) values were determined.

FIG. 8 shows SDS-PAGE of purified recombinant BoNT/AB chimera 3B and 3C (SEQ ID NO: 14 and 15 respectively). Lanes are labelled “Marker” (molecular weight marker), “-DTT” (oxidised BoNT/AB chimera sample), and “+DTT” (reduced BoNT/AB chimera sample).

FIG. 9 shows cleavage of SNAP-25 by unmodified BoNT/A and BoNT/AB chimera 3B and 3C (SEQ ID NO: 2, 14 and 15 respectively) in human induced pluripotent stem cell derived peripheral neurons (PERI.4U - Axiogenesis, Germany). PERI.4U cells were exposed to various concentrations of recombinant BoNT/A, or BoNT/AB chimera 3B or 3C for 24 hours, at 37° C. in a humidified CO₂ atmosphere containing 5% CO₂. Cells were then lysed with 1x NuPAGE buffer supplemented with DTT and Benzonase. The samples were transferred to microcentrifuge tubes, heated for 5 min at 90° C. on heat block and stored at -20° C., before analysis of SNAP-25 cleavage by Western blot. SNAP-25 was detected using a polyclonal antibody, that detects both the full length and cleaved forms of SNAP-25 (Sigma #S9684). Anti-rabbit HRP (Sigma #A6154) was used as the secondary antibody.

FIG. 10 shows duration of muscle weakening over time in the mouse digit abduction scoring assay. Mice were injected into the gastrocnemius-soleus complex muscles of one hind limb, under short general anaesthesia; muscle weakening was measured on a 0-4 scale using the digit abduction score (DAS). Animals of the group injected with the lowest dose that induced during the first four days of injection a DAS of 4 were monitored until complete recovery of the muscle weakness to a DAS of 0 (no observed muscle weakness).

FIG. 11 shows the injection sites for the intramuscular administration of modified BoNT/A when treating glabellar lines.

FIG. 12 shows the injection sites for the intramuscular administration of modified BoNT/A when treating glabellar lines and forehead lines.

FIG. 13 shows the injection sites for the intramuscular administration of modified BoNT/A when treating only lateral canthal lines.

FIG. 14 shows the injection sites for the intramuscular administration of modified BoNT/A when treating glabellar lines, forehead lines and lateral canthal lines.

### SEQUENCE LISTING

Where an initial Met amino acid residue or a corresponding initial codon is indicated in any of the following SEQ ID NOs, said residue/codon is optional.

SEQ ID NO: 1 (Nucleotide Sequence of Unmodified BoNT/A)

ATGCCATTCGTCAACAAGCAATTCAACTACAAAGACCCAGTCAACGGCGT
CGACATCGCATACATCAAGATTCCGAACGCCGGTCAAATGCAGCCGGTTA
AGGCTTTTAAGATCCACAACAAGATTTGGGTTATCCCGGAGCGTGACACC
TTCACGAACCCGGAAGAAGGCGATCTGAACCCGCCACCGGAAGCGAAGCA
AGTCCCTGTCAGCTACTACGATTCGACGTACCTGAGCACGGATAACGAAA
AAGATAACTACCTGAAAGGTGTGACCAAGCTGTTCGAACGTATCTACAGC
ACGGATCTGGGTCGCATGCTGCTGACTAGCATTGTTCGCGGTATCCCGTT
CTGGGGTGGTAGCACGATTGACACCGAACTGAAGGTTATCGACACTAACT
GCATTAACGTTATTCAACCGGATGGTAGCTATCGTAGCGAAGAGCTGAAT
CTGGTCATCATTGGCCCGAGCGCAGACATTATCCAATTCGAGTGCAAGAG
CTTTGGTCACGAGGTTCTGAATCTGACCCGCAATGGCTATGGTAGCACCC
AGTACATTCGTTTTTCGCCGGATTTTACCTTCGGCTTTGAAGAGAGCCTG
GAGGTTGATACCAATCCGTTGCTGGGTGCGGGCAAATTCGCTACCGATCC
GGCTGTCACGCTGGCCCATGAACTGATCCACGCAGGCCACCGCCTGTACG
GCATTGCCATCAACCCAAACCGTGTGTTCAAGGTTAATACGAATGCATAC
TACGAGATGAGCGGCCTGGAAGTCAGCTTCGAAGAACTGCGCACCTTCGG
TGGCCATGACGCTAAATTCATTGACAGCTTGCAAGAGAATGAGTTCCGTC
TGTACTACTATAACAAATTCAAAGACATTGCAAGCACGTTGAACAAGGCC
AAAAGCATCGTTGGTACTACCGCGTCGTTGCAGTATATGAAGAATGTGTT
TAAAGAGAAGTACCTGCTGTCCGAGGATACCTCCGGCAAGTTTAGCGTTG
ATAAGCTGAAGTTTGACAAACTGTACAAGATGCTGACCGAGATTTACACC
GAGGACAACTTTGTGAAATTCTTCAAAGTGTTGAATCGTAAAACCTATCT
GAATTTTGACAAAGCGGTTTTCAAGATTAACATCGTGCCGAAGGTGAACT
ACACCATCTATGACGGTTTTAACCTGCGTAACACCAACCTGGCGGCGAAC
TTTAACGGTCAGAATACGGAAATCAACAACATGAATTTCACGAAGTTGAA
GAACTTCACGGGTCTGTTCGAGTTCTATAAGCTGCTGTGCGTGCGCGGTA
TCATCACCAGCAAAACCAAAAGCCTGGACAAAGGCTACAACAAGGCGCTG
AATGACCTGTGCATTAAGGTAAACAATTGGGATCTGTTCTTTTCGCCATC
CGAAGATAATTTTACCAACGACCTGAACAAGGGTGAAGAAATCACCAGCG
ATACGAATATTGAAGCAGCGGAAGAGAATATCAGCCTGGATCTGATCCAG
CAGTACTATCTGACCTTTAACTTCGACAATGAACCGGAGAACATTAGCAT
TGAGAATCTGAGCAGCGACATTATCGGTCAGCTGGAACTGATGCCGAATA
TCGAACGTTTCCCGAACGGCAAAAAGTACGAGCTGGACAAGTACACTATG
TTCCATTACCTGCGTGCACAGGAGTTTGAACACGGTAAAAGCCGTATCGC
GCTGACCAACAGCGTTAACGAGGCCCTGCTGAACCCGAGCCGTGTCTATA
CCTTCTTCAGCAGCGACTATGTTAAGAAAGTGAACAAAGCCACTGAGGCC
GCGATGTTCCTGGGCTGGGTGGAACAGCTGGTATATGACTTCACGGACGA
GACGAGCGAAGTGAGCACTACCGACAAAATTGCTGATATTACCATCATTA
TCCCGTATATTGGTCCGGCACTGAACATTGGCAACATGCTGTACAAAGAC
GATTTTGTGGGTGCCCTGATCTTCTCCGGTGCCGTGATTCTGCTGGAGTT
CATTCCGGAGATTGCGATCCCGGTGTTGGGTACCTTCGCGCTGGTGTCCT
ACATCGCGAATAAGGTTCTGACGGTTCAGACCATCGATAACGCGCTGTCG
AAACGTAATGAAAAATGGGACGAGGTTTACAAATACATTGTTACGAATTG
GCTGGCGAAAGTCAATACCCAGATCGACCTGATCCGTAAGAAAATGAAAG
AGGCGCTGGAGAATCAGGCGGAGGCCACCAAAGCAATTATCAACTACCAA
TACAACCAGTACACGGAAGAAGAGAAGAATAACATTAACTTCAATATCGA
TGATTTGAGCAGCAAGCTGAATGAATCTATCAACAAAGCGATGATCAATA
TCAACAAGTTTTTGAATCAGTGTAGCGTTTCGTACCTGATGAATAGCATG
ATTCCGTATGGCGTCAAACGTCTGGAGGACTTCGACGCCAGCCTGAAAGA
TGCGTTGCTGAAATACATTTACGACAATCGTGGTACGCTGATTGGCCAAG
TTGACCGCTTGAAAGACAAAGTTAACAATACCCTGAGCACCGACATCCCA
TTTCAACTGAGCAAGTATGTTGATAATCAACGTCTGTTGAGCACTTTCAC
CGAGTATATCAAAAACATCATCAATACTAGCATTCTGAACCTGCGTTACG
AGAGCAATCATCTGATTGATCTGAGCCGTTATGCAAGCAAGATCAACATC
GGTAGCAAGGTCAATTTTGACCCGATCGATAAGAACCAGATCCAGCTGTT
TAATCTGGAATCGAGCAAAATTGAGGTTATCCTGAAAAACGCCATTGTCT
ACAACTCCATGTACGAGAATTTCTCCACCAGCTTCTGGATTCGCATCCCG
AAATACTTCAACAGCATTAGCCTGAACAACGAGTATACTATCATCAACTG
TATGGAGAACAACAGCGGTTGGAAGGTGTCTCTGAACTATGGTGAGATCA
TTTGGACCTTGCAGGACACCCAAGAGATCAAGCAGCGCGTCGTGTTCAAG
TACTCTCAAATGATCAACATTTCCGATTACATTAATCGTTGGATCTTCGT
GACCATTACGAATAACCGTCTGAATAACAGCAAGATTTACATCAATGGTC
GCTTGATCGATCAGAAACCGATTAGCAACCTGGGTAATATCCACGCAAGC
AACAACATTATGTTCAAATTGGACGGTTGCCGCGATACCCATCGTTATAT
CTGGATCAAGTATTTCAACCTGTTTGATAAAGAACTGAATGAGAAGGAGA
TCAAAGATTTGTATGACAACCAATCTAACAGCGGCATTTTGAAGGACTTC
TGGGGCGATTATCTGCAATACGATAAGCCGTACTATATGCTGAACCTGTA
TGATCCGAACAAATATGTGGATGTCAATAATGTGGGTATTCGTGGTTACA
TGTATTTGAAGGGTCCGCGTGGCAGCGTTATGACGACCAACATTTACCTG
AACTCTAGCCTGTACCGTGGTACGAAATTCATCATTAAGAAATATGCCAG
CGGCAACAAAGATAACATTGTGCGTAATAACGATCGTGTCTACATCAACG
TGGTCGTGAAGAATAAAGAGTACCGTCTGGCGACCAACGCTTCGCAGGCG
GGTGTTGAGAAAATTCTGAGCGCGTTGGAGATCCCTGATGTCGGTAATCT
GAGCCAAGTCGTGGTTATGAAGAGCAAGAACGACCAGGGTATCACTAACA
AGTGCAAGATGAACCTGCAAGACAACAATGGTAACGACATCGGCTTTATT
GGTTTCCACCAGTTCAACAATATTGCTAAACTGGTAGCGAGCAATTGGTA
CAATCGTCAGATTGAGCGCAGCAGCCGTACTTTGGGCTGTAGCTGGGAGT
TTATCCCGGTCGATGATGGTTGGGGCGAACGTCCGCTG

SEQ ID NO: 2 (Polypeptide Sequence of Unmodified BoNT/A)

MPFVNKQFNYKDPVNGVDIAYIKIPNAGQMQPVKAFKIHNKIWVIPERDT
FTNPEEGDLNPPPEAKQVPVSYYDSTYLSTDNEKDNYLKGVTKLFERIYS
TDLGRMLLTSIVRGIPFWGGSTIDTELKVIDTNCINVIQPDGSYRSEELN
LVIIGPSADIIQFECKSFGHEVLNLTRNGYGSTQYIRFSPDFTFGFEESL
EVDTNPLLGAGKFATDPAVTLAHELIHAGHRLYGIAINPNRVFKVNTNAY
YEMSGLEVSFEELRTFGGHDAKFIDSLQENEFRLYYYNKFKDIASTLNKA
KSIVGTTASLQYMKNVFKEKYLLSEDTSGKFSVDKLKFDKLYKMLTEIYT
EDNFVKFFKVLNRKTYLNFDKAVFKINIVPKVNYTIYDGFNLRNTNLAAN
FNGQNTEINNMNFTKLKNFTGLFEFYKLLCVRGIITSKTKSLDKGYNKAL
NDLCIKVNNWDLFFSPSEDNFTNDLNKGEEITSDTNIEAAEENISLDLIQ
QYYLTFNFDNEPENISIENLSSDIIGQLELMPNIERFPNGKKYELDKYTM
FHYLRAQEFEHGKSRIALTNSVNEALLNPSRVYTFFSSDYVKKVNKATEA
AMFLGWVEQLVYDFTDETSEVSTTDKIADITIIIPYIGPALNIGNMLYKD
DFVGALIFSGAVILLEFIPEIAIPVLGTFALVSYIANKVLTVQTIDNALS
KRNEKWDEVYKYIVTNWLAKVNTQIDLIRKKMKEALENQAEATKAIINYQ
YNQYTEEEKNNINFNIDDLSSKLNESINKAMININKFLNQCSVSYLMNSM
IPYGVKRLEDFDASLKDALLKYIYDNRGTLIGQVDRLKDKVNNTLSTDIP
FQLSKYVDNQRLLSTFTEYIKNIINTSILNLRYESNHLIDLSRYASKINI
GSKVNFDPIDKNQIQLFNLESSKIEVILKNAIVYNSMYENFSTSFWIRIP
KYFNSISLNNEYTIINCMENNSGWKVSLNYGEIIWTLQDTQEIKQRVVFK
YSQMINISDYINRWIFVTITNNRLNNSKIYINGRLIDQKPISNLGNIHAS
NNIMFKLDGCRDTHRYIWIKYFNLFDKELNEKEIKDLYDNQSNSGILKDF
WGDYLQYDKPYYMLNLYDPNKYVDVNNVGIRGYMYLKGPRGSVMTTNIYL
NSSLYRGTKFIIKKYASGNKDNIVRNNDRVYINVVVKNKEYRLATNASQA
GVEKILSALEIPDVGNLSQVVVMKSKNDQGITNKCKMNLQDNNGNDIGFI
GFHQFNNIAKLVASNWYNRQIERSSRTLGCSWEFIPVDDGWGERPL

SEQ ID NO: 3 (Nucleotide Sequence of Modified BoNT/A “Cat-A”)

ATGCCATTCGTCAACAAGCAATTCAACTACAAAGACCCAGTCAACGGCGT
CGACATCGCATACATCAAGATTCCGAACGCCGGTCAAATGCAGCCGGTTA
AGGCTTTTAAGATCCACAACAAGATTTGGGTTATCCCGGAGCGTGACACC
TTCACGAACCCGGAAGAAGGCGATCTGAACCCGCCACCGGAAGCGAAGCA
AGTCCCTGTCAGCTACTACGATTCGACGTACCTGAGCACGGATAACGAAA
AAGATAACTACCTGAAAGGTGTGACCAAGCTGTTCGAACGTATCTACAGC
ACGGATCTGGGTCGCATGCTGCTGACTAGCATTGTTCGCGGTATCCCGTT
CTGGGGTGGTAGCACGATTGACACCGAACTGAAGGTTATCGACACTAACT
GCATTAACGTTATTCAACCGGATGGTAGCTATCGTAGCGAAGAGCTGAAT
CTGGTCATCATTGGCCCGAGCGCAGACATTATCCAATTCGAGTGCAAGAG
CTTTGGTCACGAGGTTCTGAATCTGACCCGCAATGGCTATGGTAGCACCC
AGTACATTCGTTTTTCGCCGGATTTTACCTTCGGCTTTGAAGAGAGCCTG
GAGGTTGATACCAATCCGTTGCTGGGTGCGGGCAAATTCGCTACCGATCC
GGCTGTCACGCTGGCCCATGAACTGATCCACGCAGGCCACCGCCTGTACG
GCATTGCCATCAACCCAAACCGTGTGTTCAAGGTTAATACGAATGCATAC
TACGAGATGAGCGGCCTGGAAGTCAGCTTCGAAGAACTGCGCACCTTCGG
TGGCCATGACGCTAAATTCATTGACAGCTTGCAAGAGAATGAGTTCCGTC
TGTACTACTATAACAAATTCAAAGACATTGCAAGCACGTTGAACAAGGCC
AAAAGCATCGTTGGTACTACCGCGTCGTTGCAGTATATGAAGAATGTGTT
TAAAGAGAAGTACCTGCTGTCCGAGGATACCTCCGGCAAGTTTAGCGTTG
ATAAGCTGAAGTTTGACAAACTGTACAAGATGCTGACCGAGATTTACACC
GAGGACAACTTTGTGAAATTCTTCAAAGTGTTGAATCGTAAAACCTATCT
GAATTTTGACAAAGCGGTTTTCAAGATTAACATCGTGCCGAAGGTGAACT
ACACCATCTATGACGGTTTTAACCTGCGTAACACCAACCTGGCGGCGAAC
TTTAACGGTCAGAATACGGAAATCAACAACATGAATTTCACGAAGTTGAA
GAACTTCACGGGTCTGTTCGAGTTCTATAAGCTGCTGTGCGTGCGCGGTA
TCATCACCAGCAAAACCAAAAGCCTGGACAAAGGCTACAACAAGGCGCTG
AATGACCTGTGCATTAAGGTAAACAATTGGGATCTGTTCTTTTCGCCATC
CGAAGATAATTTTACCAACGACCTGAACAAGGGTGAAGAAATCACCAGCG
ATACGAATATTGAAGCAGCGGAAGAGAATATCAGCCTGGATCTGATCCAG
CAGTACTATCTGACCTTTAACTTCGACAATGAACCGGAGAACATTAGCAT
TGAGAATCTGAGCAGCGACATTATCGGTCAGCTGGAACTGATGCCGAATA
TCGAACGTTTCCCGAACGGCAAAAAGTACGAGCTGGACAAGTACACTATG
TTCCATTACCTGCGTGCACAGGAGTTTGAACACGGTAAAAGCCGTATCGC
GCTGACCAACAGCGTTAACGAGGCCCTGCTGAACCCGAGCCGTGTCTATA
CCTTCTTCAGCAGCGACTATGTTAAGAAAGTGAACAAAGCCACTGAGGCC
GCGATGTTCCTGGGCTGGGTGGAACAGCTGGTATATGACTTCACGGACGA
GACGAGCGAAGTGAGCACTACCGACAAAATTGCTGATATTACCATCATTA
TCCCGTATATTGGTCCGGCACTGAACATTGGCAACATGCTGTACAAAGAC
GATTTTGTGGGTGCCCTGATCTTCTCCGGTGCCGTGATTCTGCTGGAGTT
CATTCCGGAGATTGCGATCCCGGTGTTGGGTACCTTCGCGCTGGTGTCCT
ACATCGCGAATAAGGTTCTGACGGTTCAGACCATCGATAACGCGCTGTCG
AAACGTAATGAAAAATGGGACGAGGTTTACAAATACATTGTTACGAATTG
GCTGGCGAAAGTCAATACCCAGATCGACCTGATCCGTAAGAAAATGAAAG
AGGCGCTGGAGAATCAGGCGGAGGCCACCAAAGCAATTATCAACTACCAA
TACAACCAGTACACGGAAGAAGAGAAGAATAACATTAACTTCAATATCGA
TGATTTGAGCAGCAAGCTGAATGAATCTATCAACAAAGCGATGATCAATA
TCAACAAGTTTTTGAATCAGTGTAGCGTTTCGTACCTGATGAATAGCATG
ATTCCGTATGGCGTCAAACGTCTGGAGGACTTCGACGCCAGCCTGAAAGA
TGCGTTGCTGAAATACATTTACGACAATCGTGGTACGCTGATTGGCCAAG
TTGACCGCTTGAAAGACAAAGTTAACAATACCCTGAGCACCGACATCCCA
TTTCAACTGAGCAAGTATGTTGATAATCAACGTCTGTTGAGCACTTTCAC
CGAGTATATCAAAAACATCATCAATACTAGCATTCTGAACCTGCGTTACG
AGAGCAAGCATCTGATTGATCTGAGCCGTTATGCTAGCAAGATCAACATC
GGTAGCAAGGTCAATTTTGACCCGATCGATAAGAACCAGATCCAGCTGTT
TAATCTGGAATCGAGCAAAATTGAGGTTATCCTGAAAAAGGCCATTGTCT
ACAACTCCATGTACGAGAATTTCTCCACCAGCTTCTGGATTCGCATCCCG
AAATACTTCAACAAGATTAGCCTGAACAACGAGTATACTATCATCAACTG
TATGGAGAACAACAGCGGTTGGAAGGTGTCTCTGAACTATGGTGAGATCA
TTTGGACCTTGCAGGACACCAAAGAGATCAAGCAGCGCGTCGTGTTCAAG
TACTCTCAAATGATCAACATTTCCGATTACATTAATCGTTGGATCTTCGT
GACCATTACGAATAACCGTCTGAATAAGAGCAAGATTTACATCAATGGTC
GCTTGATCGATCAGAAACCGATTAGCAACCTGGGTAATATCCACGCAAGC
AACAAGATTATGTTCAAATTGGACGGTTGCCGCGATACCCATCGTTATAT
CTGGATCAAGTATTTCAACCTGTTTGATAAAGAACTGAATGAGAAGGAGA
TCAAAGATTTGTATGACAACCAATCTAACAGCGGCATTTTGAAGGACTTC
TGGGGCGATTATCTGCAATACGATAAGCCGTACTATATGCTGAACCTGTA
TGATCCGAACAAATATGTGGATGTCAATAATGTGGGTATTCGTGGTTACA
TGTATTTGAAGGGTCCGCGTGGCAGCGTTATGACGACCAACATTTACCTG
AACTCTAGCCTGTACCGTGGTACGAAATTCATCATTAAGAAATATGCCAG
CGGCAACAAAGATAACATTGTGCGTAATAACGATCGTGTCTACATCAACG
TGGTCGTGAAGAATAAAGAGTACCGTCTGGCGACCAACGCTTCGCAGGCG
GGTGTTGAGAAAATTCTGAGCGCGTTGGAGATCCCTGATGTCGGTAATCT
GAGCCAAGTCGTGGTTATGAAGAGCAAGAACGACAAGGGTATCACTAACA
AGTGCAAGATGAACCTGCAAGACAACAATGGTAACGACATCGGCTTTATT
GGTTTCCACCAGTTCAACAATATTGCTAAACTGGTAGCGAGCAATTGGTA
CAATCGTCAGATTGAGCGCAGCAGCcGTACTTTGGGCTGTAGCTGGGAGT
TTATCCCGGTCGATGATGGTTGGGGCGAACGTCCGCTG

SEQ ID NO: 4 (Polypeptide Sequence of Modified BoNT/A “Cat-A”)

MPFVNKQFNYKDPVNGVDIAYIKIPNAGQMQPVKAFKIHNKIWVIPERDT
FTNPEEGDLNPPPEAKQVPVSYYDSTYLSTDNEKDNYLKGVTKLFERIYS
TDLGRMLLTSIVRGIPFWGGSTIDTELKVIDTNCINVIQPDGSYRSEELN
LVIIGPSADIIQFECKSFGHEVLNLTRNGYGSTQYIRFSPDFTFGFEESL
EVDTNPLLGAGKFATDPAVTLAHELIHAGHRLYGIAINPNRVFKVNTNAY
YEMSGLEVSFEELRTFGGHDAKFIDSLQENEFRLYYYNKFKDIASTLNKA
KSIVGTTASLQYMKNVFKEKYLLSEDTSGKFSVDKLKFDKLYKMLTEIYT
EDNFVKFFKVLNRKTYLNFDKAVFKINIVPKVNYTIYDGFNLRNTNLAAN
FNGQNTEINNMNFTKLKNFTGLFEFYKLLCVRGIITSKTKSLDKGYNKAL
NDLCIKVNNWDLFFSPSEDNFTNDLNKGEEITSDTNIEAAEENISLDLIQ
QYYLTFNFDNEPENISIENLSSDIIGQLELMPNIERFPNGKKYELDKYTM
FHYLRAQEFEHGKSRIALTNSVNEALLNPSRVYTFFSSDYVKKVNKATEA
AMFLGWVEQLVYDFTDETSEVSTTDKIADITIIIPYIGPALNIGNMLYKD
DFVGALIFSGAVILLEFIPEIAIPVLGTFALVSYIANKVLTVQTIDNALS
KRNEKWDEVYKYIVTNWLAKVNTQIDLIRKKMKEALENQAEATKAIINYQ
YNQYTEEEKNNINFNIDDLSSKLNESINKAMININKFLNQCSVSYLMNSM
IPYGVKRLEDFDASLKDALLKYIYDNRGTLIGQVDRLKDKVNNTLSTDIP
FQLSKYVDNQRLLSTFTEYIKNIINTSILNLRYESKHLIDLSRYASKINI
GSKVNFDPIDKNQIQLFNLESSKIEVILKKAIVYNSMYENFSTSFWIRIP
KYFNKISLNNEYTIINCMENNSGWKVSLNYGEIIWTLQDTKEIKQRVVFK
YSQMINISDYINRWIFVTITNNRLNKSKIYINGRLIDQKPISNLGNIHAS
NKIMFKLDGCRDTHRYIWIKYFNLFDKELNEKEIKDLYDNQSNSGILKDF
WGDYLQYDKPYYMLNLYDPNKYVDVNNVGIRGYMYLKGPRGSVMTTNIYL
NSSLYRGTKFIIKKYASGNKDNIVRNNDRVYINVVVKNKEYRLATNASQA
GVEKILSALEIPDVGNLSQVVVMKSKNDKGITNKCKMNLQDNNGNDIGFI
GFHQFNNIAKLVASNWYNRQIERSSRTLGCSWEFIPVDDGWGERPL

SEQ ID NO: 5 (Nucleotide Sequence of Modified BoNT/A “Cat-B″)

ATGCCATTCGTCAACAAGCAATTCAACTACAAAGACCCAGTCAACGGCGT
CGACATCGCATACATCAAGATTCCGAACGCCGGTCAAATGCAGCCGGTTA
AGGCTTTTAAGATCCACAACAAGATTTGGGTTATCCCGGAGCGTGACACC
TTCACGAACCCGGAAGAAGGCGATCTGAACCCGCCACCGGAAGCGAAGCA
AGTCCCTGTCAGCTACTACGATTCGACGTACCTGAGCACGGATAACGAAA
AAGATAACTACCTGAAAGGTGTGACCAAGCTGTTCGAACGTATCTACAGC
ACGGATCTGGGTCGCATGCTGCTGACTAGCATTGTTCGCGGTATCCCGTT
CTGGGGTGGTAGCACGATTGACACCGAACTGAAGGTTATCGACACTAACT
GCATTAACGTTATTCAACCGGATGGTAGCTATCGTAGCGAAGAGCTGAAT
CTGGTCATCATTGGCCCGAGCGCAGACATTATCCAATTCGAGTGCAAGAG
CTTTGGTCACGAGGTTCTGAATCTGACCCGCAATGGCTATGGTAGCACCC
AGTACATTCGTTTTTCGCCGGATTTTACCTTCGGCTTTGAAGAGAGCCTG
GAGGTTGATACCAATCCGTTGCTGGGTGCGGGCAAATTCGCTACCGATCC
GGCTGTCACGCTGGCCCATGAACTGATCCACGCAGGCCACCGCCTGTACG
GCATTGCCATCAACCCAAACCGTGTGTTCAAGGTTAATACGAATGCATAC
TACGAGATGAGCGGCCTGGAAGTCAGCTTCGAAGAACTGCGCACCTTCGG
TGGCCATGACGCTAAATTCATTGACAGCTTGCAAGAGAATGAGTTCCGTC
TGTACTACTATAACAAATTCAAAGACATTGCAAGCACGTTGAACAAGGCC
AAAAGCATCGTTGGTACTACCGCGTCGTTGCAGTATATGAAGAATGTGTT
TAAAGAGAAGTACCTGCTGTCCGAGGATACCTCCGGCAAGTTTAGCGTTG
ATAAGCTGAAGTTTGACAAACTGTACAAGATGCTGACCGAGATTTACACC
GAGGACAACTTTGTGAAATTCTTCAAAGTGTTGAATCGTAAAACCTATCT
GAATTTTGACAAAGCGGTTTTCAAGATTAACATCGTGCCGAAGGTGAACT
ACACCATCTATGACGGTTTTAACCTGCGTAACACCAACCTGGCGGCGAAC
TTTAACGGTCAGAATACGGAAATCAACAACATGAATTTCACGAAGTTGAA
GAACTTCACGGGTCTGTTCGAGTTCTATAAGCTGCTGTGCGTGCGCGGTA
TCATCACCAGCAAAACCAAAAGCCTGGACAAAGGCTACAACAAGGCGCTG
AATGACCTGTGCATTAAGGTAAACAATTGGGATCTGTTCTTTTCGCCATC
CGAAGATAATTTTACCAACGACCTGAACAAGGGTGAAGAAATCACCAGCG
ATACGAATATTGAAGCAGCGGAAGAGAATATCAGCCTGGATCTGATCCAG
CAGTACTATCTGACCTTTAACTTCGACAATGAACCGGAGAACATTAGCAT
TGAGAATCTGAGCAGCGACATTATCGGTCAGCTGGAACTGATGCCGAATA
TCGAACGTTTCCCGAACGGCAAAAAGTACGAGCTGGACAAGTACACTATG
TTCCATTACCTGCGTGCACAGGAGTTTGAACACGGTAAAAGCCGTATCGC
GCTGACCAACAGCGTTAACGAGGCCCTGCTGAACCCGAGCCGTGTCTATA
CCTTCTTCAGCAGCGACTATGTTAAGAAAGTGAACAAAGCCACTGAGGCC
GCGATGTTCCTGGGCTGGGTGGAACAGCTGGTATATGACTTCACGGACGA
GACGAGCGAAGTGAGCACTACCGACAAAATTGCTGATATTACCATCATTA
TCCCGTATATTGGTCCGGCACTGAACATTGGCAACATGCTGTACAAAGAC
GATTTTGTGGGTGCCCTGATCTTCTCCGGTGCCGTGATTCTGCTGGAGTT
CATTCCGGAGATTGCGATCCCGGTGTTGGGTACCTTCGCGCTGGTGTCCT
ACATCGCGAATAAGGTTCTGACGGTTCAGACCATCGATAACGCGCTGTCG
AAACGTAATGAAAAATGGGACGAGGTTTACAAATACATTGTTACGAATTG
GCTGGCGAAAGTCAATACCCAGATCGACCTGATCCGTAAGAAAATGAAAG
AGGCGCTGGAGAATCAGGCGGAGGCCACCAAAGCAATTATCAACTACCAA
TACAACCAGTACACGGAAGAAGAGAAGAATAACATTAACTTCAATATCGA
TGATTTGAGCAGCAAGCTGAATGAATCTATCAACAAAGCGATGATCAATA
TCAACAAGTTTTTGAATCAGTGTAGCGTTTCGTACCTGATGAATAGCATG
ATTCCGTATGGCGTCAAACGTCTGGAGGACTTCGACGCCAGCCTGAAAGA
TGCGTTGCTGAAATACATTTACGACAaTCGTGGTACGCTGATTGGCCAAG
TTGACCGCTTGAAAGACAAAGTTAACAATACCCTGAGCACCGACATCCCA
TTTCAACTGAGCAAGTATGTTGATAATCAACGTCTGTTGAGCACTTTCAC
CGAGTATATCAAAAACATCATCAATACTAGCATTCTGAACCTGCGTTACG
AGAGCAATCATCTGATTGATCTGAGCCGTTATGCTAGCAAGATCAACATC
GGTAGCAAGGTCAATTTTGACCCGATCGATAAGAACCAGATCCAGCTGTT
TAATCTGGAATCGAGCAAAATTGAGGTTATCCTGAAAAAGGCCATTGTCT
ACAACTCCATGTACGAGAATTTCTCCACCAGCTTCTGGATTCGCATCCCG
AAATACTTCAAGAAGATTAGCCTGAACAACGAGTATACTATCATCAACTG
TATGGAGAACAACAGCGGTTGGAAGGTGTCTCTGAACTATGGTGAGATCA
TTTGGACCTTGCAGGACACCAAAGAGATCAAGCAGCGCGTCGTGTTCAAG
TACTCTCAAATGATCAACATTTCCGATTACATTAATCGTTGGATCTTCGT
GACCATTACGAATAACCGTCTGAATAAGAGCAAGATTTACATCAATGGTC
GCTTGATCGATCAGAAACCGATTAGCAACCTGGGTAATATCCACGCAAGC
AACAAGATTATGTTCAAATTGGACGGTTGCCGCGATACCCATCGTTATAT
CTGGATCAAGTATTTCAACCTGTTTGATAAAGAACTGAATGAGAAGGAGA
TCAAAGATTTGTATGACAACCAATCTAACAGCGGCATTTTGAAGGACTTC
TGGGGCGATTATCTGCAATACGATAAGCCGTACTATATGCTGAACCTGTA
TGATCCGAACAAATATGTGGATGTCAATAATGTGGGTATTCGTGGTTACA
TGTATTTGAAGGGTCCGCGTGGCAGCGTTATGACGACCAACATTTACCTG
AACTCTAGCCTGTACCGTGGTACGAAATTCATCATTAAGAAATATGCCAG
CGGCAACAAAGATAACATTGTGCGTAATAACGATCGTGTCTACATCAACG
TGGTCGTGAAGAATAAAGAGTACCGTCTGGCGACCAACGCTTCGCAGGCG
GGTGTTGAGAAAATTCTGAGCGCGTTGGAGATCCCTGATGTCGGTAATCT
GAGCCAAGTCGTGGTTATGAAGAGCAAGAACGACAAGGGTATCACTAACA
AGTGCAAGATGAACCTGCAAGACAACAATGGTAACGACATCGGCTTTATT
GGTTTCCACCAGTTCAACAATATTGCTAAACTGGTAGCGAGCAATTGGTA
CAATCGTCAGATTGAGCGCAGCAGCCGTACTTTGGGCTGTAGCTGGGAGT
TTATCCCGGTCGATGATGGTTGGGGCGAACGTCCGCTG

SEQ ID NO: 6 (Polypeptide Sequence of Modified BoNT/A “Cat-B″)

MPFVNKQFNYKDPVNGVDIAYIKIPNAGQMQPVKAFKIHNKIWVIPERDT
FTNPEEGDLNPPPEAKQVPVSYYDSTYLSTDNEKDNYLKGVTKLFERIYS
TDLGRMLLTSIVRGIPFWGGSTIDTELKVIDTNCINVIQPDGSYRSEELN
LVIIGPSADIIQFECKSFGHEVLNLTRNGYGSTQYIRFSPDFTFGFEESL
EVDTNPLLGAGKFATDPAVTLAHELIHAGHRLYGIAINPNRVFKVNTNAY
YEMSGLEVSFEELRTFGGHDAKFIDSLQENEFRLYYYNKFKDIASTLNKA
KSIVGTTASLQYMKNVFKEKYLLSEDTSGKFSVDKLKFDKLYKMLTEIYT
EDNFVKFFKVLNRKTYLNFDKAVFKINIVPKVNYTIYDGFNLRNTNLAAN
FNGQNTEINNMNFTKLKNFTGLFEFYKLLCVRGIITSKTKSLDKGYNKAL
NDLCIKVNNWDLFFSPSEDNFTNDLNKGEEITSDTNIEAAEENISLDLIQ
QYYLTFNFDNEPENISIENLSSDIIGQLELMPNIERFPNGKKYELDKYTM
FHYLRAQEFEHGKSRIALTNSVNEALLNPSRVYTFFSSDYVKKVNKATEA
AMFLGWVEQLVYDFTDETSEVSTTDKIADITIIIPYIGPALNIGNMLYKD
DFVGALIFSGAVILLEFIPEIAIPVLGTFALVSYIANKVLTVQTIDNALS
KRNEKWDEVYKYIVTNWLAKVNTQIDLIRKKMKEALENQAEATKAIINYQ
YNQYTEEEKNNINFNIDDLSSKLNESINKAMININKFLNQCSVSYLMNSM
IPYGVKRLEDFDASLKDALLKYIYDNRGTLIGQVDRLKDKVNNTLSTDIP
FQLSKYVDNQRLLSTFTEYIKNIINTSILNLRYESNHLIDLSRYASKINI
GSKVNFDPIDKNQIQLFNLESSKIEVILKKAIVYNSMYENFSTSFWIRIP
KYFKKISLNNEYTIINCMENNSGWKVSLNYGEIIWTLQDTKEIKQRVVFK
YSQMINISDYINRWIFVTITNNRLNKSKIYINGRLIDQKPISNLGNIHAS
NKIMFKLDGCRDTHRYIWIKYFNLFDKELNEKEIKDLYDNQSNSGILKDF
WGDYLQYDKPYYMLNLYDPNKYVDVNNVGIRGYMYLKGPRGSVMTTNIYL
NSSLYRGTKFIIKKYASGNKDNIVRNNDRVYINVVVKNKEYRLATNASQA
GVEKILSALEIPDVGNLSQVVVMKSKNDKGITNKCKMNLQDNNGNDIGFI
GFHQFNNIAKLVASNWYNRQIERSSRTLGCSWEFIPVDDGWGERPL

SEQ ID NO: 7 (Nucleotide Sequence of Modified BoNT/A “Cat-C″)

ATGCCATTCGTCAACAAGCAATTCAACTACAAAGACCCAGTCAACGGCGT
CGACATCGCATACATCAAGATTCCGAACGCCGGTCAAATGCAGCCGGTTA
AGGCTTTTAAGATCCACAACAAGATTTGGGTTATCCCGGAGCGTGACACC
TTCACGAACCCGGAAGAAGGCGATCTGAACCCGCCACCGGAAGCGAAGCA
AGTCCCTGTCAGCTACTACGATTCGACGTACCTGAGCACGGATAACGAAA
AAGATAACTACCTGAAAGGTGTGACCAAGCTGTTCGAACGTATCTACAGC
ACGGATCTGGGTCGCATGCTGCTGACTAGCATTGTTCGCGGTATCCCGTT
CTGGGGTGGTAGCACGATTGACACCGAACTGAAGGTTATCGACACTAACT
GCATTAACGTTATTCAACCGGATGGTAGCTATCGTAGCGAAGAGCTGAAT
CTGGTCATCATTGGCCCGAGCGCAGACATTATCCAATTCGAGTGCAAGAG
CTTTGGTCACGAGGTTCTGAATCTGACCCGCAATGGCTATGGTAGCACCC
AGTACATTCGTTTTTCGCCGGATTTTACCTTCGGCTTTGAAGAGAGCCTG
GAGGTTGATACCAATCCGTTGCTGGGTGCGGGCAAATTCGCTACCGATCC
GGCTGTCACGCTGGCCCATGAACTGATCCACGCAGGCCACCGCCTGTACG
GCATTGCCATCAACCCAAACCGTGTGTTCAAGGTTAATACGAATGCATAC
TACGAGATGAGCGGCCTGGAAGTCAGCTTCGAAGAACTGCGCACCTTCGG
TGGCCATGACGCTAAATTCATTGACAGCTTGCAAGAGAATGAGTTCCGTC
TGTACTACTATAACAAATTCAAAGACATTGCAAGCACGTTGAACAAGGCC
AAAAGCATCGTTGGTACTACCGCGTCGTTGCAGTATATGAAGAATGTGTT
TAAAGAGAAGTACCTGCTGTCCGAGGATACCTCCGGCAAGTTTAGCGTTG
ATAAGCTGAAGTTTGACAAACTGTACAAGATGCTGACCGAGATTTACACC
GAGGACAACTTTGTGAAATTCTTCAAAGTGTTGAATCGTAAAACCTATCT
GAATTTTGACAAAGCGGTTTTCAAGATTAACATCGTGCCGAAGGTGAACT
ACACCATCTATGACGGTTTTAACCTGCGTAACACCAACCTGGCGGCGAAC
TTTAACGGTCAGAATACGGAAATCAACAACATGAATTTCACGAAGTTGAA
GAACTTCACGGGTCTGTTCGAGTTCTATAAGCTGCTGTGCGTGCGCGGTA
TCATCACCAGCAAAACCAAAAGCCTGGACAAAGGCTACAACAAGGCGCTG
AATGACCTGTGCATTAAGGTAAACAATTGGGATCTGTTCTTTTCGCCATC
CGAAGATAATTTTACCAACGACCTGAACAAGGGTGAAGAAATCACCAGCG
ATACGAATATTGAAGCAGCGGAAGAGAATATCAGCCTGGATCTGATCCAG
CAGTACTATCTGACCTTTAACTTCGACAATGAACCGGAGAACATTAGCAT
TGAGAATCTGAGCAGCGACATTATCGGTCAGCTGGAACTGATGCCGAATA
TCGAACGTTTCCCGAACGGCAAAAAGTACGAGCTGGACAAGTACACTATG
TTCCATTACCTGCGTGCACAGGAGTTTGAACACGGTAAAAGCCGTATCGC
GCTGACCAACAGCGTTAACGAGGCCCTGCTGAACCCGAGCCGTGTCTATA
CCTTCTTCAGCAGCGACTATGTTAAGAAAGTGAACAAAGCCACTGAGGCC
GCGATGTTCCTGGGCTGGGTGGAACAGCTGGTATATGACTTCACGGACGA
GACGAGCGAAGTGAGCACTACCGACAAAATTGCTGATATTACCATCATTA
TCCCGTATATTGGTCCGGCACTGAACATTGGCAACATGCTGTACAAAGAC
GATTTTGTGGGTGCCCTGATCTTCTCCGGTGCCGTGATTCTGCTGGAGTT
CATTCCGGAGATTGCGATCCCGGTGTTGGGTACCTTCGCGCTGGTGTCCT
ACATCGCGAATAAGGTTCTGACGGTTCAGACCATCGATAACGCGCTGTCG
AAACGTAATGAAAAATGGGACGAGGTTTACAAATACATTGTTACGAATTG
GCTGGCGAAAGTCAATACCCAGATCGACCTGATCCGTAAGAAAATGAAAG
AGGCGCTGGAGAATCAGGCGGAGGCCACCAAAGCAATTATCAACTACCAA
TACAACCAGTACACGGAAGAAGAGAAGAATAACATTAACTTCAATATCGA
TGATTTGAGCAGCAAGCTGAATGAATCTATCAACAAAGCGATGATCAATA
TCAACAAGTTTTTGAATCAGTGTAGCGTTTCGTACCTGATGAATAGCATG
ATTCCGTATGGCGTCAAACGTCTGGAGGACTTCGACGCCAGCCTGAAAGA
TGCGTTGCTGAAATACATTTACGACAATCGTGGTACGCTGATTGGCCAAG
TTGACCGCTTGAAAGACAAAGTTAACAATACCCTGAGCACCGACATCCCA
TTTCAACTGAGCAAGTATGTTGATAATCAACGTCTGTTGAGCACTTTCAC
CGAGTATATCAAAAACATCATCAATACTAGCATTCTGAACCTGCGTTACG
AGAGCAATCATCTGATTGATCTGAGCCGTTATGCTAGCAAGATCAACATC
GGTAGCAAGGTCAATTTTGACCCGATCGATAAGAACCAGATCCAGCTGTT
TAATCTGGAATCGAGCAAAATTGAGGTTATCCTGAAAAAGGCCATTGTCT
ACAACTCCATGTACGAGAATTTCTCCACCAGCTTCTGGATTCGCATCCCG
AAATACTTCAACAAGATTAGCCTGAACAACGAGTATACTATCATCAACTG
TATGGAGAACAACAGCGGTTGGAAGGTGTCTCTGAACTATGGTGAGATCA
TTTGGACCTTGCAGGACACCAAAGAGATCAAGCAGCGCGTCGTGTTCAAG
TACTCTCAAATGATCAACATTTCCGATTACATTAATCGTTGGATCTTCGT
GACCATTACGAATAACCGTCTGAAGAAGAGCAAGATTTACATCAATGGTC
GCTTGATCGATCAGAAACCGATTAGCAACCTGGGTAATATCCACGCAAGC
AACAAGATTATGTTCAAATTGGACGGTTGCCGCGATACCCATCGTTATAT
CTGGATCAAGTATTTCAACCTGTTTGATAAAGAACTGAATGAGAAGGAGA
TCAAAGATTTGTATGACAACCAATCTAACAGCGGCATTTTGAAGGACTTC
TGGGGCGATTATCTGCAATACGATAAGCCGTACTATATGCTGAACCTGTA
TGATCCGAACAAATATGTGGATGTCAATAATGTGGGTATTCGTGGTTACA
TGTATTTGAAGGGTCCGCGTGGCAGCGTTATGACGACCAACATTTACCTG
AACTCTAGCCTGTACCGTGGTACGAAATTCATCATTAAGAAATATGCCAG
CGGCAACAAAGATAACATTGTGCGTAATAACGATCGTGTCTACATCAACG
TGGTCGTGAAGAATAAAGAGTACCGTCTGGCGACCAACGCTTCGCAGGCG
GGTGTTGAGAAAATTCTGAGCGCGTTGGAGATCCCTGATGTCGGTAATCT
GAGCCAAGTCGTGGTTATGAAGAGCAAGAACGACAAGGGTATCACTAACA
AGTGCAAGATGAACCTGCAAGACAACAATGGTAACGACATCGGCTTTATT
GGTTTCCACCAGTTCAACAATATTGCTAAACTGGTAGCGAGCAATTGGTA
CAATCGTCAGATTGAGCGCAGCAGCCGTACTTTGGGCTGTAGCTGGGAGT
TTATCCCGGTCGATGATGGTTGGGGCGAACGTCCGCTG

SEQ ID NO: 8 (Polypeptide Sequence of Modified BoNT/A “Cat-C″)

MPFVNKQFNYKDPVNGVDIAYIKIPNAGQMQPVKAFKIHNKIWVIPERDT
FTNPEEGDLNPPPEAKQVPVSYYDSTYLSTDNEKDNYLKGVTKLFERIYS
TDLGRMLLTSIVRGIPFWGGSTIDTELKVIDTNCINVIQPDGSYRSEELN
LVIIGPSADIIQFECKSFGHEVLNLTRNGYGSTQYIRFSPDFTFGFEESL
EVDTNPLLGAGKFATDPAVTLAHELIHAGHRLYGIAINPNRVFKVNTNAY
YEMSGLEVSFEELRTFGGHDAKFIDSLQENEFRLYYYNKFKDIASTLNKA
KSIVGTTASLQYMKNVFKEKYLLSEDTSGKFSVDKLKFDKLYKMLTEIYT
EDNFVKFFKVLNRKTYLNFDKAVFKINIVPKVNYTIYDGFNLRNTNLAAN
FNGQNTEINNMNFTKLKNFTGLFEFYKLLCVRGIITSKTKSLDKGYNKAL
NDLCIKVNNWDLFFSPSEDNFTNDLNKGEEITSDTNIEAAEENISLDLIQ
QYYLTFNFDNEPENISIENLSSDIIGQLELMPNIERFPNGKKYELDKYTM
FHYLRAQEFEHGKSRIALTNSVNEALLNPSRVYTFFSSDYVKKVNKATEA
AMFLGWVEQLVYDFTDETSEVSTTDKIADITIIIPYIGPALNIGNMLYKD
DFVGALIFSGAVILLEFIPEIAIPVLGTFALVSYIANKVLTVQTIDNALS
KRNEKWDEVYKYIVTNWLAKVNTQIDLIRKKMKEALENQAEATKAIINYQ
YNQYTEEEKNNINFNIDDLSSKLNESINKAMININKFLNQCSVSYLMNSM
IPYGVKRLEDFDASLKDALLKYIYDNRGTLIGQVDRLKDKVNNTLSTDIP
FQLSKYVDNQRLLSTFTEYIKNIINTSILNLRYESNHLIDLSRYASKINI
GSKVNFDPIDKNQIQLFNLESSKIEVILKKAIVYNSMYENFSTSFWIRIP
KYFNKISLNNEYTIINCMENNSGWKVSLNYGEIIWTLQDTKEIKQRVVFK
YSQMINISDYINRWIFVTITNNRLKKSKIYINGRLIDQKPISNLGNIHAS
NKIMFKLDGCRDTHRYIWIKYFNLFDKELNEKEIKDLYDNQSNSGILKDF
WGDYLQYDKPYYMLNLYDPNKYVDVNNVGIRGYMYLKGPRGSVMTTNIYL
NSSLYRGTKFIIKKYASGNKDNIVRNNDRVYINVVVKNKEYRLATNASQA
GVEKILSALEIPDVGNLSQVVVMKSKNDKGITNKCKMNLQDNNGNDIGFI
GFHQFNNIAKLVASNWYNRQIERSSRTLGCSWEFIPVDDGWGERPL

SEQ ID NO: 9 (Nucleotide Sequence of Modified BoNT/A “Cat-D”)

ATGCCATTCGTCAACAAGCAATTCAACTACAAAGACCCAGTCAACGGCGT
CGACATCGCATACATCAAGATTCCGAACGCCGGTCAAATGCAGCCGGTTA
AGGCTTTTAAGATCCACAACAAGATTTGGGTTATCCCGGAGCGTGACACC
TTCACGAACCCGGAAGAAGGCGATCTGAACCCGCCACCGGAAGCGAAGCA
AGTCCCTGTCAGCTACTACGATTCGACGTACCTGAGCACGGATAACGAAA
AAGATAACTACCTGAAAGGTGTGACCAAGCTGTTCGAACGTATCTACAGC
ACGGATCTGGGTCGCATGCTGCTGACTAGCATTGTTCGCGGTATCCCGTT
CTGGGGTGGTAGCACGATTGACACCGAACTGAAGGTTATCGACACTAACT
GCATTAACGTTATTCAACCGGATGGTAGCTATCGTAGCGAAGAGCTGAAT
CTGGTCATCATTGGCCCGAGCGCAGACATTATCCAATTCGAGTGCAAGAG
CTTTGGTCACGAGGTTCTGAATCTGACCCGCAATGGCTATGGTAGCACCC
AGTACATTCGTTTTTCGCCGGATTTTACCTTCGGCTTTGAAGAGAGCCTG
GAGGTTGATACCAATCCGTTGCTGGGTGCGGGCAAATTCGCTACCGATCC
GGCTGTCACGCTGGCCCATGAACTGATCCACGCAGGCCACCGCCTGTACG
GCATTGCCATCAACCCAAACCGTGTGTTCAAGGTTAATACGAATGCATAC
TACGAGATGAGCGGCCTgGAAGTCAGCTTCGAAGAACTGCGCACCTTCGG
TGGCCATGACGCTAAATTCATTGACAGCTTGCAAGAGAATGAGTTCCGTC
TGTACTACTATAACAAATTCAAAGACATTGCAAGCACGTTGAACAAGGCC
AAAAGCATCGTTGGTACTACCGCGTCGTTGCAGTATATGAAGAATGTGTT
TAAAGAGAAGTACCTGCTGTCCGAGGATACCTCCGGCAAGTTTAGCGTTG
ATAAGCTGAAGTTTGACAAACTGTACAAGATGCTGACCGAGATTTACACC
GAGGACAACTTTGTGAAATTCTTCAAaGTGTTGAATCGTAAAACCTATCT
GAATTTTGACAAAGCGGTTTTCaAGATTAACATCGTGCCGAAGGTGAACT
ACACCATCTATGACGGTTTTAACCTGCGTAACACCAACCTGGCGGCGAAC
TTTAACGGTCAGAATACGGAAATCAACAACATGAATTTCACGAAGTTGAA
GAACTTCACGGGTCTGTTCGAGTTCTATAAGCTGCTGTGCGTGCGCGGTA
TCATCACCAGCAAAACCAAAAGCCTGGACAAAGGCTACAACAAGGCGCTG
AATGACCTGTGCATTAAGGTAAACAATTGGGATCTGTTCTTTTCGCCATC
CGAAGATAATTTTACCAACGACCTGAACAAGGGTGAAGAAATCACCAGCG
ATACGAATATTGAAGCAGCGGAAGAGAATATCAGCCTGGATCTGATCCAG
CAGTACTATCTGACCTTTAACTTCGACAATGAACCGGAGAACATTAGCAT
TGAGAATCTGAGCAGCGACATTATCGGTCAGCTGGAACTGATGCCGAATA
TCGAACGTTTCCCGAACGGCAAAAAGTACGAGCTGGACAAGTACACTATG
TTCCATTACCTGCGTGCACAGGAGTTTGAACACGGTAAAAGCCGTATCGC
GCTGACCAACAGCGTTAACGAGGCCCTGCTGAACCCGAGCCGTGTCTATA
CCTTCTTCAGCAGCGACTATGTTAAGAAAGTGAACAAAGCCACTGAGGCC
GCGATGTTCCTGGGCTGGGTGGAACAGCTGGTATATGACTTCACGGACGA
GACGAGCGAAGTGAGCACTACCGACAAAaTTGCTGATaTTACCATCATTA
TCCCGTATATTGGTCCGGCACTGAACATTGGCAACATGCTGTACAAAGAC
GATTTTGTGGGTGCCCTGATCTTCTCCGGTGCCGTGATTCTGCTGGAGTT
CATTCCGGAGATTGCGATCCCGGTGTTGGGTACCTTCGCGCTGGTGTCCT
ACATCGCGAATAAGGTTCTGACGGTTCAGACCATCGATAACGCGCTGTCG
AAACGTAATGAAAAATGGGACGAGGTTTACAAATACATTGTTACGAATTG
GCTGGCGAAAGTCaATACCCAGATCGACCTGATCCGTAAGAAAATGAAAG
AGGCGCTGGAGAATCAGGCGGAGGCCACCAAAGCAATTATCAACTACCAA
TACAACCAGTACACGGAAGAAGAGAAGAATAACATTAACTTCAATATCGA
TGATTTGAGCAGCAAGCTGAATGAATCTATCAACAAAGCGATGATCAATA
TCAACAAGTTTTTGAATCAGTGTAGCGTTTCGTACCTGATGAATAGCATG
ATTCCGTATGGCGTCAAACGTCTGGAGGACTTCGACGCCAGCCTGAAAGA
TGCGTTGCTGAAATACATTTACGACAATCGTGGTACGCTGATTGGCCAAG
TTGACCGCTTGAAAGACAAAGTTAACAATACCCTGAGCACCGACATCCCA
TTTCAACTGAGCAAGTATGTTGATAATCAACGTCTGTTGAGCACTTTCAC
CGAGTATATCAAAAACATCATCAATACTAGCATTCTGAACCTGCGTTACG
AGAGCAATCATCTGATtGATCTGAGCCGTTATGCAAGCAAGATCAACATC
GGTAGCAAGGTCAATTTTGACCCGATCGATAAGAACCAGATCCAGCTGTT
TAATCTGGAATCGAGCAAAATTGAGGTTATCCTGAAAAACGCCATTGTCT
ACAACTCCATGTACGAGAATTTCTCCACCAGCTTCTGGATTCGCATCCCG
AAATACTTCAACAGCATTAGCCTGAACAACGAGTATACTATCATCAACTG
TATGGAGAACAACAGCGGTTGGAAGGTGTCTCTGAACTATGGTGAGATCA
TTTGGACCTTGCAGGACACCCAAGAGATCAAGCAGCGCGTCGTGTTCAAG
TACTCTCAAATGATCAACATTTCCGATTACATTAATCGTTGGATCTTCGT
GACCATTACGAATAACCGTCTGAATAACAGCAAGATTTACATCAATGGTC
GCTTGATCGATCAGAAACCGATTAGCAACCTGGGTAATATCCACGCAAGC
AACAACATTATGTTCAAATTGGACGGTTGCCGCGATACCCATCGTTATAT
CTGGATCAAGTATTTCAACCTGTTTGATAAAGAACTGAATGAGAAGGAGA
TCAAAGATTTGTATGACAACCAATCTAACAGCGGCATTTTGAAGGACTTC
TGGGGCGATTATCTGCAATACGATAAGCCGTACTATATGCTGAACCTGTA
TGATCCGAACAAATATGTGGATGTCAATAATGTGGGTATTCGTGGTTACA
TGTATTTGAAGGGTCCGCGTGGCAGCGTTATGACGACCAACATTTACCTG
AACTCTAGCCTGTACCGTGGTACGAAATTCATCATTAAGAAATATGCCAG
CGGCAACAAAGATAACATTGTGCGTAATAACGATCGTGTCTACATCAACG
TGGTCGTGAAGCGTAAAGAGTACCGTCTGGCGACCAACGCTTCGCAGGCG
GGTGTTGAGAAAATTCTGAGCGCGTTGGAGATCCCTCGTGTCCGTCGTCT
GAGCCAAGTCGTGGTTATGAAGAGCAAGAACGACCAGGGTATCACTAACA
AGTGCAAGATGAACCTGCAAGACCGTCGTGGTAACGACATCGGCTTTATT
GGTTTCCACCAGTTCAACAATATTGCTAAACTGGTAGCGAGCAATTGGTA
CAATCGTCAGATTGAGCGCCGTAGCCGTCGTTTGGGCTGTAGCTGGGAGT
TTATCCCGGTCGATGATGGTTGGGGCGAACGTCCGCTG

SEQ ID NO: 10 (Polypeptide Sequence of Modified BoNT/A “Cat-D”)

MPFVNKQFNYKDPVNGVDIAYIKIPNAGQMQPVKAFKIHNKIWVIPERDT
FTNPEEGDLNPPPEAKQVPVSYYDSTYLSTDNEKDNYLKGVTKLFERIYS
TDLGRMLLTSIVRGIPFWGGSTIDTELKVIDTNCINVIQPDGSYRSEELN
LVIIGPSADIIQFECKSFGHEVLNLTRNGYGSTQYIRFSPDFTFGFEESL
EVDTNPLLGAGKFATDPAVTLAHELIHAGHRLYGIAINPNRVFKVNTNAY
YEMSGLEVSFEELRTFGGHDAKFIDSLQENEFRLYYYNKFKDIASTLNKA
KSIVGTTASLQYMKNVFKEKYLLSEDTSGKFSVDKLKFDKLYKMLTEIYT
EDNFVKFFKVLNRKTYLNFDKAVFKINIVPKVNYTIYDGFNLRNTNLAAN
FNGQNTEINNMNFTKLKNFTGLFEFYKLLCVRGIITSKTKSLDKGYNKAL
NDLCIKVNNWDLFFSPSEDNFTNDLNKGEEITSDTNIEAAEENISLDLIQ
QYYLTFNFDNEPENISIENLSSDIIGQLELMPNIERFPNGKKYELDKYTM
FHYLRAQEFEHGKSRIALTNSVNEALLNPSRVYTFFSSDYVKKVNKATEA
AMFLGWVEQLVYDFTDETSEVSTTDKIADITIIIPYIGPALNIGNMLYKD
DFVGALIFSGAVILLEFIPEIAIPVLGTFALVSYIANKVLTVQTIDNALS
KRNEKWDEVYKYIVTNWLAKVNTQIDLIRKKMKEALENQAEATKAIINYQ
YNQYTEEEKNNINFNIDDLSSKLNESINKAMININKFLNQCSVSYLMNSM
IPYGVKRLEDFDASLKDALLKYIYDNRGTLIGQVDRLKDKVNNTLSTDIP
FQLSKYVDNQRLLSTFTEYIKNIINTSILNLRYESNHLIDLSRYASKINI
GSKVNFDPIDKNQIQLFNLESSKIEVILKNAIVYNSMYENFSTSFWIRIP
KYFNSISLNNEYTIINCMENNSGWKVSLNYGEIIWTLQDTQEIKQRVVFK
YSQMINISDYINRWIFVTITNNRLNNSKIYINGRLIDQKPISNLGNIHAS
NNIMFKLDGCRDTHRYIWIKYFNLFDKELNEKEIKDLYDNQSNSGILKDF
WGDYLQYDKPYYMLNLYDPNKYVDVNNVGIRGYMYLKGPRGSVMTTNIYL
NSSLYRGTKFIIKKYASGNKDNIVRNNDRVYINVVVKRKEYRLATNASQA
GVEKILSALEIPRVRRLSQVVVMKSKNDQGITNKCKMNLQDRRGNDIGFI
GFHQFNNIAKLVASNWYNRQIERRSRRLGCSWEFIPVDDGWGERPL

SEQ ID NO: 11 (Polypeptide Sequence of Modified BoNT/A “Chimera 1”)

MPFVNKQFNYKDPVNGVDIAYIKIPNAGQMQPVKAFKIHNKIWVIPERDT
FTNPEEGDLNPPPEAKQVPVSYYDSTYLSTDNEKDNYLKGVTKLFERIYS
TDLGRMLLTSIVRGIPFWGGSTIDTELKVIDTNCINVIQPDGSYRSEELN
LVIIGPSADIIQFECKSFGHEVLNLTRNGYGSTQYIRFSPDFTFGFEESL
EVDTNPLLGAGKFATDPAVTLAHELIHAGHRLYGIAINPNRVFKVNTNAY
YEMSGLEVSFEELRTFGGHDAKFIDSLQENEFRLYYYNKFKDIASTLNKA
KSIVGTTASLQYMKNVFKEKYLLSEDTSGKFSVDKLKFDKLYKMLTEIYT
EDNFVKFFKVLNRKTYLNFDKAVFKINIVPKVNYTIYDGFNLRNTNLAAN
FNGQNTEINNMNFTKLKNFTGLFEFYKLLCVRGIITSKTKSLDKGYNKAL
NDLCIKVNNWDLFFSPSEDNFTNDLNKGEEITSDTNIEAAEENISLDLIQ
QYYLTFNFDNEPENISIENLSSDIIGQLELMPNIERFPNGKKYELDKYTM
FHYLRAQEFEHGKSRIALTNSVNEALLNPSRVYTFFSSDYVKKVNKATEA
AMFLGWVEQLVYDFTDETSEVSTTDKIADITIIIPYIGPALNIGNMLYKD
DFVGALIFSGAVILLEFIPEIAIPVLGTFALVSYIANKVLTVQTIDNALS
KRNEKWDEVYKYIVTNWLAKVNTQIDLIRKKMKEALENQAEATKAIINYQ
YNQYTEEEKNNINFNIDDLSSKLNESINKAMININKFLNQCSVSYLMNSM
IPYGVKRLEDFDASLKDALLKYIYDNRGTLIGQVDRLKDKVNNTLSTDIP
FQLSKYVDNQRLLSTFTEYIKSEILNNIILNLRYKDNNLIDLSGYGAKVE
VYDGVELNDKNQFKLTSSANSKIRVTQNQNIIFNSVFLDFSVSFWIRIPK
YKNDGIQNYIHNEYTIINCMKNNSGWKISIRGNRIIWTLIDINGKTKSVF
FEYNIREDISEYINRWFFVTITNNLNNAKIYINGKLESNTDIKDIREVIA
NGEIIFKLDGDIDRTQFIWMKYFSIFNTELSQSNIEERYKIQSYSEYLKD
FWGNPLMYNKEYYMFNAGNKNSYIKLKKDSPVGEILTRSKYNQNSKYINY
RDLYIGEKFIIRRKSNSQSINDDIVRKEDYIYLDFFNLNQEWRVYTYKYF
KKEEMKLFLAPIYDSDEFYNTIQIKEYDEQPTYSCQLLFKKDEESTDEIG
LIGIHRFYESGIVFEEYKDYFCISKWYLKEVKRKPYNLKLGCNWQFIPKD
EGWTEHHHHHHHHHH

SEQ ID NO: 12 (Polypeptide Sequence of Modified BoNT/A “Chimera 2”)

MPFVNKQFNYKDPVNGVDIAYIKIPNAGQMQPVKAFKIHNKIWVIPERDT
FTNPEEGDLNPPPEAKQVPVSYYDSTYLSTDNEKDNYLKGVTKLFERIYS
TDLGRMLLTSIVRGIPFWGGSTIDTELKVIDTNCINVIQPDGSYRSEELN
LVIIGPSADIIQFECKSFGHEVLNLTRNGYGSTQYIRFSPDFTFGFEESL
EVDTNPLLGAGKFATDPAVTLAHELIHAGHRLYGIAINPNRVFKVNTNAY
YEMSGLEVSFEELRTFGGHDAKFIDSLQENEFRLYYYNKFKDIASTLNKA
KSIVGTTASLQYMKNVFKEKYLLSEDTSGKFSVDKLKFDKLYKMLTEIYT
EDNFVKFFKVLNRKTYLNFDKAVFKINIVPKVNYTIYDGFNLRNTNLAAN
FNGQNTEINNMNFTKLKNFTGLFEFYKLLCVRGIITSKTKSLDKGYNKAL
NDLCIKVNNWDLFFSPSEDNFTNDLNKGEEITSDTNIEAAEENISLDLIQ
QYYLTFNFDNEPENISIENLSSDIIGQLELMPNIERFPNGKKYELDKYTM
FHYLRAQEFEHGKSRIALTNSVNEALLNPSRVYTFFSSDYVKKVNKATEA
AMFLGWVEQLVYDFTDETSEVSTTDKIADITIIIPYIGPALNIGNMLYKD
DFVGALIFSGAVILLEFIPEIAIPVLGTFALVSYIANKVLTVQTIDNALS
KRNEKWDEVYKYIVTNWLAKVNTQIDLIRKKMKEALENQAEATKAIINYQ
YNQYTEEEKNNINFNIDDLSSKLNESINKAMININKFLNQCSVSYLMNSM
IPYGVKRLEDFDASLKDALLKYIYDNRGTLIGQVDRLKDKVNNTLSTDIP
FQLSKYVDNQRLLSTFTEYIKNIIELGGGGSELSEILNNIILNLRYKDNN
LIDLSGYGAKVEVYDGVELNDKNQFKLTSSANSKIRVTQNQNIIFNSVFL
DFSVSFWIRIPKYKNDGIQNYIHNEYTIINCMKNNSGWKISIRGNRIIWT
LIDINGKTKSVFFEYNIREDISEYINRWFFVTITNNLNNAKIYINGKLES
NTDIKDIREVIANGEIIFKLDGDIDRTQFIWMKYFSIFNTELSQSNIEER
YKIQSYSEYLKDFWGNPLMYNKEYYMFNAGNKNSYIKLKKDSPVGEILTR
SKYNQNSKYINYRDLYIGEKFIIRRKSNSQSINDDIVRKEDYIYLDFFNL
NQEWRVYTYKYFKKEEMKLFLAPIYDSDEFYNTIQIKEYDEQPTYSCQLL
FKKDEESTDEIGLIGIHRFYESGIVFEEYKDYFCISKWYLKEVKRKPYNL
KLGCNWQFIPKDEGWTEHHHHHHHHHH

SEQ ID NO: 13 (Polypeptide Sequence of Modified BoNT/A “Chimera 3A”)

MPFVNKQFNYKDPVNGVDIAYIKIPNAGQMQPVKAFKIHNKIWVIPERDT
FTNPEEGDLNPPPEAKQVPVSYYDSTYLSTDNEKDNYLKGVTKLFERIYS
TDLGRMLLTSIVRGIPFWGGSTIDTELKVIDTNCINVIQPDGSYRSEELN
LVIIGPSADIIQFECKSFGHEVLNLTRNGYGSTQYIRFSPDFTFGFEESL
EVDTNPLLGAGKFATDPAVTLAHELIHAGHRLYGIAINPNRVFKVNTNAY
YEMSGLEVSFEELRTFGGHDAKFIDSLQENEFRLYYYNKFKDIASTLNKA
KSIVGTTASLQYMKNVFKEKYLLSEDTSGKFSVDKLKFDKLYKMLTEIYT
EDNFVKFFKVLNRKTYLNFDKAVFKINIVPKVNYTIYDGFNLRNTNLAAN
FNGQNTEINNMNFTKLKNFTGLFEFYKLLCVRGIITSKTKSLDKGYNKAL
NDLCIKVNNWDLFFSPSEDNFTNDLNKGEEITSDTNIEAAEENISLDLIQ
QYYLTFNFDNEPENISIENLSSDIIGQLELMPNIERFPNGKKYELDKYTM
FHYLRAQEFEHGKSRIALTNSVNEALLNPSRVYTFFSSDYVKKVNKATEA
AMFLGWVEQLVYDFTDETSEVSTTDKIADITIIIPYIGPALNIGNMLYKD
DFVGALIFSGAVILLEFIPEIAIPVLGTFALVSYIANKVLTVQTIDNALS
KRNEKWDEVYKYIVTNWLAKVNTQIDLIRKKMKEALENQAEATKAIINYQ
YNQYTEEEKNNINFNIDDLSSKLNESINKAMININKFLNQCSVSYLMNSM
IPYGVKRLEDFDASLKDALLKYIYDNRGTLIGQVDRLKDKVNNTLSTDIP
FQLSKYVDNQRLLSTFTEYIKNILNNIILNLRYKDNNLIDLSGYGAKVEV
YDGVELNDKNQFKLTSSANSKIRVTQNQNIIFNSVFLDFSVSFWIRIPKY
KNDGIQNYIHNEYTIINCMKNNSGWKISIRGNRIIWTLIDINGKTKSVFF
EYNIREDISEYINRWFFVTITNNLNNAKIYINGKLESNTDIKDIREVIAN
GEIIFKLDGDIDRTQFIWMKYFSIFNTELSQSNIEERYKIQSYSEYLKDF
WGNPLMYNKEYYMFNAGNKNSYIKLKKDSPVGEILTRSKYNQNSKYINYR
DLYIGEKFIIRRKSNSQSINDDIVRKEDYIYLDFFNLNQEWRVYTYKYFK
KEEMKLFLAPIYDSDEFYNTIQIKEYDEQPTYSCQLLFKKDEESTDEIGL
IGIHRFYESGIVFEEYKDYFCISKWYLKEVKRKPYNLKLGCNWQFIPKDE
GWTEHHHHHHHHHH

SEQ ID NO: 14 (Polypeptide Sequence of Modified BoNT/A “Chimera 3B”)

MPFVNKQFNYKDPVNGVDIAYIKIPNAGQMQPVKAFKIHNKIWVIPERDT
FTNPEEGDLNPPPEAKQVPVSYYDSTYLSTDNEKDNYLKGVTKLFERIYS
TDLGRMLLTSIVRGIPFWGGSTIDTELKVIDTNCINVIQPDGSYRSEELN
LVIIGPSADIIQFECKSFGHEVLNLTRNGYGSTQYIRFSPDFTFGFEESL
EVDTNPLLGAGKFATDPAVTLAHELIHAGHRLYGIAINPNRVFKVNTNAY
YEMSGLEVSFEELRTFGGHDAKFIDSLQENEFRLYYYNKFKDIASTLNKA
KSIVGTTASLQYMKNVFKEKYLLSEDTSGKFSVDKLKFDKLYKMLTEIYT
EDNFVKFFKVLNRKTYLNFDKAVFKINIVPKVNYTIYDGFNLRNTNLAAN
FNGQNTEINNMNFTKLKNFTGLFEFYKLLCVRGIITSKTKSLDKGYNKAL
NDLCIKVNNWDLFFSPSEDNFTNDLNKGEEITSDTNIEAAEENISLDLIQ
QYYLTFNFDNEPENISIENLSSDIIGQLELMPNIERFPNGKKYELDKYTM
FHYLRAQEFEHGKSRIALTNSVNEALLNPSRVYTFFSSDYVKKVNKATEA
AMFLGWVEQLVYDFTDETSEVSTTDKIADITIIIPYIGPALNIGNMLYKD
DFVGALIFSGAVILLEFIPEIAIPVLGTFALVSYIANKVLTVQTIDNALS
KRNEKWDEVYKYIVTNWLAKVNTQIDLIRKKMKEALENQAEATKAIINYQ
YNQYTEEEKNNINFNIDDLSSKLNESINKAMININKFLNQCSVSYLMNSM
IPYGVKRLEDFDASLKDALLKYIYDNRGTLIGQVDRLKDKVNNTLSTDIP
FQLSKYVDNQRLLSTFTEYIKNILNNIILNLRYKDNNLIDLSGYGAKVEV
YDGVELNDKNQFKLTSSANSKIRVTQNQNIIFNSVFLDFSVSFWIRIPKY
KNDGIQNYIHNEYTIINCMKNNSGWKISIRGNRIIWTLIDINGKTKSVFF
EYNIREDISEYINRWFFVTITNNLNNAKIYINGKLESNTDIKDIREVIAN
GEIIFKLDGDIDRTQFIWMKYFSIFNTELSQSNIEERYKIQSYSEYLKDF
WGNPLMYNKEYYMFNAGNKNSYIKLKKDSPVGEILTRSKYNQNSKYINYR
DLYIGEKFIIRRKSNSQSINDDIVRKEDYIYLDFFNLNQEWRVYTYKYFK
KEEMKLFLAPIYDSDEFYNTIQIKEYDEQPTYSCQLLFKKDEESTDEIGL
IGIHRFYESGIVFEEYKDYFCISKWYLKEVKRKPYNLKLGCNWQFIPKDE
GWTE

SEQ ID NO: 15 (Polypeptide Sequence of Modified BoNT/A “Chimera 3C”)

MPFVNKQFNYKDPVNGVDIAYIKIPNAGQMQPVKAFKIHNKIWVIPERDT
FTNPEEGDLNPPPEAKQVPVSYYDSTYLSTDNEKDNYLKGVTKLFERIYS
TDLGRMLLTSIVRGIPFWGGSTIDTELKVIDTNCINVIQPDGSYRSEELN
LVIIGPSADIIQFECKSFGHEVLNLTRNGYGSTQYIRFSPDFTFGFEESL
EVDTNPLLGAGKFATDPAVTLAHELIHAGHRLYGIAINPNRVFKVNTNAY
YEMSGLEVSFEELRTFGGHDAKFIDSLQENEFRLYYYNKFKDIASTLNKA
KSIVGTTASLQYMKNVFKEKYLLSEDTSGKFSVDKLKFDKLYKMLTEIYT
EDNFVKFFKVLNRKTYLNFDKAVFKINIVPKVNYTIYDGFNLRNTNLAAN
FNGQNTEINNMNFTKLKNFTGLFEFYKLLCVRGIITSKTKSLDKGYNKAL
NDLCIKVNNWDLFFSPSEDNFTNDLNKGEEITSDTNIEAAEENISLDLIQ
QYYLTFNFDNEPENISIENLSSDIIGQLELMPNIERFPNGKKYELDKYTM
FHYLRAQEFEHGKSRIALTNSVNEALLNPSRVYTFFSSDYVKKVNKATEA
AMFLGWVEQLVYDFTDETSEVSTTDKIADITIIIPYIGPALNIGNMLYKD
DFVGALIFSGAVILLEFIPEIAIPVLGTFALVSYIANKVLTVQTIDNALS
KRNEKWDEVYKYIVTNWLAKVNTQIDLIRKKMKEALENQAEATKAIINYQ
YNQYTEEEKNNINFNIDDLSSKLNESINKAMININKFLNQCSVSYLMNSM
IPYGVKRLEDFDASLKDALLKYIYDNRGTLIGQVDRLKDKVNNTLSTDIP
FQLSKYVDNQRLLSTFTEYIKNILNNIILNLRYKDNNLIDLSGYGAKVEV
YDGVELNDKNQFKLTSSANSKIRVTQNQNIIFNSVFLDFSVSFWIRIPKY
KNDGIQNYIHNEYTIINCMKNNSGWKISIRGNRIIWTLIDINGKTKSVFF
EYNIREDISEYINRWFFVTITNNLNNAKIYINGKLESNTDIKDIREVIAN
GEIIFKLDGDIDRTQFIWMKYFSIFNTELSQSNIEERYKIQSYSEYLKDF
WGNPLMYNKEYYMFNAGNKNSYIKLKKDSPVGEILTRSKYNQNSKYINYR
DLYIGEKFIIRRKSNSQSINDDIVRKEDYIYLDFFNLNQEWRVYTYKYFK
KEEEKLFLAPISDSDEFYNTIQIKEYDEQPTYSCQLLFKKDEESTDEIGL
IGIHRFYESGIVFEEYKDYFCISKWYLKEVKRKPYNLKLGCNWQFIPKDE
GWTE

SEQ ID NO: 16 (Polypeptide Sequence of BoNT/B)

MPVTINNFNYNDPIDNNNIIMMEPPFARGTGRYYKAFKITDRIWIIPERY
TFGYKPEDFNKSSGIFNRDVCEYYDPDYLNTNDKKNIFLQTMIKLFNRIK
SKPLGEKLLEMIINGIPYLGDRRVPLEEFNTNIASVTVNKLISNPGEVER
KKGIFANLIIFGPGPVLNENETIDIGIQNHFASREGFGGIMQMKFCPEYV
SVFNNVQENKGASIFNRRGYFSDPALILMHELIHVLHGLYGIKVDDLPIV
PNEKKFFMQSTDAIQAEELYTFGGQDPSIITPSTDKSIYDKVLQNFRGIV
DRLNKVLVCISDPNININIYKNKFKDKYKFVEDSEGKYSIDVESFDKLYK
SLMFGFTETNIAENYKIKTRASYFSDSLPPVKIKNLLDNEIYTIEEGFNI
SDKDMEKEYRGQNKAINKQAYEEISKEHLAVYKIQMCKSVKAPGICIDVD
NEDLFFIADKNSFSDDLSKNERIEYNTQSNYIENDFPINELILDTDLISK
IELPSENTESLTDFNVDVPVYEKQPAIKKIFTDENTIFQYLYSQTFPLDI
RDISLTSSFDDALLFSNKVYSFFSMDYIKTANKVVEAGLFAGWVKQIVND
FVIEANKSNTMDKIADISLIVPYIGLALNVGNETAKGNFENAFEIAGASI
LLEFIPELLIPVVGAFLLESYIDNKNKIIKTIDNALTKRNEKWSDMYGLI
VAQWLSTVNTQFYTIKEGMYKALNYQAQALEEIIKYRYNIYSEKEKSNIN
IDFNDINSKLNEGINQAIDNINNFINGCSVSYLMKKMIPLAVEKLLDFDN
TLKKNLLNYIDENKLYLIGSAEYEKSKVNKYLKTIMPFDLSIYTNDTILI
EMFNKYNSEILNNIILNLRYKDNNLIDLSGYGAKVEVYDGVELNDKNQFK
LTSSANSKIRVTQNQNIIFNSVFLDFSVSFWIRIPKYKNDGIQNYIHNEY
TIINCMKNNSGWKISIRGNRIIWTLIDINGKTKSVFFEYNIREDISEYIN
RWFFVTITNNLNNAKIYINGKLESNTDIKDIREVIANGEIIFKLDGDIDR
TQFIWMKYFSIFNTELSQSNIEERYKIQSYSEYLKDFWGNPLMYNKEYYM
FNAGNKNSYIKLKKDSPVGEILTRSKYNQNSKYINYRDLYIGEKFIIRRK
SNSQSINDDIVRKEDYIYLDFFNLNQEWRVYTYKYFKKEEEKLFLAPISD
SDEFYNTIQIKEYDEQPTYSCQLLFKKDEESTDEIGLIGIHRFYESGIVF
EEYKDYFCISKWYLKEVKRKPYNLKLGCNWQFIPKDEGWTE

### EXAMPLES

### Example 1

### Cloning, Expression and Purification

The nucleotide sequence SEQ ID NO: 1, which encodes wild-type BoNT/A (SEQ ID NO: 2) was mutated to introduce the following substitutions to form the four constructs shown in Table 1 below:

TABLE 1

Constructs.

| Construct | Mutations | Nucleotide Sequence | Polypeptide Sequence |
|---|---|---|---|
| Cat-A | N930K, S955K, Q991K, N1026K, N1052K, Q1229K, N886K | 3 | 4 |
| Cat-B | N930K, S955K, Q991K, N1026K, N1052K, Q1229K, N954K | 5 | 6 |
| Cat-C | N930K, S955K, Q991K, N1026K, N1052K, Q1229K, N1025K | 7 | 8 |
| Cat-D* | N1188R, D1213R, G1215R, N1216R, N1242R, N1243R, S1274R, T1277R | 9 | 10 |

*Cat-D had a calculated pl of 7.45, and a molecular weight of 149,859.

DNA constructs encoding the modified BoNT/A molecules above were synthesised, cloned into the pJ401 expression vector and then transformed into BL21 (DE3) E. coli. This allowed for soluble over-expression of the recombinant Cat-A, Cat-B, Cat-C, and Cat-D proteins in BL21(DE3) E. coli.

The recombinant modified BoNTs were purified using classical chromatography techniques from the E. coli lysates. An initial purification step using a cation-exchange resin was employed, followed by an intermediate purification step using a hydrophobic interaction resin.

The recombinant modified BoNT single-chain was then cleaved by proteolysis, resulting in the activated di-chain modified BoNT. A final purification step was then employed to remove remaining contaminants. Suitable techniques are taught in WO2015/166242, W02017055274A1, EP2524963B1, EP2677029B1, and US10087432B2.

### Example 2

### Characterization of Purified Modified BoNT/A

The modified BoNTs described in Example 1 above were characterised experimentally as follows.

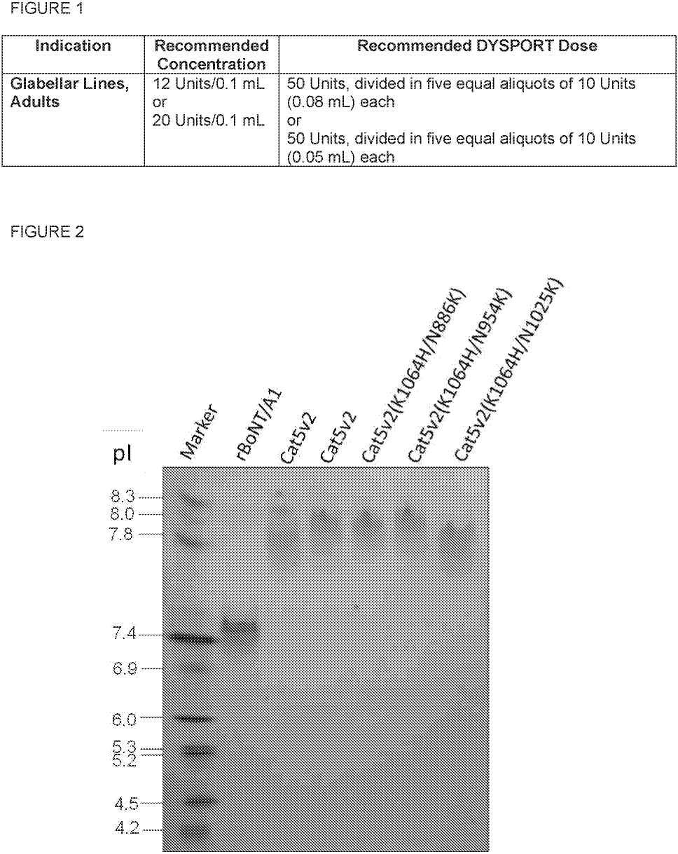

Measurement of the pl showed that the modified BoNTs had an isoelectric point greater than that of unmodified (native) BoNT/A1 - see FIG. 2 and Table 2 below.

TABLE 2

Modified BoNT/A pl values.

| BoNT/A1 molecule | pl (calculated) | pl (observed) |
|---|---|---|
| Modified, “Cat-A” [Cat5v2(K1064H/N886K] (SEQ ID NO: 4) | 6.9 | ~8.0 |
| Modified, “Cat-B” [Cat5v2(K1064/N954K)] (SEQ ID NO: 6) | 6.9 | ~8.0 |
| Modified, “Cat-C” [Cat5v2(K1064H/N1025K)] (SEQ ID NO: 8) | 6.9 | 7.8-8.0 |
| Native BoNT/A1 [rBoNT/A1] (SEQ ID NO: 2) | 6.05 | ~7.4 |

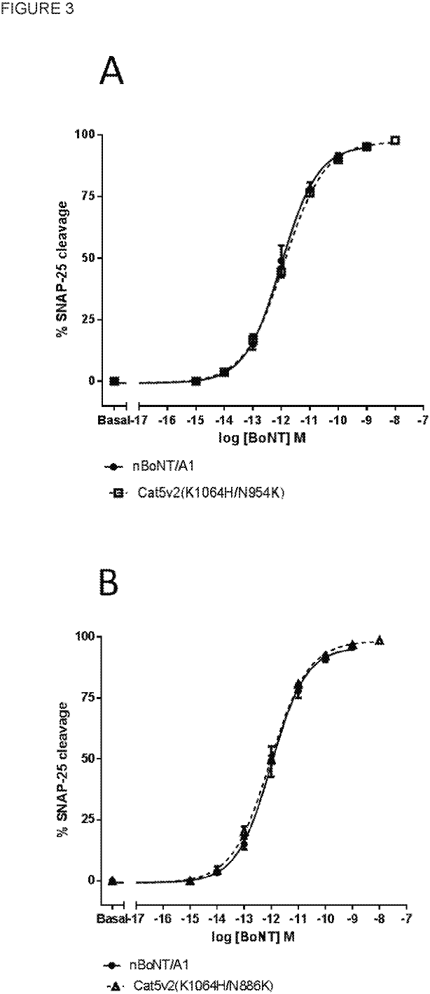

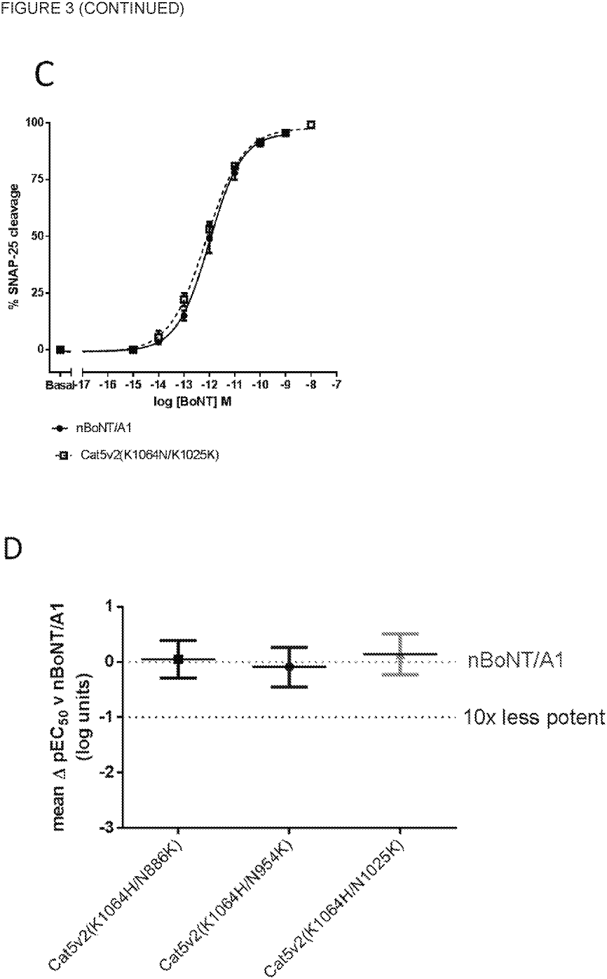

The ability of the modified BoNTs to enter neurons and cleave SNAP-25 (the target of BoNT/A1) was assessed using rat embryonic spinal cord neurons (eSCN). FIG. 3 shows that the modified BoNTs retained the same ability to enter the neuron and cleave SNAP-25 as native BoNT/A1.

Potency of the modified BoNTs was further assessed using the mouse phrenic nerve hemi-diaphragm assay (mPNHD). FIG. 4 shows that the modified BoNTs retained the same ability to inhibit the contractile abilities of the mouse hemi-diaphragm as native BoNT/A1.

The in vivo mouse Digital Abduction Score (DAS) assay was used to assess potency as well as safety relative to native BoNT/A1. Both molecules (Cat-A [SEQ ID NO: 4] and Cat-B [SEQ ID NO: 6]) displayed a higher safety ratio relative to native BoNT/A1 and were slightly more potent. These data are presented in Table 3 below:

TABLE 3

DAS assay and safety ratio.

| Molecule | DAS ED₅₀ (pg/mouse) | Dose DAS 4 (pg/mouse) | Dose for -10% ΔBW (pg/mouse) | Safety Ratio |
|---|---|---|---|---|
| Native BoNT/A1 (n=5) (SEQ ID NO: 2) | 2 | 10-20 | 9.9-14.5 | 7 |
| Modified, “Cat-A” (SEQ ID NO: 4) | 1.16 | 10-20 | 27.4 | 24 |
| Modified, “Cat-B” (SEQ ID NO: 6) | 1.79 | 25 | 47.6 | 27 |

-DAS ED₅₀: Calculated dose inducing a DAS 2
-Dose DAS 4: Experimental dose inducing a DAS 4
-BW: Body weight
-Dose for -10% ΔBW: Calculated dose inducing a decrease of 10% on BW in comparison to BW at D0
-Safety Ratio: Dose for -10% ΔBW / DAS ED₅₀

The Safety Ratio is a measure of a negative effect of BoNT treatment (weight loss) with respect to potency (half maximal digital abduction score (DAS)). It is calculated as the ratio between -10% Body Weight (BW) and the DAS ED₅₀, where -10%BW refers to the amount of BoNT (pg/animal) required for a 10% decrease in body weight, and ED₅₀ refers to the amount of BoNT (pg/animal) that will produce a DAS of 2.

The DAS assay is performed by injection of 20µl of modified BoNT/A, formulated in Gelatin Phosphate Buffer, into the mouse gastrocnemius/soleus complex, followed by assessment of Digit Abduction as previously reported by Aoki (Aoki KR, Toxicon 39: 1815-1820; 2001).

### Example 3

### Cloning, Expression and Purification of BoNT/AB Chimeras

BoNT/AB chimeric constructs 1, 2, 3A, 3B, and 3C (SEQ ID NO: 11 to 15, respectively) were constructed from DNA encoding the parent serotype molecule and appropriate oligonucleotides using standard molecular biology techniques. These were then cloned into the pJ401 expression vector with or without a C-terminal His₁₀-tag and transformed into BLR (DE3) E. coli cells for over-expression. These cells were grown at 37° C. and 225 RPM shaking in 2 L baffled conical flasks containing 1 L modified Terrific Broth (mTB) supplemented with the appropriate antibiotic. Once the A₆₀₀ reached >0.5, the incubator temperature was decreased to 16° C., and then induced with 1 mM IPTG an hour later for 20 h at 225 RPM shaking, to express the recombinant BoNT/AB construct.

Harvested cells were lysed by ultrasonication and clarified by centrifugation at 4500 RPM for 1 h at 4° C. The recombinant BoNT/AB chimeric molecules were then extracted in ammonium sulphate and purified by standard fast protein liquid chromatography (FPLC) techniques. This involved using a hydrophobic interaction resin for capture and an anion-exchange resin for the intermediate purification step. The partially purified molecules were then proteolytically cleaved with endoproteinase Lys-C to yield the active di-chain. This was further purified with a second hydrophobic interaction resin to obtain the final BoNT/AB chimera.

For BoNT/AB chimeric molecules with a decahistadine tag (H₁₀) (chimera 1, 2, 3A), the capture step employed the use of an immobilised nickel resin instead of the hydrophobic interaction resin.

The sequence of each chimera is presented in Table 4.

TABLE 4

chimeric BoNT/AB constructs

| Molecule | SEQ ID NO | Sequence |
|---|---|---|
| Chimera 1 | 11 | A1:1-871 + B1:858-1291 (E1191M/S1199Y) + His₁₀-tag |
| Chimera 2 | 12 | A1:1-874 + ELGGGGSEL + B1:858-1291 (E1191M/S1199Y) + His₁₀-tag |
| Chimera 3A | 13 | A1:1-872 + B1: 860-1291 (E1191M/S1199Y) + His₁₀-tag |
| Chimera 3B | 14 | A1:1-872 + B1: 860-1291 (E1191M/S1199Y) |
| Chimera 3C | 15 | A1:1-872 + B1: 860-1291 |

### Example 4

### Comparison of BoNT/AB Chimera 1, 2 and 3A

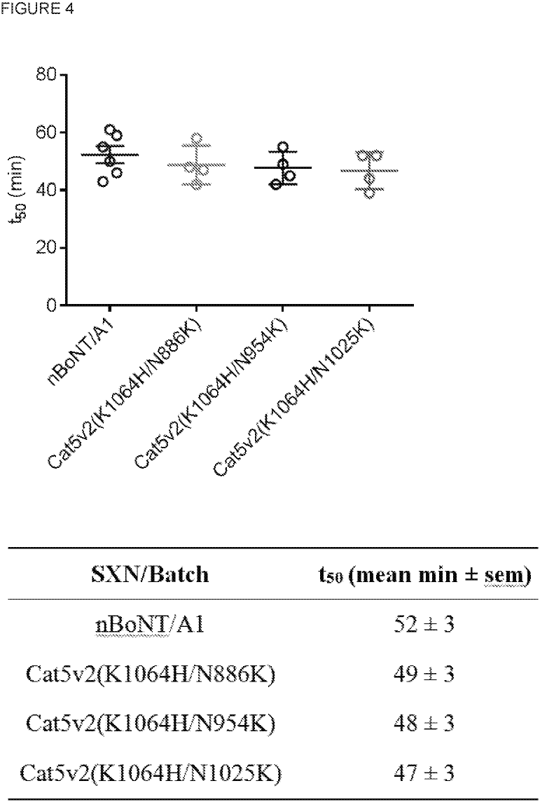

BoNT/AB chimera 1, 2 and 3A which have a C-terminal His₁₀ tag and E1191M/S1199Y double mutation were purified as described in Example 3 (FIG. 5) and tested for functional activity.

### Rat Spinal Cord Neurons Snap-25 Cleavage Assay

Primary cultures of rat spinal cord neurons (SCN) were prepared and grown, for 3 weeks, in 96 well tissue culture plates (as described in: Masuyer et al., 2011, J. Struct. Biol. Structure and activity of a functional derivative of Clostridium botulinum neurotoxin B; and in: Chaddock et al., 2002, Protein Expr. Purif. Expression and purification of catalytically active, non-toxic endopeptidase derivatives of Clostridium botulinum toxin type A). Serial dilutions of BoNT/AB were prepared in SCN feeding medium. The growth medium from the wells to be treated was collected and filtered (0.2 µm filter). 125 µL of the filtered medium was added back to each test well. 125 µL of diluted toxin was then added to the plate (triplicate wells).

The treated cells were incubated at 37° C., 10% CO₂, for 24 ± 1 h).

### Analysis of BoNT Activity Using the SNAP-25 Cleavage Assay

Following treatment, BoNT was removed and cells were washed once in PBS (Gibco, UK). Cells were lysed in 1x NuPAGE lysis buffer (Life Technologies) supplemented with 0.1 M dithiothreitol (DTT) and 250 units/mL benzonase (Sigma). Lysate proteins were separated by SDS-PAGE and transferred to nitrocellulose membranes. Membranes were probed with a primary antibody specific for SNAP-25 (Sigma #S9684) which recognizes uncleaved SNAP-25 as well as SNAP-25 cleaved by the BoNT/A endopeptidase. The secondary antibody used was an HRP-conjugated anti-rabbit IgG (Sigma #A6154). Bands were detected by enhanced chemiluminescence and imaged using a pXi6 Access (Synoptics, UK). The intensity of bands was determined using GeneTools software (Syngene, Cambridge, UK) and the percentage of SNAP-25 cleaved at each concentration of BoNT calculated. Data were fitted to a 4-parameter logistic equation and pEC₅₀ calculated using GraphPad Prism version 6 (GraphPad).

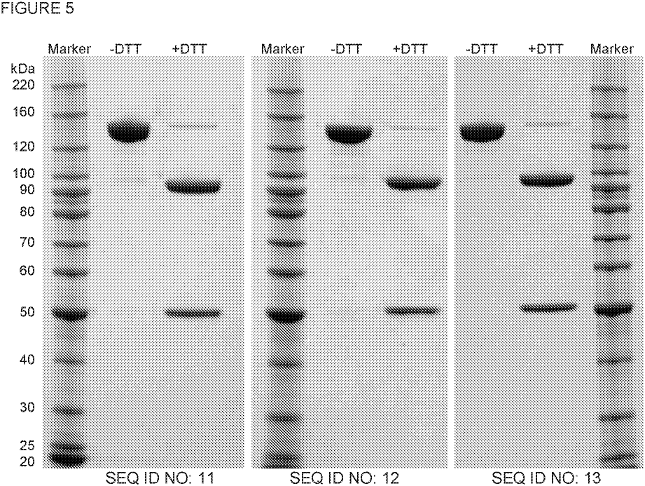

Table 5 below provides the pEC₅₀ values determined for Chimera 1, 2 and 3A in the rat SCN SNAP-25 cleavage assay. These results show that the three BoNT/AB chimeras retained the ability to enter rat spinal cord neurons and cleave their target substrate. However, chimera 3A was more potent than chimera 1 and 2 in this assay (see also FIG. 6).

TABLE 5

pEC₅₀ values.

| | pEC₅₀ ±SEM |
|---|---|
| Chimera 1 | 12.42 ±0.04 |
| Chimera 2 | 12.57 ±0.01 |
| Chimera 3A | 12.89 ±0.04 |

### Digit Abduction Scoring (DAS) Assay

The method to measure the activity of BoNT/AB chimera 1, 2 and 3A in the DAS assay is based on the startled response toe spreading reflex of mice, when suspended briefly by the tail. This reflex is scored as Digit Abduction Score (DAS) and is inhibited after administration of BoNT into the gastrocnemius-soleus muscles of the hind paw. Mice are suspended briefly by the tail to elicit a characteristic startled response in which the animal extends its hind limb and abducts its hind digits. (Aoki et al. 1999, Eur. J. Neurol.; 6 (suppl. 4) S3-S10).

On the day of injection, mice were anaesthetized in an induction chamber receiving isoflurane 3% in oxygen. Each mouse received an intramuscular injection of BoNT/AB chimera or vehicle (phosphate buffer containing 0.2 % gelatine) in the gastrocnemius-soleus muscles of the right hind paw.

Following neurotoxin injection, the varying degrees of digit abduction were scored on a scale from zero to four, where 0= normal and 4= maximal reduction in digit abduction and leg extension. ED50 was determined by nonlinear adjustment analysis using average of maximal effect at each dose. The mathematical model used was the 4 parameters logistic model.

DAS was performed every 2 hours during the first day after dosing; thereafter it was performed 3 times a day for 4 days.

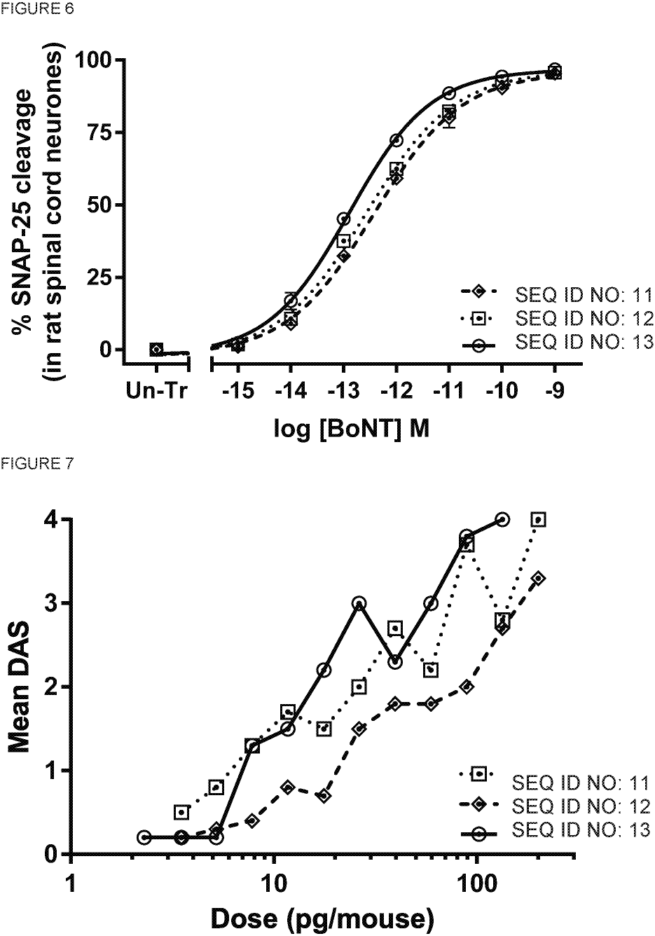

FIG. 7 shows the fitted curves for chimera 1, 2 and 3A (SEQ ID NO: 11, 12 and 13 respectively). The chimera 3A curve is shifted to the left, meaning lower doses of chimera 3A achieved a similar DAS response compared to chimera 1 and 2, therefore showing that chimera 3A is more potent than the others in the mouse DAS assay; see also the table below (Table 6) that provides the values for the calculated ED50 and the dose leading to DAS 4 (highest score) for each chimera.

Table 6 below provides the ED₅₀ and DAS 4 doses determined for unmodified recombinant BoNT/A1 (rBoNT/A1 — SEQ ID NO: 2) and chimeras 1, 2 and 3A in the mouse DAS assay. These results show that of the three chimeras, chimera 3A has the highest in vivo potency in inducing muscle weakening. Studies shown in FIG. 7 and Table 6 were performed in mice obtained from Charles River laboratories.

TABLE 6

ED₅₀ values.

| | ED₅₀ (pg/mouse) | DAS 4 dose (pg/mouse) |
|---|---|---|
| rBoNT/A1 | 1 | 5 |
| Chimera 1 | 23 | 200 |
| Chimera 2 | 89 | >300 |
| Chimera 3A | 18 | 133 |

### Example 5

### Comparison of BoNT/AB Chimera 3B, 3C and Unmodified BoNT/A1

Untagged BoNT/AB chimera 3B and 3C, respectively with and without the presence of the E1191M/S1199Y double mutation (SEQ ID NO: 14 and 15) were purified as described in Example 3 (FIG. 8), and tested for functional activity using unmodified BoNT/A (SEQ ID NO: 2) as a reference.

### Human Pluripotent Stem Cells Snap-25 Cleavage Assay

Cryopreserved PERI.4U-cells were purchased from Axiogenesis (Cologne, Germany). Thawing and plating of the cells were performed as recommended by the manufacturer. Briefly, cryovials containing the cells were thawed in a water bath at 37° C. for 2 minutes. After gentle resuspension the cells were transferred to a 50 mL tube. The cryovial was washed with 1 mL of Peri.4U® thawing medium supplied by the manufacturer and the medium was transfered drop-wise to the cell suspension to the 50 mL tube, prior to adding a further 2 mL of Peri.4U® thawing medium drop-wise to the 50 mL tube. Cells were then counted using a hemocytometer. After this, a further 6 mL of Peri.4U® thawing medium was added to the cell suspension. A cell pellet was obtained by centrifugation at 260 xg (e.g. 1,100 RPM) for 6 minutes at room temperature. Cells were then resuspended in complete Peri.4U® culture medium supplied by the manufacturer. Cells were plated at a density of 50,000 to 150,000 cells per cm² on cell culture plates coated with poly-L-ornithine and laminin. Cells were cultured at 37° C. in a humidified CO₂ atmosphere, and medium was changed completely every 2-3 days during culture.

For toxin treatment, serial dilutions of BoNTs were prepared in Peri.4U® culture medium. The medium from the wells to be treated was collected and filtered (0.2 µm filter). 125 µL of the filtered medium was added back to each test well. 125 µL of diluted toxin was then added to the plate (triplicate wells). The treated cells were incubated at 37° C., 10% CO₂, for 48 ± 1 h).

### Analysis of BoNT Activity Using the SNAP-25 Cleavage Assay

Following treatment, BoNT was removed and cells were washed once in PBS (Gibco, UK). Cells were lysed in 1x NuPAGE lysis buffer (Life Technologies) supplemented with 0.1 M dithiothreitol (DTT) and 250 units/mL benzonase (Sigma). Lysate proteins were separated by SDS-PAGE and transferred to nitrocellulose membranes. Membranes were probed with a primary antibody specific for SNAP-25 (Sigma #S9684) which recognizes uncleaved SNAP-25 as well as SNAP-25 cleaved by the BoNT/A endopeptidase. The secondary antibody used was an HRP-conjugated anti-rabbit IgG (Sigma #A6154). Bands were detected by enhanced chemiluminescence and imaged using a pXi6 Access (Synoptics, UK). The intensity of bands was determined using GeneTools software (Syngene, Cambridge, UK) and the percentage of SNAP-25 cleaved at each concentration of BoNT calculated. Data were fitted to a 4-parameter logistic equation and pEC₅₀ calculated using GraphPad Prism version 6 (GraphPad).

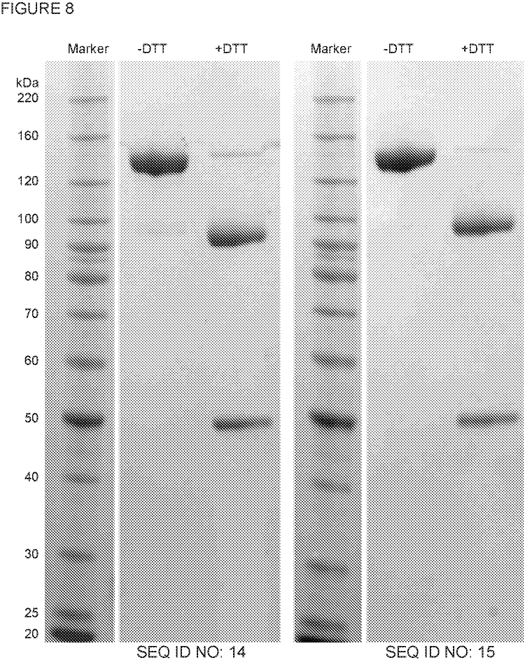

FIG. 9 shows that chimera 3B and 3C displayed greater potency than rBoNT/A1 in cleaving SNAP-25 in induced human pluripotent stem cells but the former significantly more so. This can be explained by the double mutation which increases the affinity of chimera 3B for the human synaptotagmin II protein receptor present in these cells (FIG. 9, Table 7).

TABLE 7

pEC₅₀ values.

| | pEC₅₀ ±SEM |
|---|---|
| rBoNT/A1 | 10.21 ±0.05 |
| Chimera 3B | 12.38 ±0.06 |
| Chimera 3C | 10.72 ±0.08 |

### Digit Abduction Scoring (DAS) Assay — Safety Ratio

The method to measure the activity of BoNTs in the DAS assay is based on the startled response toe spreading reflex of mice, when suspended briefly by the tail. This reflex is scored as Digit Abduction Score (DAS) and is inhibited after administration of BoNT into the gastrocnemius-soleus muscles of the hind paw. Mice are suspended briefly by the tail to elicit a characteristic startled response in which the animal extends its hind limb and abducts its hind digits. (Aoki et al. 1999, Eur. J. Neurol.; 6 (suppl. 4) S3-S10).

On the day of injection, mice were anaesthetized in an induction chamber receiving isoflurane 3% in oxygen. Each mouse received an intramuscular injection of BoNT or vehicle (phosphate buffer containing 0.2 % gelatine) in the gastrocnemius-soleus muscles of the right hind paw.

Following neurotoxin injection, the varying degrees of digit abduction were scored on a scale from zero to four, where 0= normal and 4= maximal reduction in digit abduction and leg extension. ED50 was determined by nonlinear adjustment analysis using average of maximal effect at each dose. The mathematical model used was the 4 parameters logistic model.

DAS was performed every 2 hours during the first day after dosing; thereafter it was performed 3 times a day for 4 days for all doses. Animals of the groups injected with vehicle and the lowest dose that induced during the first four days of injection a DAS of 4 were thereafter monitored until complete recovery of the muscle weakness to a DAS of 0 (no observed muscle weakness).

For calculation of the safety ratio all animals were weighed the day before toxin injection (D0) and thereafter once daily throughout the duration of the study. The average body weight, its standard deviation, and the standard error mean were calculated daily for each dose-group. To obtain the safety ratio for a BoNT (-10%ΔBW/ED₅₀), the dose at which at any time during the study the average weight of a dose-group was lower than 10% of the average weight at D0 of that same dose-group was divided by the ED₅₀ for the BoNT studied. The lethal dose was defined as the dose at which one or more of the animals within that dose-group died.

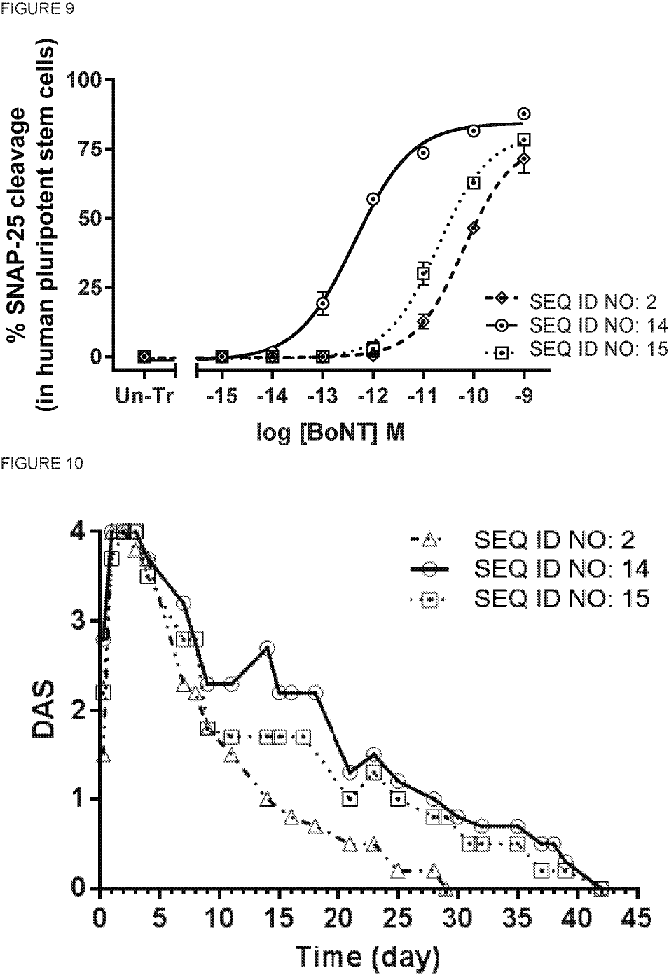

FIG. 10 shows the duration of muscle weakening over time in the mouse digit abduction scoring assay for unmodified BoNT/A, chimera 3B and chimera 3C (SEQ ID NO: 2, 14 and 15), showing that the chimera has longer duration of action.

Table 8 below provides the ED₅₀ and DAS 4 doses determined for rBoNT/A1 and chimeras 3B and 3C in the mouse DAS assay. The table also provide the total duration of action for the DAS 4 dose until complete recovery of the muscle weakness to a DAS of 0 (no observed muscle weakness). In addition, the table shows the mouse lethal dose and the safety ratio (-10%ΔBW/ED₅₀), as defined in the text above. In comparison to rBoNT/A1, chimeras 3B and 3C have longer duration of action, a better safety ratio, and a higher lethal dose. Studies shown in FIG. 10 and Table 8 were performed in mice obtained from Janvier laboratories.

TABLE 8

DAS and Safety Ratios of the BoNT/AB chimeras.

| | ED₅₀ (DAS 2) Dose (pg/mouse) | DAS 4 dose (pg/mouse) | Total duration of action (day) with lowest DAS 4 dose | Mouse lethal dose (pg) | Safety ratio (-10%ΔBW/ED₅₀) |
|---|---|---|---|---|---|
| rBoNT/A1 | 0.9 | 2.3 | 29 | 18 | 4.5 |
| Chimera 3B | 8.0 | 89 | 42 | 200 | 14.1 |
| Chimera 3C | 5.0 | 26 | 42 | 8.9 | 7.4 |

### Example 6

### Pre-Clinical Testing of Modified BoNT/A (SEQ ID NO: 4)

The modified BoNT/A “Cat-A” (SEQ ID NO: 4) was subjected to additional pre-clinical testing.

### Materials & Methods

### Rat Digit Abduction Score (DAS) Assay

To assess the effects of modified BoNT/A (SEQ ID NO: 4) on in vivo muscular activity, dose-response studies were conducted using the rat DAS assay. The rat DAS assay is based on the toe spreading reflex, a characteristic startle response, when the animal is briefly grasped.

Following a single neurotoxin injection into the left peroneus muscle complex, the muscular weakness results in a reduction in digit abduction. The varying degrees of digit abduction are scored on a 5-point scale: 0=normal to 4=maximal reduction in digit abduction and leg extension (Broide RS, Rubino J, Nicholson GS, et al. The rat Digit Abduction Score (DAS) assay: A physiological model for assessing botulinum neurotoxin-induced skeletal muscle paralysis. Toxicon 2013;71:18-24). DAS values were measured for the first five consecutive days after toxin injection and after this at intervals of two to three days until complete disappearance of the effect of modified BoNT/A (SEQ ID NO: 4) on the toe spreading reflex for lower doses and until recovery to DAS2 for doses resulting in DAS4. Transient BoNT-induced dose-dependent effects on body weight gain are considered evidence of a generalised toxin effect (Torii Y, Goto Y, Nakahira S, et al. Comparison of Systemic Toxicity between Botulinum Toxin Subtypes A1 and A2 in Mice and Rats. Basic Clin. Pharmacol. Toxicol. 2015;116:524-528.). At each evaluation time point rats were consequently weighed and side effects were noted. Dosing solutions of BoNT were masked (assigned random letters) before injection and until the end of the study. Potency was determined as the dose required to induce 50% of the effect (ED₅₀: dose leading to a DAS value of 2). To determine ED₅₀ and the 95% confidence intervals (Cls), doses ranging between 2.5 and 750 pg/kg were tested. Higher doses of 1, 1.5, 2, 2.4, 3, 4 and 5 ng/kg were also administered to assess possible side effects.

To evaluate the duration of action of modified BoNT/A (SEQ ID NO: 4) and compare it to the duration of action of unmodified BoNT/A (SEQ ID NO: 2), the median time necessary to return to a DAS2 reading of 2 was evaluated for the highest tolerated dose (no impact on body weight evolution compared to untreated rats) for both toxins in two independent, direct head-to-head studies.

### Rat Single Dose Studies

Rats received a single intramuscular (i.m.) injection of modified BoNT/A (SEQ ID NO: 4) at doses of 0, 0.1, 1 and 3 ng/kg administered into the right gastrocnemius muscle. Control animals received SEQ ID NO: 4 diluent in the right gastrocnemius. Animals were euthanised 7 days after treatment (ten males and ten females per group) or after a 13 or 26-week observation period (five males and five females per dose). Irwin test observations, for assessment of central nervous system function, were performed pretest (Day -1), on Day 8 and during Weeks 13 and 27. Other clinical (adverse) signs assessed for were limping, small toxin injected muscle size, and soft distended abdomen.

### Monkey Studies

Monkeys received single i.m. doses of 0, 0.1, 0.25 and 0.75 ng/kg modified BoNT/A (SEQ ID NO: 4) administered into the right gastrocnemius muscle. Animals were euthanised 7 days after treatment (three males and three females per group) or after a 13 or 26-week observation period (two males and two females per dose). Cardiovascular examinations, including haemodynamic, electrocardiogram and respiratory parameters, were performed by external telemetry pretest, on Days 8 and 15.

### Preliminary Enhanced EFD in Pregnant Rat

The objective of the study was to provide initial information on the effects of modified BoNT/A (SEQ ID NO: 4) on embryonic and foetal development of the rat when administered by the i.m. route throughout the period of organogenesis. Modified BoNT/A (SEQ ID NO: 4) was administered by daily i.m. injection (gastrocnemius) at dose levels of 0.02, 0.05 and 0.1 ng/kg/day to groups of nine mated female Sprague-Dawley rats from days 6 (G6) to 17 (G17) of gestation, inclusive. Clinical condition, body weight and food consumption were monitored throughout the study. The females were submitted to a caesarean examination on G21 and litter parameters were recorded. At necropsy, the females were examined macroscopically, the gravid uteri were weighed and for those who presented a small injected gastrocnemius muscle, this muscle and the contralateral muscle were weighed. All foetuses were weighed. The foetuses were then examined for external and visceral abnormalities and sexed. The heads of approximately half of the foetuses were fixed for internal examination by serial sectioning. The eviscerated carcasses of all fetuses were processed for skeletal examination.

### Preliminary Extended EFD in Pregnant Rabbit

The objective of the study was to provide initial information on the effects of modified BoNT/A (SEQ ID NO: 4) on embryonic and foetal development of the rabbit when administered by the i.m. route throughout the period of organogenesis. Modified BoNT/A (SEQ ID NO: 4) was administered by daily i.m. injection (gastrocnemius) at dose levels of 0.002, 0.005 and 0.01 ng/kg/day to groups of nine mated female New Zealand White rabbits from days 6 (G6) to 19 (G19) of gestation, inclusive. Clinical condition, body weight and food consumption were monitored throughout the study. The females were submitted to a caesarean examination on G29 and litter parameters were recorded. At necropsy, the females were examined macroscopically, the gravid uteri were weighed and for those who presented a small injected gastrocnemius muscle, this muscle and the contralateral muscle were weighed. All foetuses were weighed. The foetuses were then examined for external and visceral abnormalities and sexed. The heads of approximately half of the foetuses were fixed for internal examination by serial sectioning.

### Results

By carrying out the studies as indicated above, the following pharmacological data (indicated in Table 9 below) were obtained for a number of different species administered the modified BoNT/A.

TABLE 9

Pre-clinical results.

| | | |
|---|---|---|
| Animal | Study Type | Results |
| Mouse | LD50 IP | 0.422 ng/kg |
| Rat | DAS | ED50 0.013 ng/kg DAS4 0.125 pg/kg |
| | CMAP Single Dose Distant Spread | 0.002 ng/kg: No spread 0.3 ng/kg: -25% 0.8 ng/kg: -56% |
| | Single Dose | Estimated NOAEL 1.5 ng/kg Estimated Lethal 3 ng/kg |

Additionally, modified BoNT/A (SEQ ID NO: 4) was tested in a rat DAS assay to determine the duration of action when compared to Dysport®. Results are presented in Table 10 below:

TABLE 10

Duration of action.

| | Dysport® 3 U/rat 15 U/kg | Modified BoNT/A 150 pg/rat 0.750 ng/kg |
|---|---|---|
| Duration of Action (median days) | 21.9 | 46.4 |

These data show that the modified BoNT/A has a duration of action that is more than double that of Dysport®.

### Example 7

### Calculation of a Unit Dose of Modified BoNT/A (SEQ ID NO: 4) for Treating Facial Lines

In view of the pre-clinical pharmacology data obtained in Example 6 above, a suitable unit dose range (UD) for administration of modified BoNT/A in humans has been calculated. The studies show that modified BoNT/A provides a longer duration of action than unmodified BoNT/A while at the same time exhibiting an improved safety profile. This improved safety profile may be expressed by the high Safety Ratio described herein for the modified BoNT/A.

As modified BoNT/A shares the same mechanism of action as Dysport® (albeit with an increased therapeutic index (i.e. an increased Safety Ratio) due to its modified properties), the lowest dose of modified BoNT/A for treating subjects with facial lines has been positioned for context relative to the labelled doses of Dysport® in the same muscle groups:
In the Digit Abduction Score rat model, the ED₅₀ of modified BoNT/A generated with the batch intended for clinical use is 13 pg/kg, and is more than 100-fold lower than the estimated no-observed-adverse-effect-level (NOAEL) of 1500 pg/kg in the same animal species. Following application of the corresponding allometric factor, this ED50 can be translated into a human equivalent dose (HED) of 130 pg. Based on DAS rat findings, 100 pg of modified BoNT/AB would eastimate to a dose of approximately 4 U of marketed Azzalure/Dysport. If the same approach is applied for calculation of a starting dose using the DAS rat ED50 established for Azzalure/Dysport (i.e., an ED50 of 0.5 U/kg), this would equate to a total starting dose of 5 U which is ⅒ of the total registered dose of 50 U for the temporary improvement of moderate to severe GL.
The intraperitoneal mouse LD50 was established at 0.422 ng/kg (8.44 pg/animal). Under these conditions, a dose of 0.84 ng of modified BoNT/A corresponds to a dose of 100 U Dysport®.

The calculated lowest starting total dose is thus 100 pg. To provide some context and using the intraperitoneal mouse LD₅₀ data above, 100 pg modified BoNT/A equates to approximately 12 U Dysport® and would thus be active when administered intramuscularly for treatment of facial lines.

The upper limit of the unit dose is thus calculated to be 300 pg (0.3 ng), with a total dose administered to the subject of 4800 pg as this remains below the rat NOAEL translated in human dose.

Thus, a suitable unit dose for treatment of facial lines using modified BoNT/A has been calculated at 20-300 pg. Based on the pre-clinical data obtained, this is 2-35 Units of modified BoNT/A based on the calculated median lethal intraperitoneal dose (LD₅₀) in mice as determined using the mouse intraperitoneal Lethal Dose Assay.

Advantageously, modified BoNT/A can be injected to a greater number of muscles in the treatment of facial lines before reaching the maximum dose. This is a significant and advantageous finding leading to improved treatment of upper facial lines while providing clinicians with a greater range of treatment options.

### Example 8

### Dosage Regimen for Treating Glabellar Lines

Modified BoNT/A is provided as a lyophilised powder in 2 mL clear glass vials containing 15 ng of modified BoNT/A per vial. The lyophilised powder is reconstituted with a mixture of sterile sodium chloride 0.9% v/w preservative free solution and diluent (formulation buffer containing only the excipients of modified BoNT/A). After reconstitution, the solution is further diluted as necessary.

Moderate to severe glabellar lines are treated according to the injection regimen shown in FIG. 11.

The unit dose is 20-300 pg (2-35 Units).

Intramuscular injections are administered at up to five sites according to the unit dose. A maximum total dosage administered is 1500 pg (177 Units).

### Example 9

### Dosage Regimen for Treating Glabellar Lines and Forehead Lines

Modified BoNT/A is provided as a lyophilised powder in 2 mL clear glass vials containing 15 ng of modified BoNT/A per vial. The lyophilised powder is reconstituted with a mixture of sterile sodium chloride 0.9% v/w preservative free solution and diluent (formulation buffer containing only the excipients of modified BoNT/A). After reconstitution, the solution is further diluted as necessary.

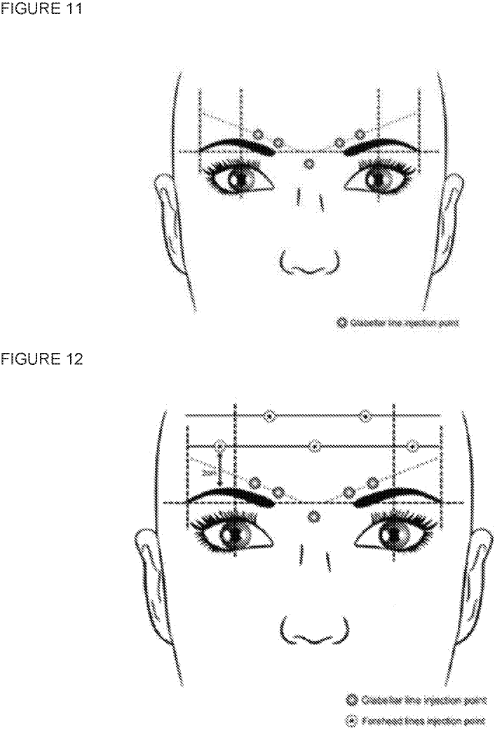

Moderate to severe glabellar and forehead lines are treated according to the injection regimen shown in FIG. 12.

The unit dose is 20-300 pg (2-35 Units).

Intramuscular injections are administered at up to ten sites according to the unit dose. A maximum total dosage administered is 3000 pg (355 Units).

### Example 10

### Dosage Regimen for Treating Lateral Canthal Lines

Modified BoNT/A is provided as a lyophilised powder in 2 mL clear glass vials containing 15 ng of modified BoNT/A per vial. The lyophilised powder is reconstituted with a mixture of sterile sodium chloride 0.9% v/w preservative free solution and diluent (formulation buffer containing only the excipients of modified BoNT/A). After reconstitution, the solution is further diluted as necessary.

Moderate to severe lateral canthal lines are treated according to the injection regimen shown in FIG. 13.

The unit dose is 20-300 pg (2-35 Units).

Intramuscular injections are administered at up to six sites according to the unit dose. A maximum total dosage administered is 1800 pg (213 Units).

### Example 11

### Dosage Regimen for Treating Glabellar, Forehead and Lateral Canthal Lines

Modified BoNT/A is provided as a lyophilised powder in 2 mL clear glass vials containing 15 ng of modified BoNT/A per vial. The lyophilised powder is reconstituted with a mixture of sterile sodium chloride 0.9% v/w preservative free solution and diluent (formulation buffer containing only the excipients of modified BoNT/A). After reconstitution, the solution is further diluted as necessary.

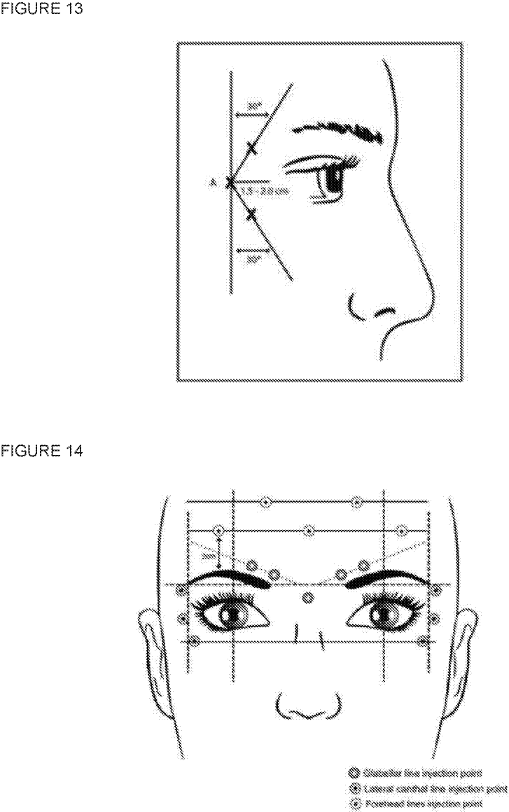

Moderate to severe glabellar, forehead and lateral canthal lines are treated according to the injection regimen shown in FIG. 14.

The unit dose is 20-300 pg (2-35 Units).

Intramuscular injections are administered at up to sixteen sites according to the unit dose. A maximum total dosage administered is 4800 pg (569 Units).

### Example 12

### Further Characterisation of a Modified BoNT/A (SEQ ID NO: 14)

BoNT/AB chimera SEQ ID NO: 14 was tested in a mouse LD₅₀ assay yielding a result of 1.202 ng/kg. 1 Unit of SEQ ID NO: 14 therefore corresponds to 24.04 pg in this assay.

Additionally, said BoNT/AB chimera was tested in a rat DAS assay to determine the duration of action (as per Example 6) when compared to Dysport®. Results are presented in Table 13 below:

TABLE 13

Duration of action.

| | Dysport® 3 U/rat 15 U/kg | BoNT/AB 300 pg/rat 1.5 ng/kg |
|---|---|---|
| Duration of Action (median days) | 21.9 | 47.7 |

In conclusion, the duration of action of BoNT/AB was much higher than Dysport® and similar to that of SEQ ID NO: 4. Thus, it is expected that the unit doses and dosage regimen for SEQ ID NO: 4 could similarly be applied to BoNT/AB to provide an improved treatment of facial lines.

### Example 13

### Calculation of a Unit Dose of Modified BoNT/A (SEQ ID NO: 14) for Upper Facial Lines

In view of pre-clinical pharmacology data, a suitable unit dose range (UD) for administration of modified BoNT/A in humans has been calculated.

A DAS ED₅₀ of 13 pg/kg was calculated for SEQ ID NO: 14. ED₅₀ is considered as a minimal pharmacologically active dose, which is approximately 300-fold lower than the no observed adverse effect level (NOAEL) of 4 ng/kg in the same animal species. An ED₅₀ of 13 pg/kg of SEQ ID NO: 14 in rats corresponds to a 0.8 ng dose for a human of 60 kg body weight.

Thus, the lower limit of a unit dose of 20 pg was selected. An upper limit of the unit dose of 1500 pg was selected, which is lower than the NOAEL of 4 ng/kg from both nonclinical safety species (rat and monkey) converted into human dose for 60 kg body weight.

In view of the improved safety profile the maximum total dose for the treatment of upper facial lines was set at 24,000 pg, which is derived from the NOAEL of 4 ng/kg from both nonclinical safety species (rat and monkey) converted into human dose for 60 kg body weight.

### Example 14

### Dosage Regimen for Treating Glabellar Lines Using a Modified BoNT/A (SEQ ID NO: 14)

Modified BoNT/A is provided as a lyophilised powder in a vial containing 36 ng of modified BoNT/A per vial. The lyophilised powder is reconstituted.

Moderate to severe glabellar lines are treated according to the injection regimen shown in FIG. 11.

The unit dose is 20-1500 pg (0.8-62 Units).

Intramuscular injections are administered at up to five sites according to the unit dose. A maximum total dosage administered is 7500 pg (312 Units).

### Example 15

### Dosage Regimen for Treating Glabellar Lines and Forehead Lines Using a modified BoNT/A (SEQ ID NO: 14)

Modified BoNT/A is provided as a lyophilised powder in a vial containing 36 ng of modified BoNT/A per vial. The lyophilised powder is reconstituted.

Moderate to severe glabellar and forehead lines are treated according to the injection regimen shown in FIG. 12.

The unit dose is 20-1500 pg (0.8-62 Units).

Intramuscular injections are administered at up to ten sites according to the unit dose. A maximum total dosage administered is 15,000 pg (624 Units).

### Example 16

### Dosage Regimen for Treating Lateral Canthal Lines Using a Modified BoNT/A (SEQ ID NO: 14)

Modified BoNT/A is provided as a lyophilised powder in a vial containing 36 ng of modified BoNT/A per vial. The lyophilised powder is reconstituted.

Moderate to severe lateral canthal lines are treated according to the injection regimen shown in FIG. 13.

The unit dose is 20-1500 pg (0.8-62 Units).

Intramuscular injections are administered at up to six sites according to the unit dose. A maximum total dosage administered is 9000 pg (374 Units).

### Example 17

### Dosage Regimen for Treating Glabellar, Forehead and Lateral Canthal Lines Using A Modified BoNT/A (SEQ ID NO: 14)

Modified BoNT/A is provided as a lyophilised powder in a vial containing 36 ng of modified BoNT/A per vial. The lyophilised powder is reconstituted.

Moderate to severe glabellar, forehead and lateral canthal lines are treated according to the injection regimen shown in FIG. 14.

The unit dose is 20-1500 pg (0.8-62 Units).

Intramuscular injections are administered at up to sixteen sites according to the unit dose. A maximum total dosage administered is 24,000 pg (998 Units).

All publications mentioned in the above specification are herein incorporated by reference. Various modifications and variations of the described methods and system of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in biochemistry and biotechnology or related fields are intended to be within the scope of the following claims.

1. A modified BoNT/A for use in treating facial lines, wherein the modified BoNT/A is administered by intramuscular injection at a plurality of sites of the face of the subject,
wherein the modified BoNT/A is administered by way of a unit dose of 1 Units to 41 Units of modified BoNT/A per site and wherein 1 Unit is an amount of the modified BoNT/A that corresponds to the calculated median lethal dose (LD₅₀) in mice,
wherein the plurality of sites are selected from:
up to two sites of a corrugator muscle and one site of a procerus muscle for treating glabellar lines,
up to five sites of a frontalis muscle for treating forehead lines; and
up to three sites at the external part of an orbicularis oculi muscle for treating lateral canthal lines,
wherein the total dose of modified BoNT/A administered during the treatment is up to 574 Units, and
wherein the modified BoNT/A comprises a modification at one or more amino acid residue(s) selected from: ASN 886, ASN 905, GLN 915, ASN 918, GLU 920, ASN 930, ASN 954, SER 955, GLN 991, GLU 992, GLN 995, ASN 1006, ASN 1025, ASN 1026, ASN 1032, ASN 1043, ASN 1046, ASN 1052, ASP 1058, HIS 1064, ASN 1080, GLU 1081, GLU 1083, ASP 1086, ASN 1188, ASP 1213, GLY 1215, ASN 1216, GLN 1229, ASN 1242, ASN 1243, SER 1274, and THR 1277, wherein the modification is selected from:
i. substitution of an acidic surface exposed amino acid residue with a basic amino acid residue;
ii. substitution of an acidic surface exposed amino acid residue with an uncharged amino acid residue;
iii. substitution of an uncharged surface exposed amino acid residue with a basic amino acid residue;
iv. insertion of a basic amino acid residue; and
v. deletion of an acidic surface exposed amino acid residue.

2. A modified BoNT/A for use in treating facial lines, wherein the modified BoNT/A is administered by intramuscular injection at a plurality of sites of the face of the subject,
wherein the modified BoNT/A is administered by way of a unit dose of 8.4 pg to 344.4 pg of modified BoNT/A per site,
wherein the plurality of sites are selected from:
up to two sites of a corrugator muscle and one site of a procerus muscle for treating glabellar lines,
up to five sites of a frontalis muscle for treating forehead lines; and
up to three sites at the external part of an orbicularis oculi muscle for treating lateral canthal lines,
wherein the total dose of modified BoNT/A administered during the treatment is up to 4821.6 pg, and
wherein the modified BoNT/A comprises a modification at one or more amino acid residue(s) selected from: ASN 886, ASN 905, GLN 915, ASN 918, GLU 920, ASN 930, ASN 954, SER 955, GLN 991, GLU 992, GLN 995, ASN 1006, ASN 1025, ASN 1026, ASN 1032, ASN 1043, ASN 1046, ASN 1052, ASP 1058, HIS 1064, ASN 1080, GLU 1081, GLU 1083, ASP 1086, ASN 1188, ASP 1213, GLY 1215, ASN 1216, GLN 1229, ASN 1242, ASN 1243, SER 1274, and THR 1277, wherein the modification is selected from:
i. substitution of an acidic surface exposed amino acid residue with a basic amino acid residue;
ii. substitution of an acidic surface exposed amino acid residue with an uncharged amino acid residue;
iii. substitution of an uncharged surface exposed amino acid residue with a basic amino acid residue;
iv. insertion of a basic amino acid residue; and
v. deletion of an acidic surface exposed amino acid residue.

3. The modified BoNT/A for use according to claim 1, wherein 5-10 pg (preferably 8.4 pg) of the modified BoNT/A corresponds to the calculated median lethal dose (LD₅₀) in mice.

4. The modified BoNT/A for use according to any one of the preceding claims, wherein the unit dose of modified BoNT/A is selected from: 2 Units to 35 Units, 6 to 35 Units, 12 Units to 35 Units, 12 Units to 24 Units, most preferably 12 Units to 18 Units.

5. The modified BoNT/A for use according to claim 4, wherein the total dose of modified BoNT/A administered during the treatment is selected from: 574 Units, 560 Units, 384 Units, most preferably 288 Units;
preferably wherein said total dose is based on administration of 16 unit doses across 16 injection sites.

6. The modified BoNT/A for use according to any one of the preceding claims, wherein the unit dose of modified BoNT/A is selected from: 18 pg to 350 pg, 20 pg to 300 pg, most preferably 100 pg to 150 pg.

7. The modified BoNT/A for use according to any one of the preceding claims, wherein the total dose of modified BoNT/A administered during the treatment is selected from up to: 4850 pg, 4800 pg, most preferably 2400 pg.

8. A modified BoNT/A for use in treating facial lines, wherein the modified BoNT/A is administered by intramuscular injection at a plurality of sites of the face of the subject,
wherein the modified BoNT/A is administered by way of a unit dose of 0.5 Units to 73 Units of modified BoNT/A per site and wherein 1 Unit is an amount of the modified BoNT/A that corresponds to the calculated median lethal dose (LD₅₀) in mice,
wherein the plurality of sites are selected from:
up to two sites of a corrugator muscle and one site of a procerus muscle for treating glabellar lines,
up to five sites of a frontalis muscle for treating forehead lines; and
up to three sites at the external part of an orbicularis oculi muscle for treating lateral canthal lines,
wherein the total dose of modified BoNT/A administered during the treatment is up to 1019 Units, and
wherein the modified BoNT/A comprises a BoNT/A light-chain and translocation domain, and a BoNT/B receptor binding domain (H_{c} domain).

9. A modified BoNT/A for use in treating facial lines, wherein the modified BoNT/A is administered by intramuscular injection at a plurality of sites of the face of the subject,
wherein the modified BoNT/A is administered by way of a unit dose of 12 pg to 1754 pg of modified BoNT/A per site,
wherein the plurality of sites are selected from:
up to two sites of a corrugator muscle and one site of a procerus muscle for treating glabellar lines,
up to five sites of a frontalis muscle for treating forehead lines; and
up to three sites at the external part of an orbicularis oculi muscle for treating lateral canthal lines,
wherein the total dose of modified BoNT/A administered during the treatment is up to 24,500 pg, and
wherein the modified BoNT/A comprises a BoNT/A light-chain and translocation domain, and a BoNT/B receptor binding domain (H_{c} domain).

10. The modified BoNT/A for use according to claim 8, wherein the modified BoNT/A comprises a BoNT/A light-chain and translocation domain, and a BoNT/B H_{c} domain, and wherein 20-30 pg (preferably 24.04 pg) of the modified BoNT/A corresponds to the calculated median lethal dose (LD₅₀) in mice.

11. The modified BoNT/A for use according to any one of claims 8-10, wherein the unit dose of modified BoNT/A is selected from: 0.5 Units to 62 Units, 3 Units to 62 Units, 10 to 62 Units, 10 Units to 42 Units, most preferably 10 Units to 21 Units.

12. The modified BoNT/A for use according to claim 11, wherein the total dose of modified BoNT/A administered during the treatment is selected from: 1019 Units, 998 Units, 666 Units, most preferably 333 Units;
preferably wherein said total dose is based on administration of 16 unit doses across 16 injection sites.

13. The modified BoNT/A for use according to any one of claims 8-12, wherein the unit dose of modified BoNT/A is selected from: 12 pg to 1750 pg, 20 pg to 1500 pg, most preferably 250 pg to 500 pg.

14. The modified BoNT/A for use according to any one of claims 8-13, wherein the total dose of modified BoNT/A administered during the treatment is selected from up to: 24,500 pg, 24,000 pg, most preferably 8000 pg.

15. The modified BoNT/A for use according to any one of the preceding claims, wherein the modified BoNT/A has a Safety Ratio of greater than 7, wherein the Safety Ratio is calculated as: dose of toxin required for -10% bodyweight change measured as pg/mouse divided by DAS ED₅₀ measured as pg/mouse, wherein ED₅₀ = dose required to produce a DAS score of 2.

16. The modified BoNT/A for use according to any one of the preceding claims, wherein the modified BoNT/A is for the use in treating glabellar lines, preferably wherein said modified BoNT/A is administered at two sites of a corrugator muscle and one site of a procerus muscle, more preferably wherein said modified BoNT/A is administered at a total of five sites.

17. The modified BoNT/A for use according to claims 1-15, wherein the modified BoNT/A is for the use in treating forehead lines, preferably wherein said modified BoNT/A is administered at five sites of a frontalis muscle.

18. The modified BoNT/A for use according to claims 1-15, wherein the modified BoNT/A is for the use in treating lateral canthal lines, preferably wherein said modified BoNT/A is administered at three sites at the external part of a orbicularis oculi muscle for treating lateral canthal lines, more preferably wherein said modified BoNT/A is administered at a total of six sites.

19. The modified BoNT/A for use according to claims 16-18, wherein said modified BoNT/A is administered to 8, 10, 12, 14 or 16 sites.

20. The modified BoNT/A for use according to claims 16-18, wherein said modified BoNT/A is administered to 7, 9, 11, 13 or 15 sites.

21. The modified BoNT/A for use according to claims 1-7 or 15-20, wherein said modification comprises (preferably consists of) a modification at one or more amino acid residue(s) selected from: ASN 886, ASN 930, ASN 954, SER 955, GLN 991, ASN 1025, ASN 1026, ASN 1052, ASN 1188, ASP 1213, GLY 1215, ASN 1216, GLN 1229, ASN 1242, ASN 1243, SER 1274 or THR 1277, and wherein the modified BoNT/A is encoded by a nucleic acid sequence having at least 70% sequence identity to a nucleic acid sequence selected from SEQ ID NOs: 3, 5, 7, and 9, and/or comprises a polypeptide sequence having at least 70% sequence identity to a polypeptide sequence selected from SEQ ID NOs: 4, 6, 8, and 10.

22. The modified BoNT/A for use according to claims 1-7 or 15-21, wherein said modification comprises (preferably consists of) a modification at one or more amino acid residue(s) selected from: ASN 886, ASN 930, SER 955, GLN 991, ASN 1026, ASN 1052, and GLN 1229, and wherein the modified BoNT/A is encoded by a nucleic acid sequence having at least 70% sequence identity to SEQ ID NO: 3, and/or comprises a polypeptide sequence having at least 70% sequence identity to an amino acid sequence selected from SEQ ID NO: 4.

23. The modified BoNT/A for use according to claims 1-7 or 15-22, wherein the modification is a substitution, preferably a substitution with lysine or arginine.

24. The modified BoNT/A for use according to any one of claims 8-20, wherein the modified BoNT/A comprises a polypeptide sequence having at least 70% sequence identity to SEQ ID NO: 14.

25. A unit dosage form of modified botulinum neurotoxin A (BoNT/A), the unit dosage form comprising:
a. 1 Units to 41 Units of modified BoNT/A, wherein 1 Unit is an amount of the modified BoNT/A that corresponds to the calculated median lethal dose (LD₅₀) in mice; or
b. 8.4 pg to 350 pg of modified BoNT/A; and
c. optionally a pharmaceutically acceptable carrier, excipient, adjuvant, and/or salt,
d. wherein the modified BoNT/A comprises a modification at one or more amino acid residue(s) selected from: ASN 886, ASN 905, GLN 915, ASN 918, GLU 920, ASN 930, ASN 954, SER 955, GLN 991, GLU 992, GLN 995, ASN 1006, ASN 1025, ASN 1026, ASN 1032, ASN 1043, ASN 1046, ASN 1052, ASP 1058, HIS 1064, ASN 1080, GLU 1081, GLU 1083, ASP 1086, ASN 1188, ASP 1213, GLY 1215, ASN 1216, GLN 1229, ASN 1242, ASN 1243, SER 1274, and THR 1277, wherein the modification is selected from:
i. substitution of an acidic surface exposed amino acid residue with a basic amino acid residue;
ii. substitution of an acidic surface exposed amino acid residue with an uncharged amino acid residue;
iii. substitution of an uncharged surface exposed amino acid residue with a basic amino acid residue;
iv. insertion of a basic amino acid residue; and
v. deletion of an acidic surface exposed amino acid residue.

26. The unit dosage form according to claim 25, comprising:
a. 1 Units to 35 Units of modified BoNT/A; or
b. 10 pg to 300 pg of modified BoNT/A.

27. A unit dosage form of modified botulinum neurotoxin A (BoNT/A), the unit dosage form comprising:
a. 0.5 Units to 73 Units of modified BoNT/A, wherein 1 Unit is an amount of the modified BoNT/A that corresponds to the calculated median lethal dose (LD₅₀) in mice; or
b. 12 pg to 1754 pg of modified BoNT/A; and
c. optionally a pharmaceutically acceptable carrier, excipient, adjuvant, and/or salt,
d. wherein the modified BoNT/A comprises a BoNT/A light-chain and translocation domain, and a BoNT/B receptor binding domain (H_{c} domain).

28. The unit dosage form according to claim 27, comprising:
a. 0.8 Units to 62 Units of modified BoNT/A; or
b. 20 pg to 1500 pg of modified BoNT/A.

29. A kit comprising:
a. the unit dosage form according to any one of claims 25-28; and
b. instructions for use of the same in treating facial lines; and
c. optionally a diluent.
